(19) United States
(12) Patent Application Publication
(10) Pub. No.: US 2015/0211012 A1
(43) Pub. Date: Jul. 30, 2015

(54) MYCOBACTERIUM COMPRISING EXPRESSION VECTOR WITH TWO AUXOTROPHIC SELECTION MARKERS AND ITS USE AS VACCINE

(71) Applicants: LABORATORIOS DEL DR. ESTEVE S.A., Barcelona (ES); FUNDACIÓ PRIVADA INSTITUT DE RECERCA DE LA SIDA - CAIXA, Badalona (ES)

(72) Inventors: Joan Joseph, Barcelona (ES); Narcis Saubi Roca, Girona (ES)

(21) Appl. No.: 14/424,545

(22) PCT Filed: Jul. 2, 2013
(86) PCT No.: PCT/EP2013/063978

§ 371 Date: Feb. 27, 2015

(30) Foreign Application Priority Data
Aug. 31, 2012 (EP) 12382336.1

(51) Int. Cl. C12N 15/70; A61K 39/21; C12N 15/74

(52) U.S. Cl. CPC C12N 15/70; C12N 15/74; A61K 39/21; A61K 2039/523

(57) ABSTRACT

The invention relates to polynucleotides and recombinant cell strains comprising the polynucleotides and the uses thereof for the delivery of the polypeptides encoded by the polynucleotides to a subject in need thereof. In particular, the invention refers to polynucleotides comprising a polypeptide of interest, auxotrophy-complementing genes and the use thereof in a mycobacterial double auxotrophic host cell to achieve the stable expression of the polypeptide of interest by using an antibiotic-free plasmid selection system.

### FIELD OF THE INVENTION

The invention relates to the field of immunology and, more in particular, to methods and compositions for inducing an immune response against an antigen of interest by administering said antigen through a recombinant mycobacterial double auxotrophic strain. The invention relates also to methods for expressing a gene of interest by utilizing a mycobacterial double auxotrophic strain.

### BACKGROUND OF THE INVENTION

Vaccines are the most cost-effective intervention to prevent disease. Mycobacterium bovis BCG offers great potential for innovative approaches for the development of polyvalent vaccines. Novel vaccine candidates, such as HIV-1 related immunogens, could use BCG as a live bacterial vaccine vehicle to elicit more effective cellular and humoral responses.

There is strong evidence supporting a role of cytotoxic T lymphocytes (CTLs) in the containment of HIV replication and several vaccine approaches are being pursued to elicit anti-HIV CTL responses. CTL induction against HIV-1 and simian immunodeficiency virus (SIV) gag or env antigens has been described following the immunization of mice or rhesus monkeys with recombinant BCG (rBCG) expressing these antigens. See Ohara N, et al., Vaccine 2001; 19:4089-4098). More recently, recombinant Mycobacterium bovis Bacillus Calmette-Guerin (BCG) expressing HIVA immunogen has been generated and shown to be stable and to induce durable, high-quality HIV-1-specific CD4+ and CD8+ T-cell responses in BALB/c mice. Furthermore, when the recombinant BCG vaccine was used in a prime-boost regimen with heterologous vectors expressing the same HIV immunogen, the HIV-1-specific responses provided protection against surrogate virus challenge, and the recombinant BCG vaccine alone protected against aerosol challenge with M. tuberculosis. See Im E, et al., J. Virol. 2007; 81:9408-9418.

Critical issues to be considered in developing rBCG technology include: i) antigen localization, ii) codon optimization and iii) in vivo plasmid DNA stability and genetic rearrangements. In vivo genetic stability and persistence are of special importance for the use of live bacterial vaccines; a mutant BCG which is rapidly eliminated is unlikely to be an effective vaccine. It is known that BCG undergoes significant genetic rearrangements. Recent evidence suggests that major recombination events resulting in the duplication of large segments of its chromosome have occurred and are still occurring. Thus, if BCG is to be used as a live bacterial delivery system for generating novel vaccine candidates and as an immunotherapeutic agent, it is essential that a more genetically stable strain be developed.

The possibility of inserting foreign genes into the chromosome at precise positions to ensure the persistence of the heterologous genetic information in the recombinant vaccine strains would represent a crucial step in the development of Mycobacterium bovis BCG as a live bacterial delivery system for expression of heterologous antigens. Accordingly, there is a need for better vaccine delivery systems based on Mycobacterium bovis BCG which could overcome the problems of the vaccines described in the art.

### SUMMARY OF THE INVENTION

The present invention provides a polynucleotide that can be expressed in a recombinant host cell and used for the delivery of a polypeptide of interest to a subject in need thereof. Typically, the propagation of the polynucleotide is carried out in a conventional host cell such as E. coli, whereas the expression for therapeutic delivery is carried out in mycobacterial cells. Therefore, it is convenient that the polynucleotide of the invention further comprises elements which allow the selection of those cells carrying the polynucleotide from those which do not contain said polynucleotide. Historically, antibiotic resistance genes have usually been used as selection marker of recombinant bacteria and maintenance of recombinant plasmids vectors in E. coli and mycobacteria. However, the use of antibiotic resistance genes in strains aimed for therapy in humans prevents their use for commercial purposes. Furthermore, the presence of recombinant antibiotic resistances in microorganisms is limited to preclinical studies and Phase I clinical trials. Microorganisms must be re-designed and modified to eliminate antibiotic resistance. Furthermore, immunogenicity, efficacy and safety assays should be repeated, to ensure the complete bio-equivalence of the newly developed microorganism.

An alternative approach to the use of antibiotic selection markers in E. coli and mycobacteria is the use of genes necessary for the synthesis of a specific auxotrophic marker which allows cells auxotrophic for said marker to grow in minimal media in the absence of said auxotrophic marker. On the other hand, the approach auxotrophy/complementing gene could increase the plasmid stability in vitro and in vivo and could prevent the heterologous gene expression disruption by genetic rearrangements. Thus, the present invention addresses the need for Mycobacteria spp strains that are stable for clinical use and that may be useful for administering a polypeptide of interest to a subject in need thereof. The solution to this problem is attained by transforming a Mycobacteria spp strain cell, such as the Mycobacterium bovis BCG Pasteur strain cell, with a vector comprising the nucleotide sequence of the polypeptide of interest and wherein the strain carries an auxotrophy which is complemented by an auxotrophy complementing gene present forming part of the same vector as the vector which comprises the nucleotide sequence encoding a polypeptide of interest.

Thus, in a first aspect, the invention relates to a polynucleotide comprising:
(i) a sequence encoding a polypeptide of interest,
(ii) a first auxotrophy complementing gene which confers an auxotrophic host strain carrying said gene the capability of growing in a medium that lacks a first auxotrophic factor, and
(iii) a second auxotrophy complementing gene which confers an auxotrophic host strain carrying said gene the capability of growing in a medium that lacks a second auxotrophic factor.

In a second aspect, the invention relates to a polynucleotide comprising
(i) a sequence encoding a polypeptide of interest,
(ii) a mycobacterial origin of replication, and
(iii) at least one selection marker,
   wherein said polynucleotide does not comprise any nucleotide sequence conferring antibiotic sensitivity or antibiotic resistance to a cell carrying said polynucleotide.

In a further aspect, the invention refers to a vector comprising the polynucleotide of the invention.

In a further aspect, the invention relates to a recombinant cell comprising the polynucleotide or vector of the invention.

In a further aspect, the invention relates to a recombinant cell according to the invention for use in medicine and, in particular, for use in the treatment of a disease which requires the expression of a polypeptide of interest.

In another aspect, the invention relates to a vaccine composition comprising the bacterium according to the invention.

In a further aspect, the invention relates to a vaccine composition according to the invention for use in medicine.

In a further aspect, the invention relates to a vaccine composition according to the invention for use in inducing an immune response against an antigenic polypeptide.

Finally, the invention relates to a method for the expression of a polypeptide of interest in a Mycobacterium host cell which comprises:
(i) growing a Mycobacterium host cell comprising a sequence encoding the polypeptide of interest under the operative control of a weak mycobacterium promoter under conditions adequate for expression of the polypeptide of interest and, optionally,
(ii) recovering the polypeptide of interest from the culture.

### Deposit of Microorganisms

The plasmid p2auxo.HIVA and the bacterial strain BCG.HIVA^{2auxo} were deposited on Jul. 31, 2012 at Deutsche Sammlung vor Mikroorganismen und Zellculturen GmbH (DSMZ), Inhoffenstraβe 7 B, DE-38124, Braunschweig, Federal Republic of Germany and were assigned accession nos. DSM 26305 and DSM 26306, respectively.

### BRIEF DESCRIPTION OF THE FIGURES

FIG. 1. PCR of the αβT1 fragment corresponding to promoter+E. coli glyA gene+T1 termination sequence, flanked with SmaI-SpeI restriction enzyme target sites.

FIG. 2. SmaI digestion of the αβT1 PCR fragment and the pNEB193 vector.

FIG. 3. Transformation in E. coli of ligation product: αβT1 PCR fragment and pNEB193 vector. Obtention of pNEB193-αβT1 (L2-4 E. coli colony). A. E. coli colonies obtained after transformation of E. coli DH5-α with pNEB193-Sma1-αβT1 and selection in Amp-LB plates. B. E. coli colony identification: digestion of the plasmid DNA extracts with SmaI restriction enzyme. C. E. coli colony identification: digestion of the plasmid DNA extracts with SpeI restriction enzyme.

FIG. 4. pHIVACAT-1 parental plasmid DNA digested with SpeI, to release the kanamycin resistance gene, and purify the larger fragment for ligation (6815 bp).

FIG. 5. A. Ligation of p-HIVACAT-I (SpeI)+αβT1-SpeI(from pNEB193-αβT1). B. Selection of recombinant E. coli M15Δgly colonies in M9 agar plates (glycine defective)

FIG. 6. Construction of the BCG.HIVA^{2auxo} vaccine strain. A. A synthetic GC-rich HIVA gene was fused to the region encoding the 19-kDa lipoprotein signal sequence and inserted into the episomal pJH222.HIVA E. coli-mycobacterium shuttle plasmid. The BALB/c mouse T-cell and MAb Pk epitopes used in this work are depicted. P α-Ag, M. tuberculosis α-antigen promoter, PHSP60, heat shock protein 60 gene promoter. The aph gene was removed by SpeI digestion and the structural glyA gene was inserted and transformed into E. coli M15ΔGly strain. B. Immunodot of BCG.HIVA^{2auxo} lysates. Lanes 1-4: clones 1-4 of BCG.HIVA^{2auxo}. Lane 5: BCG wild type (negative control). Lysates of BCG.HIVA-GFP²²² were used as positive control. C. In vivo plasmid stability of BCG.HIVA^{auxo} harbouring p2auxo.HIVA. Mice were injected intradermically with 10⁶ cfu of BCG.HIVA^{2auxo} and boosted i.m. with 10⁶ pfu of MVA.HIVA. Spleens were homogenized 7 weeks after BCG inoculation and the recovered rBCG colonies were tested for the presence of the E. coli glyA and HIVA DNA coding sequence by PCR. Lanes 2-7 and 9-12: ten rBCG colonies were recovered in the non-lysine supplemented plate; lane 1 and 16: molecular weight marker lane 8: Plasmid DNA positive control (pQEαβT₁FucA and pJH222.HIVA plasmid DNA); lane 13 and 14: BCG wild type; lane 15: Distilled water (negative control).

FIG. 7. Genetic characterization of the BCG.HIVA^{2auxo} strain. A. The BCG.HIVA^{2auxo} Pasteur substrain identification by multiplex PCR assay. Lane 1 and 7: molecular weight marker (1 kb plus and 100 bp respectively, Invitrogen); lane 2: BCG.HIVA^{2auxo} Master Seed (MS); lane 3: BCG.HIVA^{2auxo} Working Vaccine Stock (WVS); lane 4: BCG wild type Pasteur substrain; lane 5: BCG Connaught substrain; lane 6: BCG Danish substrain. B. Enzymatic restriction analysis of p2auxo.HIVA plasmid DNA extracted from E. coli M15Δgly (pre-BCG transformation) and from both the MS and the WVS of BCG.HIVA^{2auxo} cultures. Left side: E. coli cultures. Lane 1 and 5: molecular weight marker (1 kb plus, Invitrogen); lane 2, 3 and 4: AgeI, Stu I and Xho I digestion, respectively. Right side: BCG cultures. Lane 9: molecular weight marker (1 kb plus, invitrogen); lane 6, 7, 8 (MS): AgeI, Stu I and Xho I digestion, respectively. Lane 10, 11 and 12 (WVS): AgeI, Stu I and Xho I digestion, respectively. C. PCR analysis of E. coli glyA DNA coding sequence using as template the cultures of BCG.HIVA^{2auxo} MS (lane 2), WVS (lane 3), p2auxo plasmid DNA without HIVA immunogen insert (lane 4), positive control plasmid DNA p2auxo.HIVA (lane 5), negative control, distilled water (lane 6) and molecular weight marker (lane 1). D. PCR analysis of HIVA DNA coding sequence using as template the cultures of BCG.HIVA^{2auxo} MS (lane 2), WVS (lane 3), positive control plasmid DNA p2auxo.HIVA (lane 4), negative control, distilled water (lane 5) and molecular weight marker (lane 1).

FIG. 8. Confirmation of positive E. coli M15 Δgly colony harboring the p2auxo.HIVA plasmid DNA (LL4-1).

FIG. 9. Transformation of p2auxo.HIVA into BCG lysA-strain. A. rBCG colonies obtained after transformation of BCG lysA-strain with p2auxo.HIVA and selection in lysine deficient medium. B. BCG Colony identification: PCR of HIVA and E. coli glyA DNA fragment.

FIG. 10. Phenotypic characterization of the E. coli M15 ΔglyA strain and BCG.HIVA^{2auxo} vaccine strain. The phenotype of glycine auxotrophy, glycine complementation and kanamycin resistance of E. coli M15ΔGly strain was assessed. The phenotype of lysine auxotrophy, lysine complementation and kanamycin resistance of BCG.HIVA^{2auxo} strain was assessed as well. A. E. coli glycine auxotroph strain plated on non-glycine supplemented M9-D agar plate. B. E. coli glycine auxotroph strain plated on glycine supplemented M9-D agar plate. C. E. coli M15ΔglyA strain harbouring the p2auxo.HIVA plasmid DNA and plated on M9-D agar plates without glycine and kanamycin supplementation. D. E. coli M15ΔglyA strain harbouring the p2auxo.HIVA plasmid DNA plated on M9-D agar plate without glycine supplementation and with kanamycin. E. BCG lysine auxotroph strain plated on non-lysine supplemented 7H10. F. BCG lysine auxotroph strain plated on lysine supplemented 7H10. G. BCG.HIVA^{2auxo} plated on 7H10 without lysine and kanamycin supplementation. H. BCG.HIVA^{2auxo} plated on 7H10 without lysine and with kanamycin supplementation.

FIG. 11. Schematic representation of the steps involved in the construction of p2auxo.HIVA plasmid DNA and BCG.HIVA^{2auxo} strain.

FIG. 12. p2auxo.HIVA plasmid DNA map.

FIG. 13. Induction of HIV-1- and Mtb-specific T-cells responses by the BCG.HIVA^{2auxo} prime-MVA.HIVA boost regimen. A. Adult mice (7-weeks-old) were either left unimmunized or primed with 10⁶ cfu of BCG.HIVA^{2auxo} or BCG wild type (intradermally), and boosted with 10⁶ pfu of MVA.HIVA (intramuscularly) 5 weeks post BCG inoculation. Mice were sacrificed 2 weeks later for T-cell analysis. B. Analysis of IFN-γ vaccine elicited HIV-1-specific CD8⁺ T-cell responses. The frequencies of cells producing cytokine are shown. Data are presented as means±SEM (n=8 for group 1, and n=5 for groups 2, 3 and 4). C. The functionality of vaccine-induced CD8⁺ T-cell responses was assessed in a multicolour intracellular cytokine staining assay. The group mean frequencies of single-, double- or triple-cytokine-producing P18110-specific cells are shown for the four vaccination groups. D. Elicitation of specific T-cell responses was assessed in an ex vivo IFN-γ ELISPOT assay using the immunodominant P18I10 CD8⁺ T-cell epitope peptide. The median spot-forming units (SFU) per 10⁶ splenocytes for each group of mice (n=8 for group 1, and n=5 for groups 2, 3 and 4) as well as individual animal responses is shown. E. PPD-specific T-cell responses elicited by BCG.HIVA^{2auxo}. Immune responses to BCG were assessed in an ex vivo IFN-γ ELISPOT assay using PPD as the antigen. The median spot-forming units (SFU) per 10⁶ splenocytes for each group of mice (n=8 for group 1, and n=5 for groups 2, 3 and 4) as well as individual animal responses is shown. *=p<0.05, **=p<0.01.

FIG. 14. BCG.HIVA^{2auxo} prime and MVA.HIVA boost safety in adult mice. A. Adult mice were either left unimmunized or immunized with 10⁶ cfu of BCG wild type or BCG.HIVA^{2auxo} by intradermal route and subsequently given a booster dose of 10⁶ pfu of MVA.HIVA at week 5 by intramuscular route. B. The body weight was weekly recorded, and the mean for each group of mice is shown (n=10 for group 1 and n=5 for groups 2, 3 and 4). Data from naive mice are presented as mean±standard deviation (SD, n=5). The weight differences between vaccinated and naïve mice group were analyzed weekly by ANOVA test.

FIG. 15. A. p2auxo.CSP plasmid DNA map. CSP: Circumsporozoite protein from Plasmodium berghei. First, the plasmid DNA p2auxo.HIVA was digested by HindIII restriction enzyme, and the HIVA DNA coding sequence was released. Second, the CSP immunogen DNA coding sequence, was amplified by PCR using specific primers and HindIII extension sites and was inserted into p2auxo vector.

FIG. 16. A. p2auxo.Ag85B plasmid DNA map. Ag85B from Mycobacterium bovis. First, the plasmid DNA p2auxo.HIVA was digested by HindIII restriction enzyme, and the HIVA DNA coding sequence was released. Second, the Ag85B immunogen DNA coding sequence, was amplified by PCR using specific primers and HindIII extension sites and was inserted into p2auxo vector.

FIG. 17. A. p2auxo.HIVc(G+C) plasmid DNA map. The HIVc DNA sequence was BCG codon optimized and synthesized in vitro and cloned into pGH plasmid DNA (Biomatik, USA). First, the plasmid DNA p2auxo.HIVA was digested by HindIII restriction enzyme, and the HIVA DNA coding sequence was released. Second, the HIVc immunogen DNA coding sequence was released by HindIII digestion and inserted into p2auxo vector.

### DETAILED DESCRIPTION OF THE INVENTION

### 1. Definitions of General Terms and Expressions

The term “α-antigen promoter”, as used herein, refers to the promoter region of the gene encoding a mycobacterium α-antigen and corresponds to a polynucleotide comprising the minimal region of the upstream region of the α-antigen gene which is suitable for efficient promoter activity as in SEQ ID NO:027 for Mycobacterium tuberculosis and equivalent regions in the α-antigen gene of other mycobacteria (e.g. M. stegmatis). The term α-antigen, also known as Ag 85B, Ag 6 and MPT59 is one of the most dominant secretory proteins and is a major stimulant of cellular and humoral immunity. It is widely distributed among M. tuberculosis, Bacillus Calmette-Guérin (BCG) isolated from Mycobacterium bovis, and atypical mycobacteria. This Ag belongs to the Ag 85 complex, which consists of three structurally related components, Ag 85A, Ag 85B (α-Ag), and Ag 85C.

The term “adjuvant”, as used herein, refers to a substance which, when added to an immunogenic agent, nonspecifically enhances or potentiates an immune response to the agent in a recipient host upon exposure to the mixture. Examples of adjuvants include, but are not limited to, agonistic antibodies to co-stimulatory molecules, Freund's adjuvant, muramyl dipeptides, or liposomes.

The term “administration”, as used herein, refers to routes of introducing the recombinant bacterium of the invention to a subject. Examples of routes of administration which can be used include injection (e.g. intradermal, subcutaneous, intravenous, parenterally, intraperitoneally, intrathecally), intravesically (e.g. urinary bladder), intraprostatically, oral, inhalation, rectal and transdermal. The pharmaceutical preparations are of course given by forms suitable for each administration route. For instance, these preparations are administered in tablets or capsule form, by injection, inhalation, eye lotion, ointment or suppository. The injection can be bolus or can be by continuous infusion. The recombinant bacterium can be administered alone, or in conjunction with either another agent as described above or with a pharmaceutically acceptable carrier, or both. The recombinant bacterium can be administered prior to the administration of the other agent, simultaneously with the agent, or after the administration of the agent. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. The terms “systemic administration”, “administered systemically”, “peripheral administration” and “administered peripherally” as used herein mean the administration of the recombinant bacterium, such that it enters the patient's system and, thus, is subject to metabolism and other like processes.

The term “AIDS”, as used herein, refers to the symptomatic phase of HIV infection, and includes both Acquired Immune Deficiency Syndrome (commonly known as AIDS) and “ARC,” or AIDS-Related Complex. See Adler M, et al., Brit. Med. J. 1987; 294: 1145-1147. The immunological and clinical manifestations of AIDS are known in the art and include, for example, opportunistic infections and cancers resulting from immune deficiency.

The term “antibiotic”, as used herein, refers to a chemical substance produced by a living being or a synthetic derivative thereof which at low concentrations kills or prevents the growth of certain classes of sensitive microorganisms, generally bacteria, although some antibiotics are also used for the treatment of infections by fungi or protozoa. Antibiotics are used in human, animal or horticultural medicine to treat infections caused by microorganisms. Antibiotics included in the present invention are, without limitation, aminoglycoside antibiotics, ansamycins, carbacefem, carbapenems, cephalosporins, glycopeptides, macrolides, monobactams, penicillins, polypeptides, quinolones, sulfonamides, tetracyclines and others such as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin or rifampicin, tinidazole, viomycin and capreomycin; preferably cephalosporins, tetracyclines, glycopeptides, carbapenems, polypeptides, rifampicin, aminoglycosides, sulfonamides, viomycin and capreomycin. In a preferred embodiment the antibiotic is selected from the group of aminoglycosides and cephalosporins.

The term “antigen”, as used herein, refers to any molecule or molecular fragment that, when introduced into the body, induces a specific immune response (i.e. humoral or cellular) by the immune system. Antigens have the ability to be bound at the antigen-binding site of an antibody.

The term “Ag85B”, as used herein, relates to a Mycobacterium antigen protein. In a particular embodiment, the term Ag85B relates to the extracellular alpha antigen Ag85B of Mycobacterium bovis, more particularly of Mycobacterium bovis BCG, which is one of the major secreted proteins from M. bovis BCG. The cloning and expression of the Mycobacterium bovis BCG gene for extracellular alpha antigen was described by Matsuo K et al. 1998 J Bacteriol 170(9): 3847-3854. The sequence corresponding to Mycobacterium bovis gene for alpha antigen (antigen 85B) is located at NCBI database under accession number D78142.1 (738 pb, release as of 1 February 2000). In a particular embodiment, the nucleotide sequence of Ag85B is the sequence SEQ ID NO:032.

The term “antibiotic resistance”, as used herein, relates to the ability of a microorganism, particularly a bacterium, to survive in the presence of or after the exposure to, an antibiotic.

The term “antibiotic sensitivity”, as used herein, relates to the susceptibility of a microorganism, particularly a bacterium, to an antibiotic. Methods to determine antibiotic sensitivity of a bacterium are known in the art and include the Kirby-Bauer method and the Stokes method.

The term “auxotrophy”, as used herein, refers to a requirement of one or more specific substances for growth and metabolism in an organism due to an inability to synthesize said substance(s) and that the parental organism was able to synthesize.

The terms “auxotrophy gene” and “auxotrophy-complementing gene”, as used interchangeably herein, refer to a gene which is involved in the synthesis of the auxotrophic factor and which allows a host which is auxotrophic for said factor to grow in the absence of said factor.

The term “auxotrophic host”, as used herein, refers to a microorganism having a specific nutritional requirement for said factor (the “auxotrophic factor” which may be, for instance, an amino acid or a sugar) and which is usually not required by the wild-type organism resulting in that the microorganism is not capable of growing or replicating without supplementation by said factor.

The term “Bacillus Calmette-Guérin” or “BCG”, as used herein, refers to a vaccine against tuberculosis that is prepared from a strain of the attenuated (weakened) live Mycobacterium bovis that has lost its virulence in humans by being specially subcultured (230 passages) in an artificial medium for 13 years. Suitable BCG includes, without limitation, wild-type BCG such as BCG strains BCG₁₃₃₁, BCG Pasteur, BCG Tokyo, BCG Copenhagen, BCG Moreau or BCG Moscow. Moreover, the term BCG also refers to a recombinant BCG (rBCG).

The term “boosting vaccine” as used herein, refers to a vaccine comprising an agent that encodes an antigen that has an immunologically active portion of the target antigen, and may include the target antigen, be a fragment thereof, or be a fusion polypeptide containing at least an immunologically active portion of the target antigen joined to a region that is not normally present in the target antigen.

The term “carrier”, as used herein, relates to an immunogenic molecule which, when bound to a second molecule, augments immune responses to the latter.

The term “cell”, as used herein, is equivalent to “host cell” and is intended to refer to a cell into which a viral genome, a vector, a plasmid DNA, or a HIV-1 viral particle of the invention has been introduced. It should be understood that such terms refer not only to the particular subject cell but to the progeny, or potential progeny, of such a cell. Because certain modifications may occur in succeeding generations due to either mutation or environmental influences, such progeny may not, in fact, be identical to the parent cell, but can be still included within the scope of the term as used herein.

The term “comprising” or “comprises”, as used herein, discloses also “consisting of” according to the generally accepted patent practice.

The term “CSP”, as used herein, relates to Plasmodium circumsporozoite protein. In a particular embodiment, the CSP is the Plasmodium berghei CSP. Plasmodium berghei circumsporozoite protein (CSP) gene sequence is located at NCBI GenBank database under accession number M28887.1 (999 pb, release as of 26 Apr. 1993). The sequence of the circumsporozoite gene of Plasmodium berghei ANKA clone and NK65 strain was described in Lanar D E 1990 Mol Biochem Parasitol 39(1): 151-153. In a particular embodiment, the nucleotide sequence of CSP is the sequence SEQ ID NO: 033. The expression “disease associated with a HIV infection”, as used herein, includes a state in which the subject has developed AIDS, but also includes a state in which the subject infected with HIV has not shown any sign or symptom of the disease.

The expression “disease associated with a mycobacteria infection”, as used herein, refers to any disease resulting from the infection by any mycobacteria, including tuberculosis and leprosy. The term “tuberculosis” comprises infections due to Mycobacterium tuberculosis and Mycobacterium bovis; respiratory tuberculosis such as tuberculosis of lung, larynx, trachea and bronchus, tuberculosis of intrathoracic lymph nodes, tuberculous pleurisy, primary respiratory tuberculosis and other respiratory tuberculosis; tuberculosis of the nervous system such as tuberculous meningitis, tuberculosis of meninges, tuberculous leptomeningitis, meningeal tuberculoma and other tuberculosis of nervous system; tuberculosis of bones and joints, tuberculosis of genitourinary system, tuberculous peripheral lymphadenopathy, tuberculosis of intestines, peritoneum and mesenteric glands, tuberculosis of skin and subcutaneous tissue, tuberculosis of eye, ear, or adrenal glands, and miliary tuberculosis (International Classification of Diseases, 10th Revision, Blocks A15-A19). The term ‘leprosy’ (Hansen's disease) comprises infections caused by Mycobacterium leprae; indeterminate leprosy, tuberculoid leprosy, borderline leprosy, borderline tuberculoid leprosy, lepromatous leprosy and other forms of leprosy (international Classification of Diseases, 10th Revision, Block A30).

The term “E. coli/mycobacterial shuttle vector”, as used herein, refers to a plasmid DNA vector which is capable of replication in E. coli and in mycobacteria.

The term “endosomalytic peptide”, as used herein, refers to a peptide which is capable of mediating endosomal escape of molecules which incorporate the endosomalytic peptide (i.e. peptides with the ability to degrade the endosome), either partially resulting in leakage of antigens into the cytoplasm, or to the extent that the endosome is ruptured and the mycobacterium strain escapes this subcellular compartment and resides in the cytoplasm. See Hess J, et al., Proc. Natl. Acad. Sci. USA 1998; 95:5299-5304 and Grode L, et al., Clin. Invest. 2005; 115:2472-2479.

The term “epitope”, as used herein, refers to that portion of a given immunogenic substance that is the target of or is bound by an antibody or a cell-surface receptor of a host immune system that has mounted an immune response to the given immunogenic substance as determined by any method known in the art. Further, an epitope may be defined as a portion of an immunogenic substance that elicits an antibody response or induces a T-cell response in an animal, as determined by any method available in the art. See Walker J, Ed., “The Protein Protocols Handbook” (Humana Press, Inc., Totoma, N.J., US, 1996). An epitope can be a portion of any immunogenic substance (e.g. a protein, polynucleotide, polysaccharide, an organic or inorganic chemical) or any combination thereof. The term “epitope” may also be used interchangeably with “antigenic determinant” or “antigenic determinant site”.

The term “glyA” or “glyA gene”, as used herein, refers to the gene encoding serine hydroxymethyl transferase (SHMT), an enzyme involved in the main glycine biosynthesis pathway and can be used in an E. coli host carrying a deletion in the glyA gene. See Vidal L, et al., J. Biotechnol. 2008; 134:127-136.

The term “heterologous promoter”, as used herein, refers to a promoter which is operably linked to a polynucleotide encoding the polypeptide of interest but wherein (i) the promoter is not naturally associated to the operably linked gene encoding the polypeptide of interest or ii) the promoter is naturally associated to the operably linked encoding the polypeptide of interest but its sequence has been modified from its original form.

The term “HIVA”, as used herein, refers to a fusion polypeptide derived from the sequences of HIV-1 clade A formed by a fragment of the gag protein fused and a string of 25 partially overlapping CTL epitopes. See Hanke T, et al., Nat. Med. 2000; 6:951-955. The gag domain of HIVA contains p24 and p17 in an order reversed to the viral gag p17-p24-p15 polyprotein. This rearrangement prevents myristylation of the N-terminus of p17, which could direct the recombinant protein to the cell membrane, thus preventing efficient degradation into peptides necessary for the major histocompatibility complex (MHC) class I presentation. The C-terminus of the HIVA protein contains a multi-CTL epitope synthetic polypeptide which is 8- to 10-amino acids long and originates from the gag, pol, nef or env proteins. See Rowland-Jones S., et al., J. Clin. Invest. 1998; 102:1758-1765 and Dorrell L, et al., J. Virol. 1999; 73:1708-1714. Many of these epitopes are immunodominant and relatively conserved among other HIV-1 clades and, therefore, should be able to elicit an immune response which cross-reacts with HIV viruses of clades other than clade A. See Table 1. They are presented by seventeen different HLA alleles, which include both frequent African alleles as well as alleles common in most ethnic populations. The HIVA synthetic polypeptide also comprises SIV gag and HIV env epitopes recognized by macaque and murine CTL, respectively, so that the quality, reproducibility and stability of the clinical batches could easily be assessed in a mouse (or macaque if necessary) potency assay. A monoclonal antibody epitope Pk was added to the C-terminus of HIVA for easy detection of the full-size protein and estimation of the level of expression. See Hanke T, et al., J. Gen. Virol. 1992; 73:653-660.

TABLE 1
HIVA immunogen epitopes

| Epitope^{a} | MHC class I restriction | Origin | HIV clade^{b} | SEQ ID NO: |
|---|---|---|---|---|
| ALKHRAYEL | HLA-A*0201 | nef | a | 001 |
| PPIPVGEIY | HLA-B35 | p24 | a/B/c/D/F/G | 002 |
| GEIYKRWII | HLA-B8 | p24 | a/B/c/D/F/G | 003 |
| KRWIILGLNK | HLA-B*2705 | p24 | A/B/C/D/F/G/H | 004 |
| FRDYVDRFYK | HLA-B18 | p24 | BD(A = C/F/G/H)^{b} | 005 |
| RDYVDRFYKTL | HLA-B44 | P24 | B/D(A = C/F/G/H)^{c} | 006 |
| DRFYKTLRA | HLA-B14 | p24 | B/D(A = C/F/G/H) | 007 |
| ALFQSSMTK | HLA-A*0301, A11, A33 | po1 | aB/c/D/G/H | 008 |
| ITLWQRPLV | HLA-A*6802 | po1 | a/b/C/D/F/G/H | 009 |
| ERYLKDQQL | HLA-B14 | gp41 | a/b/C/D | 010 |
| YLKDQQLL | HLA-A24, B8 | gp41 | a/b/C/D | 011 |
| TVYYGVPVWK | HLA-A*0301 | gp120 | AB/C/D/g | 012 |
| RPQVPLRPMTY | HLA-B51 | nef | A/b/D/E/F/G | 013 |
| QVPLRPMTYK | HLA-A*0301, A11 | nef | A/b/D/E/F/G | 014 |
| VPLRPMTY | HLA-B35 | nef | A/b/D/E/F/G | 015 |
| AVDLSHFLK | HLA-A1 1 | nef | aB/d/f | 016 |
| DLSHFLKEK | HLA-A*0301 | nef | AB/D/F | 017 |
| FLKEKGGL | HLA-B8 | nef | AB/C/D/E/F/G | 018 |
| ILKEPVHGV | HLA-A*0201 | po1 | AB/C/D/G | 019 |
| ILKEPVHGVY | HLA-Bw62 | po1 | A/B/D | 020 |
| HPDIVIYQY | HLA-B35 | po1 | a | 021 |
| VIYQYMDDL | HLA-A*0201 | po1 | AB/C/D/F/G/H | 022 |
| TPGPGVRYPL | HLA-B7 | nef | b/c | 023 |
| ACTPYDINQML^{d} | Mamu-A*01 | p27 | SIV | 024 |
| RGPGRAFVTI^{e} | H-2D^{d} | env | HIV | 025 |

Note:
Epitopes are listed (Seq. ID Nos. 1-25) in the order in which they appear in the polyepitope,
^{b}A particular epitope sequence is present in about 50 percent (small letter) or 90 percent (capital letter) of sequenced HIV clade isolates,
^{c}‘=’ indicates that the epitopes are present in the N-terminal clade A gag domain,
^{d}A dominant epitope derived from SIV gag p27 flanked by Ala and Leu at its N- and C-termini, respectively, recognized by rhesus macaque CTL, which can be used for potency studies in rhesus macaques,
^{e}A CTL epitope presented by a murine MHC class I used for the mouse potency assay.

The term “HIV”, as used herein, include HIV-1 and HIV-2 and SW. “HIV-1” means the human immunodeficiency virus type-1. HIV-1 includes, but is not limited to, extracellular virus particles and the forms of HIV-1 associated with HIV-1 infected cells. The HIV-1 virus may represent any of the known major subtypes (Classes A, B, C, D E, F, G and H) or outlying subtype (Group O) including laboratory strains and primary isolates. “HIV-2” means the human immunodeficiency virus type-2. HIV-2 includes, but is not limited to, extracellular virus particles and the forms of HIV-2 associated with HIV-2 infected cells. The term “SIV” refers to simian immunodeficiency virus which is an HIV-like virus that infects monkeys, chimpanzees, and other nonhuman primates. SIV includes, but is not limited to, extracellular virus particles and the forms of SIV associated with SIV infected cells.

The term “HIV immunogen”, as used herein, refers to a protein or peptide antigen derived from HIV that is capable of generating an immune response in a subject and also refers to a HIV viral particle, being said particle a whole viral particle or a viral particle lacking one or more viral components but retaining the ability to generate an immune response. HIV immunogens for use according to the present invention may be selected from any HIV isolate (e.g. any primary or cultured HIV-1, HIV-2, or isolate, strain, or clade). HIV isolates are now classified into discrete genetic subtypes. HIV-1 is known to comprise at least ten subtypes (A1, A2, A3, A4, B, C, D, E, PL F2, G, H, J and K). See Taylor B, et al., New Engl. J. Med 2008; 359(18):1965-1966. HIV-2 is known to include at least five subtypes (A, B, C, D, and E). HIV-1 and HIV-2 have been associated with the HIV epidemic in homosexual men and intravenous drug users worldwide. Most HIV-1 immunogens, laboratory adapted isolates, reagents and mapped epitopes belong to subtype B. In sub-Saharan Africa, India, and China, areas where the incidence of new HIV infections is high, HIV-1 subtype B accounts for only a small minority of infections, and subtype HIV-1 subtype C appears to be the most common infecting subtype. Thus, in certain embodiments, it may be preferable to select immunogens from particular subtypes (e.g. HIV-1 subtypes B or C). It may be desirable to include immunogens from multiple HIV subtypes (e.g. HIV-1 subtypes B and C, HIV-2 subtypes A and B, or a combination of HIV-1 or HIV-2) in a single immunological composition. Suitable HIV immunogens includes gag, pol, env, vif, vpr, tat, rev, nef, or vpu as well as fragments thereof having at least 6 amino acids in length, and can be, for example, at least 8, at least 10, at least 14, at least 16, at least 17, at least 18, at least 19, at least 20 or at least 25 amino acids or greater.

The term “HIV-1 viral particle”, as used herein, refers to a roughly spherical structure with a diameter of about 120 nm composed of two copies of positive single-stranded RNA that encodes the virus nine genes enclosed by a conical capsid composed of 2,000 copies of the viral protein p24. The single-stranded RNA is tightly bound to nucleocapsid proteins, p7, and enzymes needed for the development of the virion such as reverse transcriptase, proteases, ribonuclease and integrase. A matrix composed of the viral protein p17 surrounds the capsid ensuring the integrity of the virion particle. This is, in turn, surrounded by the viral envelope that is composed of two layers of fatty molecules called phospholipids taken from the membrane of a human cell when a newly formed virus particle buds from the cell. Embedded in the viral envelope are proteins from the host cell and about 70 copies of a complex HIV protein that protrudes through the surface of the virus particle. This protein, known as Env, consists of a cap made of three molecules called glycoprotein (gp) 120, and a stem consisting of three gp41 molecules that anchor the structure into the viral envelope. This glycoprotein complex enables the virus to attach to and fuse with target cells to initiate the infectious cycle.

The term “host cell”, as used herein, refers to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “immune response”, as used herein, includes any response associated with immunity including, but not limited to, increases or decreases in cytokine expression, production or secretion (e.g. IL-12, IL-10, TGF beta or TNF□ expression, production or secretion), cytotoxicity, immune cell migration, antibody production or immune cellular responses. The phrase “modulating an immune response” or “modulation of an immune response” includes upregulation, potentiating, stimulating, enhancing or increasing an immune response, as defined herein. For instance, an immune response can be upregulated, enhanced, stimulated or increased directly by use of a modulator of the present invention (e.g. a stimulatory modulator). Alternatively, a modulator can be used to “potentiate” an immune response, for example, by enhancing, stimulating or increasing immune responsiveness to a stimulatory modulator. The phrase “modulating an immune response” or “modulation of an immune response” also includes downregulation, inhibition or decreasing an immune response as defined herein. Immune responses in a subject or patient can be further characterized as being either type-1 or type-2 immune responses. A “type-1 immune response”, also referred to herein as a “type-1 response” or a “T helper type 1 (Th1) response” includes a response by CD4+ T cells that is characterized by the expression, production or secretion of one or more type-1 cytokines and that is associated with delayed type hypersensitivity responses. The phrase “type-1 cytokine” includes a cytokine that is preferentially or exclusively expressed, produced or secreted by a Th1 cell, that favors development of Th1 cells or that potentiates, enhances or otherwise mediates delayed type hypersensitivity reactions. Preferred type-1 cytokines include, but are not limited to, GM-CSF, IL-2, IFN-γ, TNF-a, IL-12, IL-15 and IL-18. A “type-2 immune response”, also referred to herein as a “type-2 response” or a “T helper type 2 (Th2) response” refers to a response by CD4+ T cells that is characterized by the production of one or more type-2 cytokines and that is associated with humoral or antibody-mediated immunity (e.g. efficient B cell, “help” provided by Th2 cells, for example, leading to enhanced IgG1 or IgE production). The phrase “type-2 cytokine” includes a cytokine that is preferentially or exclusively expressed, produced or secreted by a Th2 cell, that favours development of Th2 cells or that potentiates, enhances or otherwise mediates antibody production by B lymphocytes. Preferred type-2 cytokines include, but are not limited to, IL-4, IL-5, IL-6, IL-10, and IL-13.

The terms “immunize”, “immunization” or their equivalents, as used herein, refer to conferring the ability to mount a substantial immune response against a target antigen or epitope. The antigen or epitope may be isolated or expressed by a microbe. These terms do not necessarily require complete immunity, but rather that an immune response could be produced that is substantially greater than the baseline response (e.g. where immunogenic compositions of the invention are not administered or where a conventional vaccine is administered). For example, a mammal is considered to be immunized against a target antigen, if the cellular or humoral immune response, preferably a substantial response, to the target antigen occurs following the application of compositions of the invention or according to methods of the invention. Generally, an immunological response includes, but is not restricted to, one or more of the following effects: (a) the production of antibodies; (b) the production of B cells; (c) the production of helper T cells; or (d) the production of cytotoxic T cells, that are specifically directed to a given antigen or hapten.

The term “immunogen”, as used herein, refers to an antigen capable of provoking an adaptive immune response if administered alone. All immunogens are also antigens but not all antigens are immunogens. In a particular embodiment, the immunogenic polypeptide belongs to bacteria, viruses, parasites and other microorganisms such as coats, capsules, cell walls, flagella, fimbriae and toxins. Examples of antigens according to the present invention include antigens from picornavirus, coronavirus, togavirus, flavirvirus, rhabdovirus, paramyxovirus, orthomyxovirus, bunyavirus, arenavirus, reovirus, retrovirus, papillomavirus, parvovirus, herpesvirus, poxvirus, hepadnavirus, and spongiform virus families; or from other pathogens such as trypanosomes, tapeworms, roundworms, helminthes or malaria. Examples of suitable viral antigens are, without limitation: retroviral antigens from the human immunodeficiency virus (HIV) including gene products of the gag, pol, env and nef genes, and other HIV components; hepatitis viral antigens, such as the S, M, and L proteins of hepatitis B virus, the pre-S antigen of hepatitis B virus, and other hepatitis (e.g. hepatitis A, B, and C, viral components such as hepatitis C viral RNA); influenza viral antigens, such as hemagglutinin and neuraminidase and other influenza viral components; measles viral antigens, such as the measles virus fusion protein and other measles virus components; rubella viral antigens, such as proteins E1 and E2 and other rubella virus components; rotaviral antigens, such as VP7sc and other rotaviral components; cytomegaloviral antigens, such as envelope glycoprotein B and other cytomegaloviral antigen components; respiratory syncytial viral antigens, such as the RSV fusion protein, the M2 protein and other respiratory syncytial viral antigen components; herpes simplex viral antigens, such as immediate early proteins, glycoprotein D, and other herpes simplex viral antigen components; varicella zoster viral antigens, such as gpI, gpII, and other varicella zoster viral antigen components; Japanese encephalitis viral antigens, such as proteins E, M-E, M-E-NS1, NS1, NS1-NS2A, 80 percent E, and other Japanese encephalitis viral antigen components; rabies viral antigens, such as rabies glycoprotein, rabies nucleoprotein and other rabies viral antigen components. See Fields B, et al., Eds., “Fundamental Virology”, 2^{nd} Ed. (Raven Press, New York, N.Y., US, 1991). The term “antigenic polypeptide” or an “immunogenic polypeptide”, as used herein, refers to a polypeptide which, when introduced into a vertebrate, reacts with the vertebrate's immune system molecules, is antigenic, or induces an immune response in the vertebrate (i.e. is immunogenic).

The term “immunogenic composition”, as used herein, refers to a composition that elicits an immune response in a subject that produces antibodies or cell-mediated immune responses against a specific immunogen. The term “antigenic composition” refers to a composition that can be recognized by a host immune system. For example, an antigenic composition contains epitopes that can be recognized by humoral or cellular components of a host immune system.

The term “inactivated HIV virus”, as used herein, refers to an intact, inactivated HIV virus. An inactivated HIV refers to a virus that cannot infect or replicate. A whole inactivated HIV virus generally maintains native structure of viral antigens to maintain immunogenicity and stimulate immune responses to native virus.

The term “lysA5” or “lysA5 gene”, as used herein, refers to a gene encoding diaminopimelic acid decarboxylase which catalyzes the conversion of diaminopimelic acid (DAP) to lysine and can be used in a mycobacterial host carrying a deletion in the lysA gene such as the Mycobacteria bovis BCG Δlys5::res. See Pavelka M, et al., J. Bacteriol. 1999; 181:4780-4789).

The term “medicament”, as used herein, is understood to be a pharmaceutical composition, and particularly, a vaccine or immunogenic composition of the invention.

The term “modified vaccinia virus Ankara” or “MVA”, as used herein, refers to a strain of vaccinia virus which does not replicate in most cell types, including normal human tissues and which was derived by serial passage greater than 500 times in chick embryo fibroblasts (CEF) of material derived from a pox lesion on a horse in Ankara, Turkey. See Mayr A, et al., Infection 1975; 33:6-14. Typically, the nucleic acid encoding the antigenic peptide is incorporated into a non-essential region of the genome of the MVA. According to the present invention, any attenuated vaccinia virus strain can be used. Non-essential regions may be selected from (i) natural occurring deletion sides of the MVA genome with respect to the genome of the vaccinia virus strain Copenhagen or (N) intergenic regions of the MVA genome. The term “intergenic region” refers preferably to those parts of the viral genome located between two adjacent genes that comprise neither coding nor regulatory sequences. However, the insertion sides for the incorporation of the nucleic acid encoding the antigenic polypeptide are not restricted to these preferred insertion sides since it is within the scope of the present invention that the integration may be anywhere in the viral genome as long as it is possible to obtain recombinants that can be amplified and propagated in at least one cell culture system, like chicken embryo fibroblasts (CEF cells). Thus, a non-essential region may also be a non-essential gene or genes, the functions of which may be supplemented by the cell system used for propagation of MVA.

The term “mycobacteria”, as used herein, refers to any Gram-positive bacteria belonging to the Mycobacterium genus. Suitable members of the mycobacteria include, but are not limited to, Mycobacterium tuberculosis, Mycobacterium leprae, Mycobacterium avium intracellulare, Mycobacterium kansasii, and Mycobacterium ulcerans.

The term “operably linked”, as used herein, is intended to mean that the nucleotide sequence of interest is linked to the regulatory sequence(s) in a manner that allows for expression of the nucleotide sequence (e.g. in an in vitro transcription/translation system or in a host cell when the vector is introduced into the host cell). See Auer H, Nature Biotechnol. 2006; 24: 41-43.

The terms “pharmaceutically acceptable carrier,” “pharmaceutically acceptable diluent,” “pharmaceutically acceptable excipient”, or “pharmaceutically acceptable vehicle”, used interchangeably herein, refer to a non-toxic solid, semisolid or liquid filler, diluent, encapsulating material or formulation auxiliary of any conventional type. A pharmaceutically acceptable carrier is essentially non-toxic to recipients at the employed dosages and concentrations and is compatible with other ingredients of the formulation. The number and the nature of the pharmaceutically acceptable carriers depend on the desired administration form. The pharmaceutically acceptable carriers are known and may be prepared by methods well known in the art. See Faulí i Trillo C, “Tratado de Farmacia Galénica” (Ed. Luzán 5, S. A., Madrid, ES, 1993) and Gennaro A, Ed., “Remington: The Science and Practice of Pharmacy” 20th Ed. (Lippincott Williams & Wilkins, Philadelphia, Pa., US, 2003). They are involved in carrying or transporting the subject chemical from one organ, or portion of the body, to another organ, or portion of the body without excessive toxicity, irritation, allergic response, or other problem or complication, commensurate with a reasonable benefit/risk ratio. Each carrier must be “acceptable” in the sense of being compatible with the other ingredients of the formulation and not injurious to the patient. Some examples of materials which can serve as pharmaceutically-acceptable carriers include: (a) sugars (e.g. lactose, glucose and sucrose), (b) starches (e.g. corn starch and potato starch), (c) cellulose and its derivatives (e.g. sodium carboxymethyl cellulose, ethyl cellulose and cellulose acetate), (d) powdered tragacanth, (e) malt, (f) gelatin, (g) talc, (h) excipients (e.g. cocoa butter and suppository waxes), (i) oils (e.g. peanut oil, cottonseed oil, safflower oil, sesame oil, olive oil, corn oil and soybean oil), (j) glycols (e.g. propylene glycol), (k) polyols (e.g. glycerin, sorbitol, mannitol and polyethylene glycol), (1) esters (e.g. ethyl oleate and ethyl laurate), (m) agar, (n) buffering agents (e.g. magnesium hydroxide and aluminum hydroxide), (o) alginic acid, (p) pyrogen-free water, (q) isotonic saline, (r) Ringer's solution, (s) ethyl alcohol, (t) phosphate buffer solutions and (u) other non-toxic compatible substances employed in pharmaceutical formulations. Wetting agents, emulsifiers and lubricants (e.g. sodium lauryl sulfate and magnesium stearate), as well as coloring agents, release agents, coating agents, sweetening, flavoring and perfuming agents, preservatives and antioxidants can also be present in the compositions. Examples of pharmaceutically-acceptable antioxidants include: (a) water soluble antioxidants (e.g. ascorbic acid, cysteine hydrochloride, sodium bisulfate, sodium metabisulfite or sodium sulfite), (b) oil-soluble antioxidants (e.g. ascorbyl palmitate, butylated hydroxyanisole (BHA), butylated hydroxytoluene (BHT), lecithin, propyl gallate or α-tocopherol), and (c) metal chelating agents (e.g. citric acid, ethylenediamine tetraacetic acid (EDTA), sorbitol, tartaric acid or phosphoric acid).

The term “pharmaceutical composition”, as used herein, refers to a composition comprising at least one recombinant bacterial strain of the invention. The pharmaceutical composition can be immunogenic or antigenic. Pharmaceutical compositions may include at least one pharmaceutically acceptable carrier and can be prepared, for instance, as injectables such as liquid solutions, suspensions, and emulsions.

The terms “plasmid DNA vector” and “expression vector”, as used herein, refer to a nucleic acid molecule capable of transporting another nucleic acid to which it has been linked. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, like viral vectors (e.g. replication defective retroviruses, adenoviruses and adeno-associated viruses), which serve equivalent functions.

The terms “polynucleotide” and “nucleotide sequence”, as used interchangeably herein, relate to any polymeric form of nucleotides of any length and composed of ribonucleotides or deoxyribonucleotides. The terms include both single-stranded and double-stranded polynucleotides, as well as modified polynucleotides (e.g. methylated, protected). Typically, the polynucleotide is a “coding sequence”, which, as used herein, refers to a DNA sequence which is transcribed and translated into a polypeptide in a host cell when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g. mammalian) DNA, and even synthetic DNA sequences. A transcription termination sequence will usually be located 3′ to the coding sequence.

The term “polypeptide of interest”, as used herein, refers to any polypeptide that is desirably introduced or expressed in a subject. Suitable polypeptides of interest that can be encoded by the polynucleotides of the invention include, without limitation, transcription factors, receptors, enzymes, structural proteins, cytokines, cytokine receptors, lectins, selectins, immunoglobulins, kinases, phosphatases, prions, proangiogenic polypeptides, proteases and proteins involved in the apoptosis process, adhesion molecules, surface receptors, proteins involved in metastasis or in invasive processes of tumor cells, growth factors, the multiple drug resistance gene (MDR1), lymphokines, cytokines, immunoglobulins, T-cell receptors, MHC antigens, DNA and RNA polymerases, proteins involved in metabolic processes such as the synthesis of amino acids, nucleic acids, tumor suppressing genes, 5-lipoxygenase, phospholipase A2, protein kinase C, p53, p16, p21, MMAC1, p73, zac1, C-CAM, BRCA1, Rb, Harakiri, Ad E1B, protease ICE-CED3, IL-2, IL-3, IL-4, IL-5, IL-6, IL-7, IL-8, IL-9, IL-10, IL-11, IL-12, IL-13, IL-14, IL-15, TNF, GMCSF, interferons, CFTR, EGFR, VEGFR, IL-2 receptor, estrogen receptor, members of the Bcl-2 family (Bcl-2 or Bcl-xL), ras, myc, neu, raf erb, src, fins, jun, trk, ret, gsp, hst and abl, amyloid protein precursor, angiostatin, endostatin, METH-1, METH-2, Factor IX, Factor VIII, collagen, cyclin dependent kinase, cyclin D1, cyclin E, WAF 1, cdk4 inhibitor, MTS1, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatrophin, TGF-beta, TGF-α, VEGF, TNF-α, TNF-beta, cathepsin K, cytochrome P-450, farnesyltransferase, glutathione-S transferase, heparanase, HMG CoA synthetase, n-acetyltransferase, phenylalanine hydroxylase, phosphodiesterase, carboxy-terminal protease of ras, Notch, telomerase, TNF-converting enzyme, cadherin E, cadherin N, selectin, CD40, ANF, calcitonin, the corticotrophin-releasing factor, glucagon, gonadotropin, the gonadotropin-releasing hormone, growth hormone, the growth hormone-releasing factor, somatotropin, insulin, leptin, luteinizing hormone, luteinizing hormone-releasing hormone, PTH, thyroid hormones, thyroid-stimulating hormone, immunoglobulin CTLA4, hemagglutinin, MHC, VLA-4, the kallikrein-kininogen-kinin CD4 system, sis, hst, ras, abl, mos, myc, fos, jun, H-ras, ki-ras, c-fins, bcl-2, L-myc, c-myc, gip, gsp, HER-2, bombesin receptor, GABA receptor, EGFR, PDGFR, FGFR, NGFR, interleukin receptors, ion channel receptors, leukotriene receptor antagonists, lipoprotein receptors, opioid receptors, substance P receptors, retinoic acid and retinoid receptors, steroid receptors, T-cell receptors, thyroid hormone receptors, TNF receptors, tPA receptors, calcium pump, proton pump, Na/Ca exchanger, MRP 1, MRP2, P170, LRP, cMOAT, transferrin, APC, brca1, brca2, DCC, MCC, MTS1, NF1, NF2, nm23, p53, Rb, erythropoietin (EPO), leptins, adrenocorticotropin-releasing hormone (CRH), somatotropic hormone-releasing hormone (GHRH), gonadotropin-releasing hormone (GnRH), thyrotropin-releasing hormone (TRH), prolactin-releasing hormone (PRH), melatonin-releasing hormone (MRH), prolactin-inhibiting hormone (PIH), somatostatin, adrenocorticotropin hormone (ACTH), somatotropic hormone or growth hormone (GH), luteinizing hormone (LH), follicle-stimulating hormone (FSH), thyrotropin (TSH or thyroid-stimulating hormone), prolactin, oxytocin, antidiuretic hormone (ADH or vasopressin), melatonin, Müllerian inhibiting factor, calcitonin, parathyroid hormone, gastrin, cholecystokinin (CCK), Arg-vasopressin, thyroid hormones, azoxymethane, triiodothyronine, LIF, amphiregulin, soluble thrombomodulin, SCF, osteogenic protein 1, BMPs, MGF, MGSA, heregulins, melanotropin, secretin, insulin-like growth factor I (IGF-I), insulin-like growth factor II (IGF-II), atrial natriuretic peptide (ANP), human chorionic gonadotropin (hCG), insulin, glucagon, somatostatin, pancreatic polypeptide (PP), leptin, neuropeptide Y, renin, angiotensin I, angiotensin II, factor VIII, factor IX, tissue factor, factor VII, factor X, thrombin, factor V, factor XI, factor XIII, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15) interleukin 16 (IL-16), interleukin 24 (IL-24), tumor necrosis factor α (TNF-α), interferons alpha, beta, gamma, CD3, CD134, CD137, ICAM-1, LFA-1, LFA-3, chemokines including RANTES 1α, MIP-1α, MIP-1β, nerve growth factor (NGF), WT1 protein encoded by the Wilms' tumor suppressor gene, platelet-derived growth factor (PDGF), transforming growth factor beta (TGF-beta), bone morphogenetic proteins (BMPs), fibroblast growth factors (FGF and KGF), epidermal growth factor (EGF and related factors), vascular endothelial growth factor (VEGF), granulocyte colony-stimulating factor (GCSF), glial growth factor, keratinocyte growth factor, endothelial growth factor, glial-cell line-derived, neurotrophic factor (GDNF), alpha 1-antitrypsin, tumor necrosis factor, granulocyte-macrophage colony-stimulating factor (GM-CSF), cardiotrophin-1 (CT-1), oncostatin M (OSM), serpin (A1, A2, A3, A4, A5, A6, A7, A8, A9, A10, A11, A12, A13, B1, B2, B3, B4, B5, B6, B7, B8, B9, B10, B11, B12, B13, C1, D1, E1, E2, F1, F2, G1, H1, I1 and I2), cyclosporine, fibrinogen, the EDA domain of fibronectin, lactoferrin, tissue-type plasminogen activator (tPA), chymotrypsin, immunoglobins, hirudin, superoxide dismutase, imiglucerase, β-glucocerebrosidase, α-glucosidase, α-L-iduronidase, iduronate-2-sulfatase, galsulfase, human α-galactosidase A, α-1 proteinase inhibitor, lactase, pancreatic enzymes (lipase, amylase, protease), adenosine deaminase, immunoglobulins, albumin, botulinum toxins type A and B, collagenase, human deoxyribonuclease I, hyaluronidase, papain, L-asparaginase, lepirudin, streptokinase, porphobilinogen deaminase (PBGD), cell transforming factor beta (TGF-β) inhibitor peptides, IL10 inhibitors, FoxP3 inhibitors, TNFα inhibitors, VEGF inhibitors, PD-1 inhibitors and CD152 inhibitors.

The terms “prevent,” “preventing,” and “prevention”, as used herein, refer to inhibiting the inception or decreasing the occurrence of a disease in a subject. Prevention may be complete (e.g. the total absence of pathological cells in a subject) or partial. Prevention also refers to a reduced susceptibility to a clinical condition.

The term “prime-boost immunization strategy”, as used herein, refers to a vaccination method where the antigen is delivered twice to the patient: i) first by a priming vector ii) followed by a different second or boosting vector carrying the same antigen or parts thereof, called, respectively, priming vaccine and boosting vaccine.

The term “priming vaccine” as used herein refers to a vaccine comprising an agent(s) that encodes the target antigen to which an immune response is to be generated. Priming vaccines of the invention are administered to the subject or host in an amount effective to elicit an immune response to the target antigen.

The expression “promoter sequence”, as used herein, refers to a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease SI), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences. The terms “a sequence operably linked to a given promoter” or “under operative control”, are used herein indistinctly and refer to an arrangement of a sequence and a promoter region so that the sequence will be transcribed by the RNA polymerase into RNA. If the sequence is a coding sequence, it will then be translated into a polypeptide. The coding sequence and the promoter region need not be contiguous to one another so long as the transcribed sequence is ultimately processed to produce the desired protein.

The term “recombinant bacterium”, as used herein, refers to any bacterium that has been modified by the introduction of heterologous DNA. “Wild-type” or “control” bacterium include bacterium as found in its natural state without genetic manipulation or that are substantially identical to the recombinant bacterium, but do not express one or more of the proteins encoded by the heterologous DNA (e.g. contains a plasmid without the coding sequence of the heterologous polypeptide of interest). The term is intended to include progeny of the bacterium originally modified by the introduction of heterologous DNA.

The term “selection marker” or “selectable marker”, as used herein, relates to a nucleic acid sequence which upon intracellular expression is capable of conferring either a positive or negative selection marker or phenotypic characteristic for the cell expressing the sequence. This term includes both positive and negative selection markers. A “positive selection marker” is a nucleic acid sequence that allows the survival of cells containing the positive selection marker under growth conditions that kill or prevent growth of cells lacking the marker. An example of a positive selection marker is a nucleic acid sequence which promotes expression of the neomycin resistance gene. Cells not containing the neomycin resistance gene are selected against by application of G418, whereas cells expressing the neomycin resistance gene are not harmed by G418 (positive selection). A “negative selection marker” is a nucleic acid sequence that kills, prevents growth of or otherwise selects against cells containing the negative selection marker, usually upon application of an appropriate exogenous agent. An example of a negative selection marker is a nucleic acid sequence which promotes expression of the thymidine kinase gene of herpes simplex virus (HSV-TK). Cells expressing HSV-TK are selected against by application of ganciclovir (negative selection), whereas cells not expressing the gene are relatively unharmed by ganciclovir. In an embodiment, the polynucleotide according to the present invention comprises at least one selection marker, wherein said selection marker does not confer antibiotic sensitivity or antibiotic resistance to a cell carrying said polynucleotide.

The term “origin of replication” or “replication origin” or “ORI”, as used herein, relates to a particular genome sequence at which the replication is initiated. Bacteria typically comprise a single origin of replication per circular chromosome, while eukaryotes often have multiple origins of replication on each linear chromosome that initiate at different times. In a particular embodiment, the second polynucleotide of the invention comprises a mycobacterial origin of replication, preferably a mycobacterial oriM origin of replication. In a more particular embodiment, the second polynucleotide of the invention comprises a second origin of replication from a prokaryotic organism, preferably a oriE origin of replication from Escherichia coli.

The terms “shuttle plasmid” or “shuttle vector”, as used herein, refer to a plasmid DNA capable of replicating as a plasmid in bacteria and as a phage in mycobacteria. Shuttle plasmids capable of replicating in both bacteria and mycobacteria are known in the art. See O'Donnell M, et al., U.S. Pat. No. 5,591,632, O'Donnell M, et al., U.S. Pat. No. 5,776,465, and Bloom B, et al., U.S. Pat. No. 6,270,776).

The term “signal peptide” or “signal sequence”, as used herein, refers to a peptide serves to direct a protein containing such a sequence from the endoplasmic reticulum of a cell to the Golgi apparatus and ultimately to a lipid bilayer (e.g. for secretion). The term “subject”, as used herein, refers to an individual, plant or animal, such as a human beings, a non-human primate (e.g. chimpanzees and other apes and monkey species), a farm animal (e.g. birds, fish, cattle, sheep, pigs, goats and horses), a domestic mammal (e.g. dogs and cats), or a laboratory animal (e.g. rodents, such as mice, rats and guinea pigs. The term does not denote a particular age or sex. The term “subject” encompasses an embryo and a fetus.

The term “treat” or “treatment”, as used herein, refers to the administration of a pharmaceutical composition of the invention or of a medicament containing it to control the progression of a disease after its clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival, compared with the expected survival if treatment was not applied. Within the context of the present invention, the terms “treat” and “treatment” refer specifically to preventing or slowing the infection and destruction of healthy CD4+ T cells in a HIV-1 infected subject. It also refers to the prevention and slowing the onset of symptoms of the acquired immunodeficiency disease such as extreme low CD4+ T cell count and repeated infections by opportunistic pathogens such as Mycobacteria spp., Pneumocystis carinii, and Pneumocystis cryptococcus. Beneficial or desired clinical results include, but are not limited to, an increase in absolute naïve CD4+ T cell count (range 10-3520), an increase in the percentage of CD4+ T cell over total circulating immune cells (range 1-50%), or an increase in CD4+ T cell count as a percentage of normal CD4+ T cell count in an uninfected subject (range 1-161%). “Treatment” can also mean prolonging survival of the infected subject as compared to expected survival if the subject did not receive any HIV targeted treatment.

The term “unit dose”, as used herein, pertains to the inocula of the present invention, refers to physically discrete units suitable as unitary dosages for animals, each unit containing a predetermined quantity of active material calculated to produce the desired immunogenic effect in association with the required diluents (i.e. carrier or vehicle). The specifications for the novel unit dose of an inoculum of this invention are dictated by and are directly dependent on (a) the unique characteristics of the active material and the particular immunologic effect to be achieved, and (b) the limitations inherent in the art for compounding such active material for immunologic use in animals and human subjects, as disclosed in detail herein, these being features of the present invention.

The term “vaccine”, as used herein, refers to an immunogenic composition for in vivo administration to a host, which may be a primate, especially a human host, to confer protection against a disease, particularly a viral, bacterial or parasitic disease. It comprises at least one proteinaceous agent that induces the stimulation of the host immune system and prevents or attenuates subsequent unwanted pathology associated with the host reactions to subsequent exposures of the pathogen.

The term “vector”, as used herein, denotes a nucleic acid molecule, linear or circular, that comprises the genome encoding all the proteins forming a viral particle (except a part or the total of the integrase protein) operably linked to additional segments that provide for its autonomous replication in a host cell of interest. Preferably, the vector is an expression vector, which is defined as a vector, which in addition to the regions of the autonomous replication in a host cell, contains regions operably linked to the genome of the invention and which are capable of enhancing the expression of the products of the genome according to the invention.

The term “viral particle”, as used herein, refers to a whole viral particle and not to a protein subunit or peptide. Viral particles (also known as virions) consist of two or three parts: the genetic material of the virus made from either DNA or RNA; a protein coat that protects these genes; and, in some cases, an envelope of lipids that surrounds the protein coat when they are outside a cell. The shape of the viral particle ranges from simple helical and icosahedral forms to more complex structures, depending on the virus.

The term “weak mycobacterium promoter”, as used herein, refers to a promoter which, when coupled to a gene and introduced into a mycobacterial strain, allows expression of the protein at levels which are lower than that obtained with strong mycobacterial promoters.

### 2. Recombinant Polynucleotides

In a first aspect, the invention relates to a polynucleotide (first polynucleotide of the invention) comprising
(i) a sequence encoding a polypeptide of interest,
(ii) a first auxotrophy complementing gene which confers an auxotrophic host strain carrying said gene the capability of growing in a medium that lacks a first auxotrophic factor, and
(iii) a second auxotrophy complementing gene which confers an auxotrophic host strain carrying said gene the capability of growing in a medium that lacks a second auxotrophic factor.

In an embodiment of the invention, the polypeptide of interest comprises an immunogenic polypeptide. In another embodiment, the polypeptide of interest is under the operative control of a mycobacterial weak promoter.

In a particular preferred embodiment, the polynucleotide according to the invention does not comprise any nucleotide sequence conferring sensitivity to an antibiotic or any nucleotide sequence conferring resistance to an antibiotic.

The immunogenic polypeptide can be homologous or heterologous and includes, but is not limited to, a viral antigen, a bacterial antigen, a fungal antigen, a differentiation antigen, a tumor antigen, an embryonic antigen, an antigen of oncogenes and mutated tumor-suppressor genes, a unique tumor antigen resulting from chromosomal translocations or derivatives thereof.

Viral antigens which are capable of eliciting an immune response against the virus include HIV-1 antigens (e.g. tat, nef, gp120 or gp160, gp40, p24, gag, env, vif, vpr, vpu, rev), human herpes viruses (e.g. gH, gL gM gB gC gK gE or gD or derivatives thereof), or Immediate Early protein (e.g. ICP27, ICP47, ICP4, ICP36 from HSV1 or HSV2), cytomegalovirus, especially human (e.g. gB or derivatives thereof), Epstein-Barr virus (e.g. gp350 or derivatives thereof), varicella zoster virus (e.g. gp1, II, Ill and IE63), or from a hepatitis virus like hepatitis B virus (e.g. hepatitis B surface antigen or hepatitis core antigen), hepatitis C virus (e.g. core, E1, NS3 or NS5 antigens), from paramyxoviruses such as respiratory syncytial virus (e.g. F and G proteins or derivatives thereof), from parainfluenza virus, from rubella virus (e.g. proteins E1 and E2), measles virus, mumps virus, human papilloma viruses (e.g. HPV6, 11, 16, 18, eg LI, L2, E1, E2, E3, E4, E5, E6, E7), flaviviruses (e.g. yellow fever virus, dengue virus, tick-borne encephalitis virus, Japanese encephalitis virus) or influenza virus cells (e.g. HA, NP, NA, or M proteins, or combinations thereof), or rotavirus antigens (e.g. VP7sc and other rotaviral components). See Fields B, et al., Eds., “Fundamental Virology”, 2^{nd} Ed. (Raven Press, New York, N.Y., US, 1991).

Bacterial or protozoal antigens which can be encoded by the polynucleotide of the present invention include antigens from Neisseria spp, including N. gonorrhea and N. meningitidis (transferrin-binding proteins, lactoferrin binding proteins, PiIC and adhesins); antigens from S. pyogenes (e.g. M proteins or fragments thereof and C5A protease); antigens from S. agalactiae, S. mutans; H. ducreyi; Moraxella spp, including M. catarrhalis, also known as Branhamella catarrhalis (e.g. high and low molecular weight adhesins and invasins); antigens from Bordetella spp, including B. pertussis (e.g. parapertussis and B. bronchiseptica (e.g. pertactin, pertussis toxin or derivatives thereof, filamenteous hemagglutinin, adenylate cyclase, fimbriae); antigens from Mycobacterium spp, including M. tuberculosis, M. bovis, M. leprae, M. avium, M. paratuberculosis, M. smegmatis; Legionella spp, including L. pneumophila; (e.g. ESAT6, Antigen 85A, -B or -C, MPT 44, MPT59, MPT45, HSPIO, HSP65, HSP70, HSP 75, HSP90, PPD 19 kDa [Rv3763], PPD 38 kDa [Rv0934]); antigens from Escherichia spp, including enterotoxic E. coli (e.g. colonization factors, heat-labile toxin or derivatives thereof, heat-stable toxin or derivatives thereof), antigens from enterohemorragic E. coli and enteropathogenic E. coli (e.g. shiga toxin-like toxin or derivatives thereof); antigens from Vibrio spp, including V. cholera (e.g. cholera toxin or derivatives thereof); antigens from Shigella spp, including S. sonnei, S. dysenteriae, S. flexnerii; Yersinia spp, including Y. enterocolitica (e.g. a Yop protein); antigens from Y. pestis, Y. pseudotuberculosis; Campylobacter spp, including C. jejuni (e.g. toxins, adhesins and invasins); antigens from Salmonella spp, including S. typhi, S. paratyphi, S. choleraesuis, S. enteritidis; Listeria spp, including L. monocytogenes; Helicobacter spp, including H. pylori (e.g. urease, catalase, vacuolating toxin); antigens from Pseudomonas spp, including P. aeruginosa; Staphylococcus spp, including S. aureus, S. epidermidis; Enterococcus spp, including E. faecalis, E. faecium; Clostridium spp, including C. tetani (e.g. tetanus toxin and derivative thereof); antigens from C. botulinum (e.g. botulinum toxin and derivative thereof), antigens from C. difficile (e.g. clostridium toxins A or B and derivatives thereof); antigens from Bacillus spp, including B. anthracis (e.g. anthrax toxin and derivatives thereof); Corynebacterium spp, including C. diphtheriae (e.g. diphtheria toxin and derivatives thereof); antigens from Borrelia spp, including B. burgdorferi (e.g. OspA, OspC, DbpA, DbpB); antigens from B. garinii (e.g. OspA, OspC, DbpA, DbpB), B. afzelii (e.g. OspA, OspC, DbpA, DbpB), antigens from B. andersonfi (e.g. OspA, OspC, DbpA, DbpB), antigens from B. hermsii; Ehrlichia spp, including E. equi and the agent of the Human Granulocytic Ehrlichiosis; Rickettsia spp, including R. rickettsii; Chlamydia spp, including C. trachomatis (e.g. MOMP, heparin-binding proteins); antigens from Chlamydia pneumoniae (e.g. MOMP, heparin-binding proteins), antigens from C. psittaci; Leptospira spp, including L. interrogans; Treponema spp, including T. pallidum (e.g. the rare outer membrane proteins), antigens from T. denticola, T. hyodysenteriae; antigens from Plasmodium spp, including P. falciparum; Toxoplasma spp and T. gondii (e.g. SAG2, SAGS, Tg34); antigens from Entamoeba spp, including E. histolytica; Babesia spp, including B. microti; Trypanosoma spp, including T. cruzi; Giardia spp, including G. lamblia; Leishmania spp, including L. major; Pneumocystis spp, including P. carinii; Trichomonas spp, including T. vaginalis; Schisostoma spp, including S. mansoni, or derived from yeast such as Candida spp, including C. albicans; Cryptococcus spp, including C. neoformans; antigens from M. tuberculosis (e.g. Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16kDal., Tb Ra12, Tb H9, Tb Ra35, Tb38-1, Erd 14, DPV, MTI, MSL, mTIC2 and hTCC1); antigens from Chlamydia spp (e.g. the High Molecular Weight Protein (HWMP), ORF3 (EP 366 412)), and putative membrane proteins (Pmps); antigens from Streptococcus spp, including S. pneumoniae (PsaA, PspA, streptolysine, choline-binding proteins, the protein antigen Pneumolysin, and mutant detoxified derivatives thereof); antigens derived from Haemophilus spp, including H. influenzae type B (e.g. PRP and conjugates thereof); antigens from non typeable H. influenzae (e.g. OMP26, high molecular weight adhesins, P5, P6, protein D and lipoprotein D, and fimbrin and fimbrin derived peptides, or multiple copy variants or fusion proteins thereof); antigens derived from Plasmodium falciparum (e.g. RTS.S, TRAP, MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp). Preferably, the antigen is MSP1 of Plasmodium falciparum or Ag 85-B of Mycobacterium spp.

Fungal antigens which can be encoded by the polynucleotides of the present invention include, without limitation, candida fungal antigen components; histoplasma fungal antigens, such as heat shock protein 60 (HSP60) and other histoplasma fungal antigen components; cryptococcal fungal antigens, like capsular polysaccharides and other cryptococcal fungal antigen components; coccidiodes fungal antigens, such as spherule antigens, and other coccidiodes fungal antigen components; and tinea fungal antigens, like trichophytin and other coccidiodes fungal antigen components.

Allergens or environmental antigens which can be encoded by the polynucleotides of the present invention include, without limitation, antigens derived from naturally occurring allergens, such as pollen allergens (e.g. tree-, herb, weed- and grass pollen allergens), insect allergens (inhalant, saliva and venom allergens), animal hair and dandruff allergens, and food allergens. Important pollen allergens from trees, grasses and herbs originate from the taxonomic orders of fagales, oleales, pinoles and platanaceae including Louisiana birch (Betula), alder (Alnus), hazel (Corylus), hornbeam (Carpinus) and olive (Olea), cedar (Cryptomeria and Juniperus), plane tree (Platanus), the order of poales (e.g. grasses of the genera Lolium, Phleum, Poa, Cynodon, Dactylis, Holcus, Phalaris, Secale and Sorghum) the orders of Asterales and Urticales (e.g. herbs of the genera Ambrosia, Artemisia and Parietaria). Other allergen antigens that may be used include allergens from house dust mites of the genus Dermatophagoides and Euroglyphus, storage mite (e.g. Lepidoglyphys, Glycyphagus and Tyrophagus), those from cockroaches, midges and fleas (e.g. Blatella, Periplaneta, Chironomus and Ctenocepphalides), those from mammals (e.g. cat, dog and horse), birds, venom allergens including such originating from stinging or biting insects such as those from the taxonomic order of Hymenoptera including bees (e.g. superfamily Apidae), wasps and ants (e.g. superfamily Formicoidae). Still other allergen antigens that may be used include inhalation allergens from fungi (e.g. from the genera Alternaria and Cladosporium).

Tumor antigens which can be encoded by the polynucleotides of the present invention include, without limitation, MAGE, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its antigenic epitopes CAP-1 and CAP-2, etv6, aml1, Prostate Specific Antigen (PSA) and its antigenic epitopes PSA-1, PSA-2, and PSA-3, prostate-specific membrane antigen (PSMA), T-cell receptor/CD3-ζ chain, MAGE-family of tumor antigens (e.g. MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g. GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p2lras, RCAS1, α-fetoprotein, E-cadherin, α-catenin, 13-catenin, γ-catenin, pl2Octn, gp100Pme1117, PRAME, NY-ESO-1, cdc27, adenomatous polyposis coli protein (APC), fodrin, Connexin 37, Ig-idiotype, p15, gp75, GM2 and GD2 gangliosides, viral products, like human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, and c-erbB-2, acute lymphoblastic leukemia (etv6, amll, cyclophilin b), B cell lymphoma (Ig-idiotype), glioma (E-cadherin, a-catenin, 13-catenin, 7-catenin, p120ctn), bladder cancer (p2lras), biliary cancer (p2lras), breast cancer (MUC family, HER2/neu, c-erbB-2), cervical carcinoma (p53, p2lras), colon carcinoma (p2lras, HER2/neu, c-erbB-2, MUC family), colorectal cancer (Colorectal associated antigen (CRC)-0017-1A/GA733, APC), choriocarcinoma (CEA), epithelial cell cancer (cyclophilin b), gastric cancer (HER2/neu, c-erbB-2, ga733 glycoprotein), hepatocellular cancer, Hodgkins lymphoma (lmp-1, EBNA-1), lung cancer (CEA, MAGE-3, NY-ESO-1), lymphoid cell-derived leukemia (cyclophilin b), melanoma (p15 protein, gp75, oncofetal antigen, GM2 and GD2 gangliosides, Melan-A/MART-1, cdc27, MAGE-3, p2lras, gp100Pme1117), myeloma (MUC family, p2lras), non-small cell lung carcinoma (HER2/neu, c-erbB-2), nasopharyngeal cancer (lmp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus (viral products e.g. human papilloma virus proteins), testicular cancer (NY-ESO-1), and T cell leukemia (HTLV-1 epitopes).

In a preferred embodiment, the immunogenic polypeptide comprises an HIV polypeptide, a Mycobacteria spp polypeptide, a Plasmodium falciparum epitope, or an immunologically active epitope thereof. In preferred embodiments, the antigenic polypeptide is not a sequence encoding the mycobacterium α-antigen or the Schistosoma mansoni glutathione-S-transferase (GST).

Any HIV antigen that can induce an immune response in the host is useful for the present invention. Examples include HIV-I gp120; HIV-I gp41; HIV-I gp160; HIV-I pol; HIV-I nef; HIV-I tat; HIV-I rev; HIV-I vif; HIV-I vpr; EIV-I vpu; HIV-I gag; HIV-2 gp120; HIV-2 gp160; HIV-2 gp41; HIV-2 gag; HIV-2 pol; HIV-2 nef; HIV-2 tat; HIV-2 rev; HIV-2 vif; HIV-2-vpr; HIV-2-vpu; and HIV-2-vpx. In a preferred embodiment, the HIV-1 polypeptide is gp120). In another preferred embodiment, the HIV-1 antigenic polypeptide is HIVA. See Table 1.

In a preferred embodiment, the immunogenic polypeptide comprises a Mycobacteria spp polypeptide. Any mycobacterium polypeptide that can induce an immune response in the host is useful for the present invention. Examples of mycobacterium antigens include, without being limited to, CFP-10, ESAT-6, Rv0288, Rv0287, Rv3019c, HSP65, HSP70, Acr1, Acr2, ag85A, Ag85B, MPB83, MPB70, 19 kDa antigen, 38 kDa antigen (Pst-1), Rv0909, Rv3616c, Mb2555, Mb2890, Mb3895, Rv3879c, Rv1196 and Rv1769.

In a preferred embodiment, the immunogenic polypeptide comprises a plasmodium polypeptide. Any Plasmodium falciparum epitope that can induce an immune response in the host is useful for the present invention. Non-limitative examples include fragments MSP1-33 and MSP1-42 of the merozoite surface protein, epitopes from the P. falcifarum erythrocyte membrane protein 1 (PfEMP1) and p190 precursor protein.

In a particular embodiment, the sequence encoding the polypeptide of interest is fused in frame to a sequence coding a signal sequence active in mycobacteria, thereby allowing the secretion of the antigenic polypeptide across the mycobacterial membrane and increasing the immune response towards the antigen in the host organism.

In a preferred embodiment, the signal peptide or signal sequence contains about 20 amino acids which occurs at the N-terminus of secretory and integral membrane proteins and which contains a large number of hydrophobic amino acid residues. A signal sequence may contain at least about 14-28 amino acid residues, preferably about 16-26 amino acid residues, more preferably about 18-24 amino acid residues, and more preferably about 20-22 amino acid residues, and has at least about 40-70 percent, preferably about 50-65 percent, and more preferably about 55-60 percent hydrophobic amino acid residues (e.g. alanine, valine, leucine, isoleucine, phenylalanine, tyrosine, tryptophan, or proline). The structural characteristics of the signal peptide lie in that the signal peptide contains Met, corresponding to the initiation codon ATG at the NH2-terminal. Two to three basic amino acids (e.g. lys, arg) are present around the NH2-terminal. Fifteen to twenty-five hydrophobic amino acids follow the basic amino acids, and a cleavage site of the signal peptide is composed of amino acid residues having a short side chain (e.g. ala, gly). These structural characteristics are common to various species of organism but their amino acid sequences and nucleotide sequences of signal peptide are subtly different. Therefore, it is desirable to use a signal sequence of a protein actually extracellularly secreted from mycobacteria in large quantities as it is, or to use synthetic DNA obtained by artificially synthesizing such a signal sequence. Suitable signal sequences for use in the polynucleotides of the present invention include, without limitation, the signal sequence of the a antigen of M. bovis BCG, the signal sequence of the MPB64 protein, the signal sequence of the MPB70, the signal sequence of the a antigen of Mycobacterium kansasii. In a preferred embodiment, the signal sequence corresponds to the signal sequence of Mycobacterium tuberculosis 19 KDa lipoprotein or a functionally equivalent variant thereof. The signal sequence of Mycobacterium tuberculosis 19 KDa lipoprotein is shown in SEQ ID NO:026:

| | | |
|---|---|---|
| 1 | MKRGLTVAVA GAAILVAGLS G | 21 |

which is encoded by nucleotides 198 to 261 of the polynucleotide shown in the NCBI database under accession number X07945.

The expression “functionally equivalent variant”, when referred to a signal sequence relates to any peptide sequence which derives from the signal sequence of one of the sequences mentioned above by the addition, deletion or substitution of one or more amino acids while still preserving the ability to direct a passenger protein across the secretory pathway. A suitable assay for determining whether a given peptide is an adequate signal peptide for secretion in mycobacteria is described in the art. See Yamada T, et al., U.S. Pat. No. 6,015,696. This assay involves the use of strains carrying a gene construct wherein a reporter peptide is cloned as a fusion with the putative signal peptide. After culture of the strain, the detection of a substantial amount of said reporter peptide in the culture supernatant is indicative that the putative signal peptide is indeed capable of promoting the secretion of the reporter peptide. Detection of the reporter peptide in the culture supernatant is usually carried out by concentration of the culture supernatant followed by conventional protein detection technology (e.g. western blot, ELISA).

Signal sequence variants according to the present invention include amino acid sequences that are at least 60%, 70%, 80%, 90%, 95% or 96% similar or identical to known signal sequences as defined herein above. As known in the art the “similarity” between two proteins is determined by comparing the amino acid sequence and its conserved amino acid substitutes of one protein to a sequence of a second protein. The degree of identity between two proteins is determined using computer algorithms and methods that are widely known for the persons skilled in the art. The identity between two amino acid sequences is preferably determined by using the BLASTP algorithm. See Altschul, 1977, 1990, supra.

In a preferred embodiment, the polypeptide of interest further comprises an endosomalytic polypeptide. Endosomalytic polypeptides according to the invention include, without being limited to, Listeria monocitogenes listeriolysin (GenBank accession nos. CAA59919 or CAA42639), Escherichia coli hemolysin (GenBank accession nos. AAC24352 or CAA0535), Clostridium perfringens perfringolysin (GenBank accession nos. P19995 or AAA23271) and the Mycobacterium tuberculosis phospholipase C protein family.

Additionally, the polynucleotide of the invention may further comprise a sequence encoding a detection/purification tag (i.e. a sequence encoding a peptide of known sequence and not present in native host cells that allows the detection or the purification of the antigenic peptide). Adequate detection/purification tags includes hexa-his (metal chelate moiety), hexa-hat GST (glutathione S-tranferase) glutathione affinity, calmodulin-binding peptide (CBP), strep-tag, cellulose binding domain, maltose binding protein, S-peptide tag, chitin binding tag, immuno-reactive epitopes, epitope tags, E2tag, HA epitope tag, Myc epitope, FLAG epitope, AU1 and AU5 epitopes, GIu-GIu epitope, KT3 epitope, IRS epitope, Btag epitope, protein kinase-C epitope, VSV epitope or any other tag as long as the tag does not affect the stability of the protein or the immunogenicity of the antigen attached thereto.

The promoter used in the nucleic acid sequences of the present invention is a heterologous promoter. In particular, the promoter used in the nucleic acid sequences of the present invention is a heterologous weak mycobacterium promoter.

The strong or weak promoter activity is associated with the level of protein expression regulated by such promoter. The promoter activity, the host strain harboring the plasmid DNA and the toxicity of the heterologous protein expressed could affect the plasmid DNA stability and the occurrence of undesirable genetic rearrangements. An example of a method for assessing promoter activity is by utilizing a reporter gene system (GFP, E. coli β-galactosidase, β lactamase). According to this approach, the promoter could be considered a weak mycobacterium promoter if the expression levels of a reporter gene operatively linked to said weak promoter is lower than the expression level of the same reporter gene when the reporter gene is under the operative control of a strong mycobacterial promoter, such as the M. bovis T101 promoter, the M. bovis hsp60 promoter, or the S16 M. smegmatis promoter. See Bashyam M, et al., J. Bacteriol. 1996; 178:4847-4853, Thole J, et al., Infect. Immun. 1987; 55:1466-1475, Stover C, et al., Nature 1991; 351:456-460, and Dellagostin O, et al., Microbiol. 1995; 141:1785-1792. A promoter can be considered as weak if, when a gene construct comprising the sequence encoding HIV-1 gp120 under the control of said promoter is expressed in M. bovis BCG, the construct remains stable over an extended period of time. Conversely, when a gene construct comprising the sequence encoding HIV-1 gp120 under the control of said promoter is expressed in M. bovis BCG and the gene suffers deletions over time, this is indicative that the promoter is a strong promoter. The term “reduced level of promoter activity”, as used herein, refers to promoters showing a transcriptional activity which is at least 90%, 80%, 70%, 60%, 50%, 40%, 30%, 20%, 10%, 5%, 4%, 3%, 2%, 1%, 0.5% or less of the activity of a reference strong promoter.

Suitable promoters for use in the present invention include, without limitation, the T125 promoter as described in Das Gupta S, et al., J. Bacteriol. 1993; 175:5186 and having the sequence SEQ ID NO:027:

| | |
|---|---|
| 1 | CCGAGGTAAG GACTGAGCAT GGGCCCGATA AAGTGACTAT TATGGATTTC |

or the Mycobacteria spp α-antigen promoter described in Kremer L, et al., J. Bacteriol. 1995; 177:642-653 having the sequence SEQ ID NO:028:

| | |
|---|---|
| 1 | TCTAGAATAC GGAAATGAGA CGACTTTGCG CCCGAATCGA CATTTGGCCT CCACACACGG |
| 61 | TATGTTCTGG CCCGAGCACA CGACGACATA CAGGACAAAG GGGCACAGGT ATGCATATG |

In the particular case that the heterologous promoter is the α-antigen promoter, then the polynucleotide which is operatively coupled to the promoter is not the gene encoding the α-antigen.

The invention also contemplates the use of promoters which are functionally equivalent variants of the specific promoters mentioned above. The term “functionally equivalent variant”, when referring to a promoter, refers to a promoter which results from the insertion, deletion or substitution of one or more nucleotides but wherein the transcriptional promoting activity is substantially preserved as determined using any of the assays mentioned above. “Functionally equivalent variants of the promoters” used herein comprise promoters showing a substantial homology with the α-antigen promoter or the T125 promoter, wherein a substantial homology between two nucleic acid sequences is considered when both nucleic acid sequences show at least 60%, advantageously of at least 70%, preferably of at least 85%, and more preferably of at least 95% of sequence identity over their entire length. The degree of identity between two polynucleotides is determined using computer algorithms and methods that are widely known for the persons skilled in the art. The identity between two amino acid sequences is preferably determined by using the BLASTN algorithm. See Altschul S, et al., Nuc. Acids Res. 1977; 25:3389-3402 and Altschul S, et al., J. Mol. Biol. 1990; 215:403-410.

Suitable weak mycobacterium promoters according to the present invention are promoters active in several Mycobacterium spp (e.g. M. smegmatis and M. bovis BCG), but not in E. coli. See Kremer L, et al., J. Bacteriol. 1995; 177(3):642-653. Another example of mycobacterial promoters according to the present invention, are promoters controlling the expression of heat shock proteins. In a particular embodiment, the weak mycobacterium promoter is the Mycobacteria spp α-antigen promoter or a functionally equivalent variant thereof.

The polynucleotide of the invention comprises a first auxotrophy complementing gene which confers an auxotrophic host strain carrying said gene the capability of growing in a medium that lacks a first auxotrophic factor. In a particular embodiment, the first auxotrophy complementing gene is a lysine auxotrophy complementing gene. In a preferred embodiment, the first auxotrophy complementing gene is a gene capable of complementing a Mycobacterium lysine auxotrophy (i.e. a gene capable of complementing a mycobacterial strain defective in the synthesis of lysine). In a more preferred embodiment, the first auxotrophy complementing gene is the lysA gene.

The polynucleotide of the invention comprises a second auxotrophy complementing gene which confers an auxotrophic host strain carrying said gene the capability of growing in a medium that lacks a second auxotrophic factor. In a particular embodiment, the second auxotrophy complementing gene is a glycine auxotrophy complementing gene. In a preferred embodiment, the second auxotrophy gene is an E. coli gene capable of complementing an E. coli glycine auxotrophy ((i.e. a gene capable of complementing an E. coli defective in the synthesis of glycine). In a more preferred embodiment, the second auxotrophy complementing gene is the glyA gene.

In another embodiment, the first polynucleotide of the invention comprises a mycobacterial origin of replication. In a preferred embodiment, the mycobacterial origin of replication is oriM.

In another embodiment, the first polynucleotide of the invention comprises a second origin of replication from a prokaryotic organism. In a more preferred embodiment, the second origin of replication is oriE.

In a preferred embodiment, the first polynucleotide of the invention comprises a first mycobacterial origin of replication (preferably OriM) and a second origin of replication from a prokaryotic organism. In a more preferred embodiment, preferably oriE.

In a further aspect, the invention relates to a polynucleotide (second polynucleotide of the invention) comprising

(i) a sequence encoding a polypeptide of interest,

(ii) a mycobacterial origin of replication, and

(iii) at least one selection marker,

wherein said polynucleotide does not comprise any nucleotide sequence conferring antibiotic sensitivity or antibiotic resistance to a cell carrying said polynucleotide.

Polypeptides of interest according to the invention have been previously described in the context of the first polynucleotide of the invention. In a particular embodiment, the polypeptide of interest comprises an immunogenic polypeptide and an endosomalytic polypeptide.

Immunogenic peptides according to the invention have been previously described. In a preferred embodiment, the immunogenic polypeptide comprises an HIV polypeptide, a Mycobacteria spp polypeptide, a Plasmodium falciparum epitope, or an immunologically active epitope thereof. In preferred embodiment, the antigenic polypeptide is not a sequence encoding the mycobacterium α-antigen or the Schistosoma mansoni glutathione-S-transferase (GST). In a more particular embodiment, the HIV polypeptide is selected from gp120, HIVA or HIV-c. In a more particular embodiment, the Mycobacteria spp polypeptide is Ag85B from Mycobacterium bovis. In a more particular embodiment, the Plasmodium falciparum epitope is circumsporozoite surface protein (CSP) from Plasmodium berghei.

Endosomalytic peptides according to the invention have been previously described. Preferred endosomalytic polypeptide comprise Listeria monocitogenes listeriolysin, Clostridium perfringens perfingolysin, Mycobacterium tuberculosis phospholipase C or a variant thereof.

In a particular embodiment, the sequence encoding the polypeptide of interest is fused in frame to a sequence coding a signal sequence active in mycobacteria, thereby allowing the secretion of the antigenic polypeptide across the mycobacterial membrane and increasing the immune response towards the antigen in the host organism. Signal peptides or signal sequences have been previously described in the context of the first polynucleotide of the invention. In a particular embodiment, the sequence encoding the polypeptide of interest is fused in frame to a sequence encoding a signal sequence active in mycobacteria, preferably wherein the signal sequence corresponds to the signal sequence of Mycobacterium tuberculosis 19 KDa lipoprotein or a functionally equivalent variant thereof.

In a particular embodiment, the sequence encoding the polypeptide of interest is under the operative control of a heterologous weak mycobacterium promoter, preferably wherein the weak mycobacterium promoter is the Mycobacteria spp α-antigen promoter or a functionally equivalent variant thereof.

The second polynucleotide of the invention comprises a mycobacterial origin of replication. In a particular embodiment, the mycobacterial origin of replication is oriM. In a more particular embodiment, the second polynucleotide of the invention further comprises a second origin of replication from a prokaryotic organism, more particularly a oriE origin of replication.

Additionally, the second polynucleotide of the invention may further comprise a sequence encoding a detection/purification tag, as have been described previously in relation to the first polynucleotide of the invention. The second polynucleotide of the invention may comprise as well a promoter, as described previously.

The second polynucleotide of the invention comprises as well at least one selection marker, wherein said polynucleotide does not comprise any nucleotide sequence conferring antibiotic sensitivity or antibiotic resistance to a cell carrying said polynucleotide.

Selection markers according to the invention comprise the following:
sucrose hydrolase (cscA): sucrose hydrolase is an enzyme from Escherichia coli O157:H7 strain Sakai which is involved in sucrose utilisation. Transforming Escherichia coli K12 strains with sucrose hydrolase allows the cells to grow with sucrose as a sole carbon source, whereas the untransformed K12 strain cannot do,
nitroreductase (nfsI): nitroreductase is an Enterobacter cloacae enzyme which reduces nitrogen containing compounds,
haloalkane dehalogenase (dhlA): DhlA is haloalkane dehalogenase from Xanthobacter autotrophicus GJ10. It converts 1,2-dichloroethane (DCE) into the more toxic 2-chloroethanol and is successfully used as a counterselectable marker in plants, or
levansucrase (sacB): it is the levansucrase enzyme from Bacillus subtilis which converts sucrose into fructose polymers which are lethal to Esherichia coli (French C & Kowal M 2010; http://partsregistry.org/Part:BBa_K322921).

In a particular embodiment, the at least one selection marker is a first auxotrophy complementing gene which confers an auxotrophic host strain carrying said gene the capability of growing in a medium that lacks a first auxotrophic factor. In a more particular embodiment, the polynucleotide further comprises a second auxotrophy complementing gene which confers an auxotrophic host strain carrying said gene the capability of growing in a medium that lacks a second auxotrophic factor. In a particular preferred embodiment, the first auxotrophy gene is a gene capable of complementing a mycobacterium lysine auxotrophy or the second auxotrophy gene is an E. coli gene capable of complementing E. coli glycine auxotrophy, more preferably the gene capable of complementing a mycobacterium lysine auxotrophy is the lysA gene or the gene capable of complementing an E. coli glycine auxotrophy is the glyA gene.

### 3. Vectors

The first and/or second polynucleotides according to the invention may be conveyed in a vector in order to allow adequate propagation of the nucleic acid. Thus, in another aspect, the invention relates to a vector that comprises a polynucleotide according to the present invention.

A person skilled in the art will understand that there is no limitation as regards the type of vector which can be used because said vector can be a cloning vector suitable for propagation and for obtaining the polynucleotides or suitable gene constructs or expression vectors in different heterologous organisms suitable for purifying the conjugates. Thus, suitable vectors according to the present invention include prokaryotic expression vectors (e.g. pUC18, pUC19, Bluescript and their derivatives), mp18, mp19, pBR322, pMB9, CoIE1, pCR1, RP4, phages and shuttle vectors (e.g. pSA3 and pAT28), yeast expression vectors (e.g. vectors of the type of 2 micron plasmids), integration plasmids, YEP vectors, centromeric plasmids and the like, insect cell expression vectors (e.g. the pAC series and pVL series vectors), plant expression vectors, such as vectors of expression in plants (e.g. pIBI, pEarleyGate, pAVA, pCAMBIA, pGSA, pGWB, pMDC, pMY, pORE series vectors), and eukaryotic expression vectors based on viral vectors (e.g. adenoviruses, viruses associated to adenoviruses as well as retroviruses and lentiviruses), as well as non-viral vectors (e.g. pSilencer 4.1-CMV (Ambion®, Life Technologies Corp., Carlsbad, Calif., US), pcDNA3, pcDNA3.1/hyg pHCMV/Zeo, pCR3.1, pEFl/His, pIND/GS, pRc/HCMV2, pSV40/Zeo2, pTRACER-HCMV, pUB6N5-His, pVAX1, pZeoSV2, pCI, pSVL and pKSV-10, pBPV-1, pML2d and pTDT1).

Vectors may further contain one or more selectable marker sequences suitable for use in the identification of cells which have or have not been transformed or transfected with the vector. Markers include, for example, genes encoding proteins which increase or decrease either resistance or sensitivity to antibiotics or other compounds (e.g. hyg encoding hygromycin resistance (GenBank accession no. AF025746; or AF025747) and Kan^{R} (GenBank accession no. U75323), genes which encode enzymes whose activities are detectable by standard assays known in the art (e.g. β-galactosidase or luciferase), and genes which visibly affect the phenotype of transformed or transfected cells, hosts, colonies or plaques such as various fluorescent proteins (e.g. green fluorescent protein, GFP). Alternatively, the vectors of the present invention may carry a non-antibiotic selection marker, since it is not always ideal to use antibiotic resistance markers for selection and maintenance of plasmids in mycobacteria that are designed for use in humans and veterinary pharmaceutics including, for instance, genes encoding a catabolic enzyme which enables the growth of mycobacteria in medium containing a substrate of said catabolic enzyme as a carbon source. An example of such a catabolic enzyme includes, but is not restricted to, lacYZ encoding lactose uptake and beta-galactosidase (GenBank accession nos. J01636, J01637, K01483, or K01793). Other selection markers that provide a metabolic advantage in defined media include, but are not restricted to, galTK (GenBank accession no. X02306) for galactose utilization, sacPA (GenBank accession no. J03006) for sucrose utilization, trePAR (GenBank accession no. Z54245) for trehalose utilization and xylAB (GenBank accession nos. CAB 13644 and AAB41094) for xylose utilization. Alternatively, the selection can involve the use of antisense mRNA to inhibit a toxic allele, for instance the sacB allele (GenBank accession no. NP 391325), which renders Mycobacterium strains sensitive to sucrose.

In a preferred embodiment, the vector is an E. coli/mycobacterial shuttle vector. The invention contemplates the use of E. coli/mycobacterial shuttle extrachromosomal vectors as well as E. coli/mycobacterial shuttle integrative vectors. Both types of recombinant vectors can be used to introduce DNA of interest stably into mycobacteria, in which the DNA can then be expressed. In the case of an integrative shuttle plasmid, stable integration into the mycobacterial chromosomal or genomic DNA occurs via site specific integration. The DNA of interest is replicated as part of the chromosomal DNA. In the case of an extrachromosomal bacterial-mycobacterial shuttle plasmid, the DNA of interest is stably maintained extrachromosomally as plasmid DNA. Replication of the plasmid DNA occurs extrachromosomally as a plasmid (e.g. episomally). More than one copy of the episomal plasmid DNA can be found in the bacterial cytoplasm (i.e. multi-copy). For example, a gene or genes of interest is/are cloned into a bacterial-mycobacterial plasmid and introduced into a cultivable mycobacterium, where it undergoes episomal replication (extrachromosomal replication). Suitable shuttle vectors for use in the present invention include, without limitation, the pMV261, pMV361, pJH222 and pJH223 vectors. See Stover C, et al., Nature 1991; 351:456-460, Cayabyab C, et al., J. Viriol. 2006; 2:1645-1652, Lee M, et al., Proc. Natl. Acad. Sci. USA 1991; 88:3111-3115, Hopkins R, et al., Eur. J. Immunol. 2011; 41(12):3542-3552, Hopkins R, et al., PLoS ONE 2011; 6(5):e20067, Saubi N, et al., Clin. Develop. Immunol. 2011; doi:10.1155/2011/516219, Joseph J, et al., J. Biomed. Biotechnol. 2010; 2010:357370, and Rosario M, et al., J. Virol. 2010; 84(15):7815-7821.

Vectors according to the invention can also comprise at least one heterologous sequence encoding a cytokine, which is used to elicit augmented host responses to the immunogen Mycobacterium vector. The particular cytokine encoded by the Mycobacterium vector is not critical to the present invention includes, but not limited to, interleukin-4 (herein referred to as “IL-4”), IL-5, IL-6, IL-10, IL-12 p40, IL-12 p70, TGFβ and TNF□.

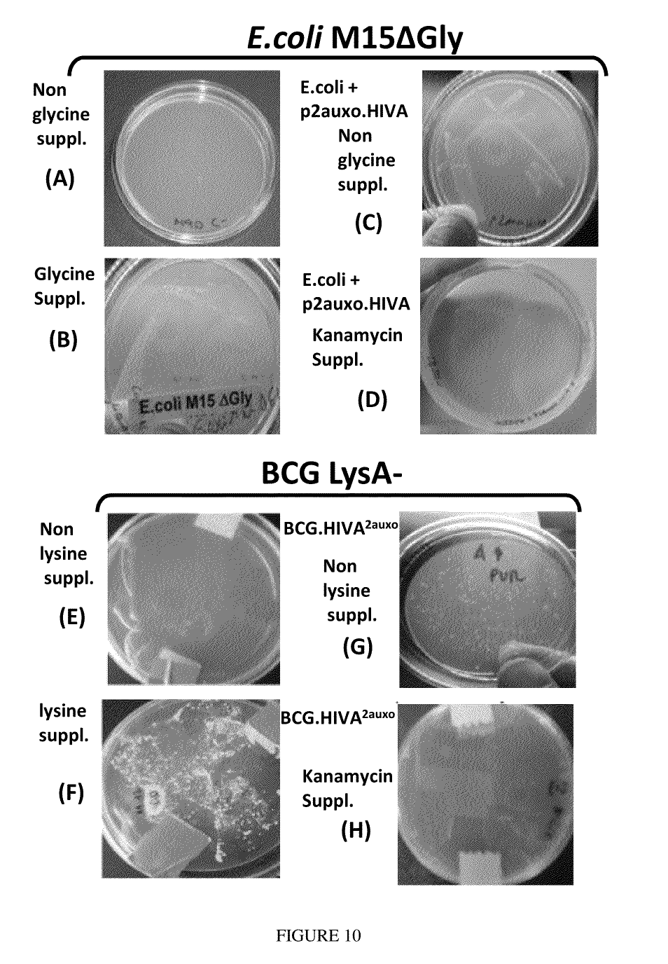

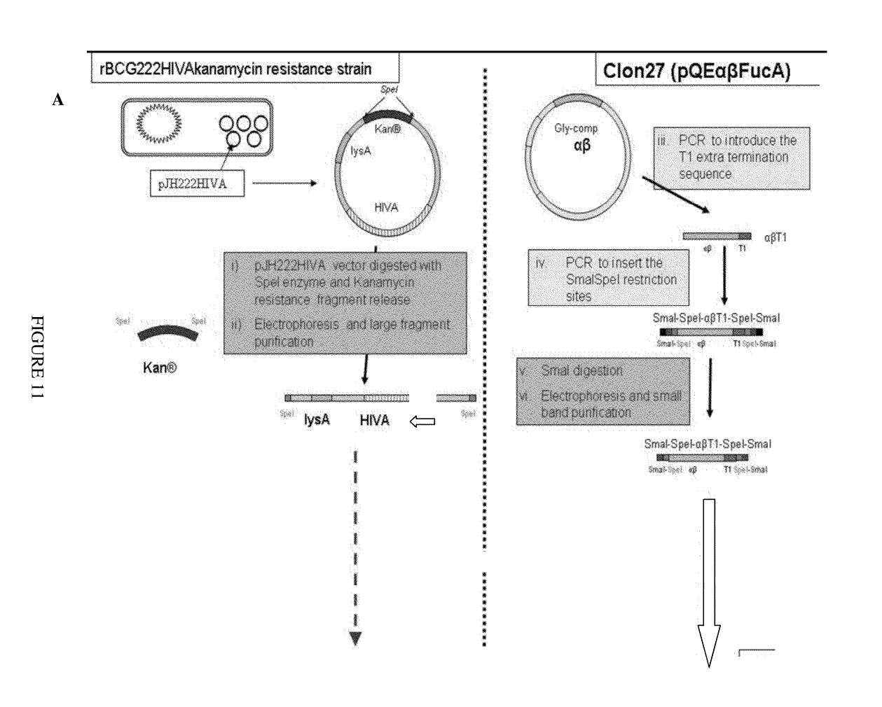

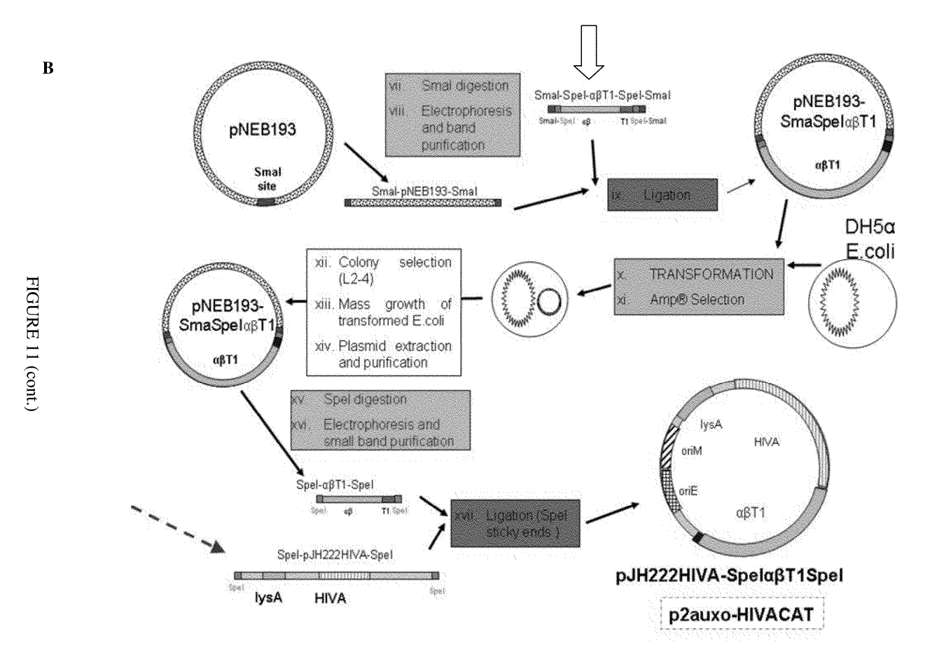

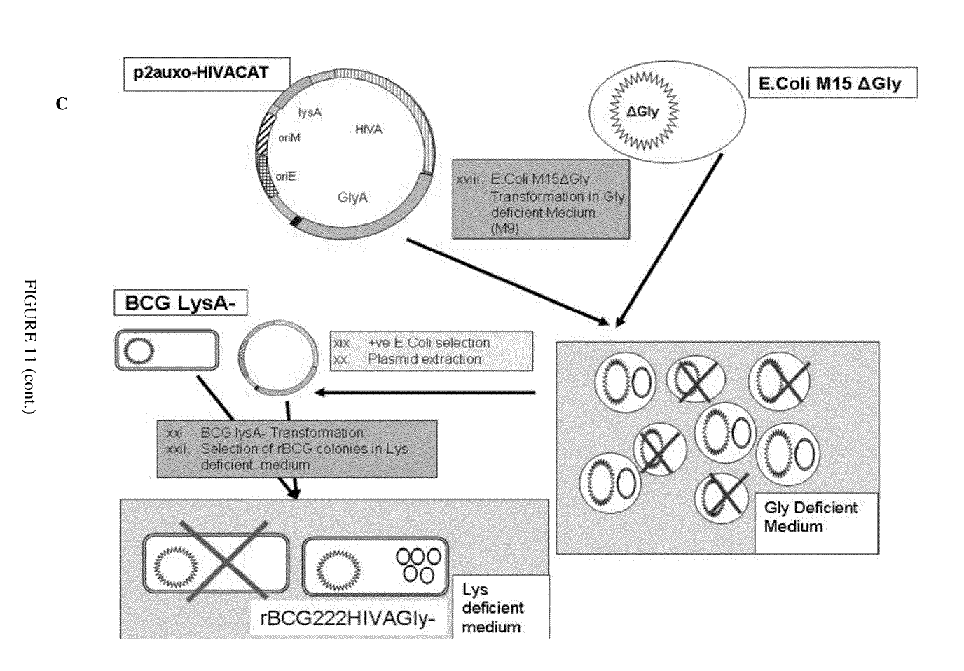

In a preferred embodiment of the invention, the vector is the plasmid p2auxo.HIVA (DSMZ accession no. DSM 26305). See Example 2 and FIG. 10.

### 4. Host Cells

The polynucleotides and vectors according to the invention may be provided within host cells. Thus, in another aspect, the invention relates to a recombinant bacterium cell characterized by having a polynucleotide or vector according to the invention.

According to the invention, the bacterium can be a gram negative bacterial cell, this term intended to include all facultative anaerobic Gram-negative cells of the family Enterobacteriaceace such as Escherichia, Shigella, Citrobacter, Salmonella, Klebsiella, Enterobacter, Erwinia, Kluyvera, Serratia, Cedecea, Morganella, Hafhia, Edwardsiella, Providencia, Proteus and Yersinia. In another preferred embodiment, the bacterium is a gram positive bacterial cell of the genus Mycobacteriaceae such as M. bovis-BCG, M. leprae, M. marinum, M. smegmatis and M. tuberculosis.

In a preferred embodiment of the invention, the recombinant bacterium cell is a recombinant Mycobacteria spp strain cell. The Mycobacteria spp strain includes, but is not limited to, M. tuberculosis strain CDC 1551 (i.e. Griffith D, et al., Am. J. Respir. Crit. Care Med. 1995; 152(2):808), M. tuberculosis strain Beijing (i.e. van Soolingen D, et al., J. Clinic. Microbiol. 1995; 33(12):3234-3238), M. tuberculosis strain H37Ra (ATCC accession no. 25177), M. tuberculosis strain H37Rv (ATCC accession no. 25618), M. bovis (ATCC accession nos. 19211 and 27291), M. fortuitum (ATCC accession no. 15073), M. smegmatis (ATCC accession nos. 12051 and 12549), M. intracellular (ATCC accession nos. 35772 and 13209), M. kansasii (ATCC accession nos. 21982 and 35775) M. avium (ATCC accession nos. 19421 and 25291), M. gallinarum (ATCC accession no. 19711), M. vaccae (ATCC accession nos. 15483 and 23024), M. leprae (ATCC accession no. 4233), M. marinarum (ATCC accession nos. 11566 and 11567), and M. microtti (ATCC accession no. 11152). Examples of attenuated mycobacterium strains include but are not restricted to M. tuberculosis pantothenate auxotroph strain (i.e. Sambandamurthy J, Nat. Med. 2002; 8(10):1171-1174), M. tuberculosis rpo V mutant strain (i.e. Collins D, et al., Proc. Natl. Acad. Sci. USA 1995; 92(17):8036-8040), M. tuberculosis leucine auxotroph strain (i.e. Hondalus M, et al., Infect. Immun. 2000; 68(5):2888-2898), BCG Danish strain (ATCC accession no. 35733), BCG Japanese strain (ATCC accession no. 35737), BCG, Chicago strain (ATCC accession no. 27289), BCG Copenhagen strain (ATCC accession no. 27290), BCG Pasteur strain (ATCC accession no. 35734), BCG Glaxo strain (ATCC accession no. 35741), BCG Connaught strain (ATCC accession no. 35745), BCG Montreal (ATCC accession no. 35746). In a preferred embodiment, the mycobacterium is a Mycobacterium smegmatis or Mycobacterium bovis. Preferably, the recombinant Mycobacteria spp is Mycobacterium bovis Bacillus Calmette-Guérin (BCG). The recombinant Mycobacterium bovis-BCG has important advantages when used as a vaccine vehicle since (1) it is the only childhood vaccine currently given at birth, (2) in the past 40 years, it has had a very low incidence of adverse effects, when given as a vaccine against tuberculosis; and (3) it can be used repeatedly in an individual (e.g. in multiple forms). More preferably, the recombinant Mycobacteria spp strain is BCG.HIVA^{2auxo} (DSMZ accession no. DSM 26306). See Example 3. The Mycobacteria spp strain BCG.HIVA^{2auxo} is obtained by transforming mycobacterium cells, in particular Mycobacterium bovis BCG cells, with p2auxo.HIVA plasmid of the invention.

The DNA of interest can be integrated or incorporated into the mycobacterial genome and is referred to as integrated DNA or integrated DNA of interest. As a result, the DNA of interest can be introduced stably into and expressed in mycobacteria (i.e. production of foreign proteins is carried out from the DNA of interest present in the mycobacteria). Alternatively, the DNA of interest is integrated into mycobacterial DNA, through the method of the present invention, as a result of homologous recombination. According to the method of the present invention, a recombinant plasmid is used for introduction of the DNA of interest into mycobacterial cells and for stable integration of the DNA into the mycobacterial genome. The recombinant plasmid used includes: 1) mycobacterial sequences (referred to as plasmid-borne mycobacterial sequences) necessary for homologous recombination to occur (between plasmid-borne mycobacterial sequences and sequences in the mycobacterial genome); 2) DNA sequences necessary for replication and selection in E. coli; and 3) the DNA of interest (e.g. DNA encoding a selectable marker and DNA encoding a protein or polypeptide of interest). The recombinant plasmid is introduced, using known techniques, into mycobacterial cells. The mycobacterial sequences in the plasmid can be identical to those present in the mycobacterial genome or sufficiently similar to those present in the mycobacterial genome to make homologous recombination possible. “Recognition” of homology of sequences present in the plasmid-borne mycobacterial DNA and identical of sufficiently similar sequences present in the mycobacterial genome results in crossover between the homologous regions of the incoming (plasmid-borne) mycobacterial DNA and the genomic mycobacterial DNA and integration of the recombinant plasmid into the mycobacterial genome. Integration occurs at a selected site in the mycobacterial genome which is non-essential (i.e. not essential for mycobacterial replication). Integration of the homologous plasmid sequences is accompanied by integration of the DNA of interest into the mycobacterial genome.

The non-virulency of these strains, as well as their ability to colonize the host in the long term, have been studied extensively. Since these mycobacteria are usually used in vivo, it is preferred that the mycobacteria is non-virulent or rendered so by, for example, selecting for non-virulent strains or engineering the mycobacteria to have a mutation or mutations that can fulfil that purpose. Many such mutations are known in the art, such as mutations that render the mycobacterium auxotrophic (e.g. a pan mutation or a lys mutation) or mutations eliminating pathogenicity genes such as an RD1 deletion, as is known in the art. It is also preferred that the mycobacterium utilized for this invention can colonize the host, in order for the mycobacterium to provide a long term antigenic stimulus to the host, thus establishing a strong immune response.

The recombinant cell of the invention typically carries an auxotrophy which can be complemented by the first or second auxotrophic gene. In a particular embodiment, the recombinant cell of the invention has an auxotrophy for glycine. In another embodiment, the recombinant cell of the invention has an auxotrophy for lysine. In a preferred embodiment, the first auxotrophy gene is a gene capable of complementing an E. coli defect in the synthesis of glycine. In a still more preferred embodiment, the auxotrophy gene capable of complementing an E. coli defective in the synthesis of glycine is the GlyA gene. In a preferred embodiment, the second auxotrophy gene is a gene capable of complementing a mycobacterial defect in lysine biosynthesis. In a still more preferred embodiment, the auxotrophy gene capable of complementing a mycobacterial strain defective in the synthesis of lysine is the lysA5 gene.

The recombinant cell of the invention may further comprise a polynucleotide that comprises a sequence encoding a polypeptide of interest. In a preferred embodiment, said polypeptide of interest is an immunogenic polypeptide. In a more preferred embodiment, said polypeptide of interest comprises an immunogenic polypeptide and an endosomalytic polypeptide.

In another embodiment, the polypeptide of interest is a cytokine including, but not limited to, IL-4, IL-5, IL-6, IL-10, IL-12 p40, IL-12 p70, TGFβ and TNFα.

### 5. Pharmaceutical Compositions

The recombinant cells of the invention are particularly useful for the delivery of the polypeptide of interest to a subject in need thereof. Accordingly, when the polypeptide of interest is a polypeptide having an activity of interest, the recombinant cells of the invention are useful as medicaments for the treatment of diseases wherein it is desired to increase in a cell the activity of a polypeptide of interest.

Accordingly, in another aspect, the invention relates to a composition comprising the recombinant cell of the invention. In yet another aspect, the invention relates to a vaccine composition comprising a recombinant cell according to the invention and a pharmaceutically acceptable carrier.

In another aspect, the invention relates to a composition comprising the recombinant cell of the invention for use in medicine. In yet another aspect, the invention relates to a vaccine composition comprising a recombinant cell according to the invention and a pharmaceutically acceptable carrier for its use in medicine. In a particular embodiment, the recombinant cell of the invention lacks a nucleotide sequence, endogenous (as part of the genome of the cell) or exogenous (foreing to the recombinant cell, introduced into the cell by transformation, transduction or conjugation), conferring antibiotic sensitivity or antibiotic resistance to said cell.

The compositions comprising the recombinant bacterium of the present invention may conveniently be presented in unit dosage form. The amount of active ingredient which can be combined with a carrier material to produce a single dosage form will vary depending upon the host being treated, the particular mode of administration. Generally, out of one hundred percent, this dosage will range from about 1 percent to about ninety-nine percent of active ingredient.

Compositions of the invention suitable for oral administration may be in the form of capsules, cachets, pills, tablets, lozenges (using a flavored basis, usually sucrose and acacia or tragacanth), powders, granules, hydrogels, or as a solution or a suspension in an aqueous or non-aqueous liquid, or as an oil-in-water or water-in-oil liquid emulsion, or as an elixir or syrup, or as pastilles (using an inert base, e.g. gelatin and glycerin, or sucrose and acacia), or as mouthwashes, each containing a predetermined amount of the recombinant bacterium as an active ingredient. A compound may also be administered as a bolus, electuary or paste.

In solid dosage forms of the invention for oral administration such as, for example, capsules, tablets, pills, dragees, powders, granules or hydrogels, the active ingredient is mixed with one or more pharmaceutically-acceptable carriers (e.g. sodium citrate or dicalcium phosphate). In the case of capsules, tablets and pills, the pharmaceutical compositions may also comprise buffering agents. Solid compositions of a similar type may also be employed as fillers in soft and hard-filled gelatin capsules using excipients such as, for instance, lactose or milk sugars, or high molecular weight polymers as polyethylene glycol.

The tablets, and other solid dosage forms of the pharmaceutical compositions of the present invention, such as dragees, capsules, pills, granules and hydrogels, may be scored or prepared optionally with coatings and shells, like enteric coatings and other coatings well known in the gallenic art. They may also be formulated so as to provide slow or controlled release of the active ingredient therein using, for example, hydroxypropylmethyl cellulose in varying proportions to provide the desired release profile, other polymer matrices, liposomes, nanoparticles, hydrogels, or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the recombinant bacterium of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers (e.g. ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof). Suspensions, in addition to the active recombinant bacterium may contain suspending agents as, such as, ethoxylated isostearyl alcohols, polyoxyethylene sorbitol and sorbitan esters, microcrystalline cellulose, aluminum metahydroxide, bentonite, agar-agar and tragacanth, and mixtures thereof. Pharmaceutical compositions of the invention for rectal or vaginal administration may be presented as a suppository, which may be prepared by mixing one or more recombinant bacterium with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a recombinant bacteria include powders, sprays, ointments, pastes, creams, lotions, gels, solutions, patches and inhalants. The active recombinant bacteria may be mixed under sterile conditions with a pharmaceutically-acceptable carrier, and with any preservatives, buffers, or propellants which may be required.

The ointments, pastes, creams and gels may contain, in addition to the recombinant bacteria, excipients (e.g. animal and vegetable fats, oils, waxes, paraffins, starch, tragacanth, cellulose derivatives, polyethylene glycols, silicones, bentonites, silicic acid, talc and zinc oxide, or mixtures thereof).

The recombinant bacterium can be alternatively administered by aerosol. This is accomplished by preparing an aqueous aerosol, liposomal preparation or solid particles containing the compound. A non-aqueous (e.g. fluorocarbon propellant) suspension could be used. Sonic nebulizers are preferred because they minimize exposing the agent to shear, which can result in degradation of the compound.

Transdermal patches have the added advantage of providing controlled delivery of a recombinant bacterium to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel. Ophthalmic formulations, eye ointments, powders, solutions and similar eye applications, are also contemplated as being within the scope of this invention.

The pharmaceutical compositions of the present invention suitable for parenteral or intracavity administration comprise one or more recombinant bacteria in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents. In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

### 6. Therapeutic Methods

The recombinant cells of the invention are particularly useful for the generation of an immune response against the immunogenic polypeptide whose expression is under the control of a weak mycobacterium promoter. Thus, the present invention also relates to a method of treating or preventing a disease in a subject in need thereof comprising the administration of the recombinant Mycobacteria spp strain of the invention or the pharmaceutical composition comprising the recombinant Mycobacteria spp strains of the invention. The pharmaceutical compositions according to the present invention comprise vaccines and immunogenic composition containing the recombinant Mycobacteria spp strains of the invention. The invention provides methods for inducing an immune response against an antigenic polypeptide which comprises the administration to a subject in need thereof of a recombinant mycobacterium according to the invention wherein the polypeptide of interest is an antigenic polypeptide.

Accordingly, the invention relates to a recombinant cell according to the invention for its use in the treatment of a disease which requires the expression of a polypeptide of interest. Alternatively, the invention relates to a method for the treatment of a disease that comprises the administration to a subject in need thereof of a recombinant cell according to the invention. In a particular preferred embodiment, the recombinant cell of the invention lacks a nucleotide sequence, endogenous or exogenous, conferring antibiotic sensitivity or antibiotic resistance to said cell.

Similarly, the invention relates to a vaccine composition according to the invention for its use in inducing an immune response against an antigenic polypeptide. Alternatively, the invention relates to a method for the treatment of a disease that comprises the administration to a subject in need thereof of a vaccine according to the invention.

The polypeptide of interest in the composition comprising the recombinant cell of the invention, as well as in the vaccine composition comprising the recombinant cell of the invention, comprises and antigenic polypeptide in such a way that an immune response against the antigenic polypeptide is induced.

The polypeptide of interest comprises an antigenic polypeptide and an endosomalytic polypeptide and wherein the recombinant cell is used in inducing an immune response against the antigenic polypeptide.

Prior to administration to humans as a vaccine, the recombinant mycobacterial strains of the may according to methods well-known in the art. For example, tests for several variables (e.g. toxicity, virulence, safety) are carried out in suitable animal models (e.g. mice, guinea pigs), some of which are usually immunocompromised. The ability of the vaccine preparations to elicit an immune response is tested likewise in suitable animal models (e.g. mice, non-human primates). In addition, protection studies involving vaccination, boosting and subsequent challenges with the live wild type strain are also carried out using suitable animal models (e.g. mice, guinea pigs, non-human primates), so that the contribution of the unspecific protection and immunity conferred by the vector can be depicted. Generally, the dosage employed will be about 10³ to 10¹¹ viable organisms (determined as colony forming units, cfu), preferably about 10⁵ to 10⁹ viable organisms. Alternatively, when infecting individual cells, the dosage of viable organisms to administer will be at a multiplicity of infection ranging from about 0.1 to 10⁶, preferably about 10² to 10⁴.

The amount of the recombinant mycobacteria of the present invention to be administered will vary depending on the species of the subject, as well as the disease or condition that is being treated. Preferably the subject is a mammal. More preferably, the subject is human.

In another embodiment, the subject to which the recombinant mycobacteria and pharmaceutical compositions of the present invention are administered is a non-human mammal. In a preferred embodiment, the subject is a non-human primate, cow, goat, cat, dog, pig, buffalo, badger, possum, deer, or bison. More preferably, the subject is a cow. See O'Reilly L, et al., Tuber. Lung. Dis. 1995; 76(Suppl 1):1-46.

Diseases that may be treated or prevented by using the recombinant mycobacteria and pharmaceutical compositions of the present invention include, but are not limited to, diseases affecting non-human mammals such as bovine tuberculosis, porcine reproductive respiratory syndrome, coccidiosis, leptospirosis, infectious laryngotracheitis, and leishmaniasis. See Wedlock D, et al., Vet. Immunol. Immunopathol. 2011; 144(3-4):220-227, Bastos R, et al., Vaccine 2002; 21:21-29, Wang Q, et al., Vet. Parasitol. 2009; 160(3-4):198-203, K. Seixas F, et al., Biol. Res. 2010; 43:13-18, and Song J, et al., Chin. J. Vet. Sci. 2009; 29(4): 413-417, and Abdelhak S, et al., Microbiology 1995; 141:1585-1592.

In a preferred embodiment, the immunogen is HIV imunogen, in which case the recombinant mycobacteria is used for the treatment of a disease caused by an HIV infection.

In another embodiment, the immunogen is a plasmodium immunogen, in which case the recombinant mycobacteria is used for the treatment of malaria. Suitable plasmodium antigens which may be used in the present invention are sporozoite surface protein 2 (TRAP/SSP2), liver-stage antigen (LSA in particular LSA3), Pf exported protein 1 (Pf Exp1)/Py hepatocyte erythrocyte protein (PyHEP17), and Pf antigen 2 (where Pf represents Plasmodium falciparum and Py represents Plamsodium yoelii), sporozoite and liver stage antigen (SALSA), sporozoite threonine and asparagines-rich (STARP), circumsporozoite protein (CSP), merozoite surface protein 1 (MSP2), in particular merozoite surface protein 1 (MSP-1), merozoite surface protein 2 (MSP-2) merozoite surface protein 3 (MSP-3), merozoite surface protein 4 (MSP-4), merozoite surface protein 6 (MSP-6), Ring-infected erythrocyte surface antigen (RESA), Rhoptry associated protein 1 (RAP-1), Apical membrane antigen 1 (AMA-1), erythrocyte binding antigen (EBA-175), erythrocyte membrane-associated giant protein or antigen 332 (Ag332), dnaK-type molecular chaperone, glutamate-rich protein (GLURP), in particular MSP3-GLURP fusion protein, erythrocyte membrane protein 1 (EMP-1), serine repeat antigen (SERA), clustered-asparagine-rich protein (CARP), cirumsporozoite protein-related antigen precursor (CRA), cytoadherence-linked asexual protein (CLAG), acid basic repeat antigen (ABRA) or 101 kDa malaria antigen, Rhoptry antigen protein (RAP-2), Knob-associated histidine-rich protein (KHRP), Rhoptry antigen protein (RAP), cysteine protease, hypothetical protein PFE1325w, protective antigen (MAg-1), fructose-bisphosphate aldolase, ribosomal phosphoprotein P0, P-type ATPase, glucose-regulated protein (GRP78), asparagine and aspartate-rich protein (AARP1), interspersed repeat antigen or PFE0070w. Antigens of the sexual stage which may be used in the present invention are Sexual stage and sporozoite surface antigen, antigen Pfg27/25, antigen QF122, 11-1 polypeptide, gametocyte-specific surface protein (Pfs230) ookinete surface protein (P25), chitinase, multidrug resistance protein (MRP). The antigens may derive from any of the five species of Plasmodium parasites known to infect human: Plasmodium vivax, Plasmodium ovale, Plasmodium malariae, Plasmodium knowlesi and Plasmodium falciparum.

In another embodiment, the immunogen is a mycobacteria immunogen, in which case the recombinant mycobacteria is used for the treatment of a disease caused by the mycobacteria infection. Suitable mycobacteria immunogen include, without limitation, the 85A, 85B or 85C antigens from Mycobacterium tuberculosis or Mycobacterium bovis or a variant thereof.

Mycobacteria have adjuvant properties and are among the best currently known and stimulate a recipient's immune system to respond to other antigens with great effectiveness. In particular, BCG and other mycobacteria are highly effective adjuvants, and the immune response to mycobacteria has been studied extensively. With nearly 2 billion immunizations, BCG has a well established safety profile in human beings. See Luelmo F, Am. Rev. Respir. Dis. 1982; 125:70-72 and Lotte A, et al., Adv. Tuberc. Res. 1984; 21:107-193. It is one of the few vaccines that can be administered at birth. A valuable aspect of BCG vaccines is that cellular response is elicited. This feature is particularly useful in cases where cell-mediated immunity is considered to be critical for effective treatment, such as in the treatment of neoplastic diseases. Although humoral responses also result, immune protection from mycobacterial infection has been shown to depend on the development of host type-1 T-helper (Th1) cell mediated responses. rBCG can also be effective in stimulating cytotoxic T lymphocytes. Accordingly, in another aspect, the invention relates to the use of recombinant mycobacteria in medicine.

The mycobacteria of the invention are particularly useful for the generation of an immune response against the antigenic polypeptide whose expression is under the control of the weak mycobacterium promoter. Accordingly, the invention provides methods for inducing an immune response against an antigenic polypeptide which comprises the administration to a subject in need thereof of a recombinant mycobacterium according to the invention wherein the polypeptide of interest is an antigenic polypeptide. Alternatively, the invention relates to the use of a recombinant mycobacterium according to the invention wherein the polypeptide of interest is an antigenic polypeptide for inducing an immune response against an antigenic polypeptide. Alternatively, the invention relates to the use of a recombinant mycobacterium according to the invention wherein the polypeptide of interest is an antigenic polypeptide for the manufacture of a medicament for inducing an immune response against an antigenic polypeptide in a subject.

Accordingly, the vaccines are administered in a manner compatible with the dosage formulation, and in such amount as will be therapeutically effective and immunogenic. The quantity to be administered depends on the subject to be treated, capacity of the subject's immune system to generate a cellular immune response, and degree of protection desired. Precise amounts of active ingredient required to be administered depend on the judgment of the practitioner and are peculiar to each individual. However, suitable dosage ranges are of the order of about one microgram to about one milligram, preferably about 1 microgram and more preferably about 5 micrograms, and more preferably 100 micrograms active ingredient per kilogram bodyweight individual. Suitable regimes for initial administration and booster shots are also variable, but are typified by an initial administration followed in one or two week intervals by a subsequent injection or other administration.

The vaccines of the invention may be specially formulated for administration in solid or liquid form, including those adapted for the following: (a) oral administration, such as drenches (aqueous or non-aqueous solutions or suspensions), tablets, boluses, powders, granules, pastes, mouthwash or hydrogels, (b) parenteral administration, for instance, by subcutaneous, intramuscular or intravenous injection of, for example, a sterile solution or suspension, (c) intracavity administration (e.g. intraperitoneal instillation), intravesical (i.e. urinary bladder) instillation, intrathecal administration, (d) intraorgan administration (e.g. intraprostatical administration), (e) topical application (i.e. cream, ointment or spray applied to the skin), (f) intravaginal or intrarectal administration (e.g. as a pessary, cream, foam, enema or suppository) or (g) aerosol (e.g. as an aqueous aerosol, liposomal preparation or solid particles containing the agent(s)).

In vitro assays and animal studies can be used to determine an “effective amount” of the recombinant bacterium, or combinations thereof. A person of ordinary skill in the art would select an appropriate amount of each individual compound in the combination for use in the aforementioned assays or similar assays. Changes in cell activity or cell proliferation can be used to determine whether the selected amounts are “effective amount” for the particular combination of compounds. The regimen of administration also can affect what constitutes an effective amount. Further, several divided dosages, as well as staggered dosages, can be administered daily or sequentially, or the dose can be proportionally increased or decreased as indicated by the exigencies of the therapeutic situation.

The use of a heterologous booster vaccine to bolster immunity elicited by BCG has gained attention recently. Thus, BCG-primed laboratory animals and humans develop impressive cellular immune responses following a heterologous boost comprised of modified vaccinia Ankara (MVA) encoding Mtb antigen 85A, hereinafter “Ag85A”, also known as Rv3804c. See Vordemeier G H, et al., Immunol. 2004; 112(3):461-470 and McShane H, et al., Nature Med. 2004; 10(11):1240-1244. In contrast, naive individuals develop relatively unimpressive responses to the MVA-Ag85A vector. See McShane, 2004, supra. Accordingly, the immunity generated by the vaccine compositions of the invention can be improved if said vaccine is administered together with a DNA-based vaccine encoding the antigenic polypeptide or an immunologically active epitope thereof which forms part of the recombinant mycobacteria of the invention. Therefore, the present invention, in a preferred embodiment, further relates to composition comprising the vaccine according to the present invention and a DNA-based vaccine which includes a sequence encoding the same peptide which is expressed by the recombinant mycobacterium or an immunologically active epitope thereof.

In a preferred embodiment, the DNA based vaccine may be in the form of “naked” DNA vaccines. In case of “naked” DNA vaccines, the construction of DNA based vaccines comprises the cloning of the polynucleotide encoding at least one antigenic polypeptide or an immunologically active epitope thereof into an expression cassette under the control of an adequate promoter, including a viral promoter such as the cytomegalovirus immediate early promoter. An advantage of DNA immunization is that both cellular (including CD4⁺ and CD8⁺ T cells) and humoral immune responses can be induced. Peptide epitopes encoded by the naked DNA vaccine are presentable by major histocompatibility complexes (MHC) class I as well as class II complexes after processing into peptides by the proteasome. The MHC class I-peptide of the invention complex can be recognized by CD8⁺ T cells and can induce CD8⁺ T cells to acquire antigen-specific cytotoxic functions. These CD8⁺ cytotoxic T lymphocytes (CTL) should then be able to kill cells with a peptide of the invention presented on their surface, such as a melanoma cell with HERV-K-MEL on its surface. Naked DNA vaccines may be administered directly by intramuscular or intradermal injections of the expression vectors, by using a needle-free injection device or a with gold particles using a gene gun coated with DNA into the target tissue, by in vivo electropermeabilization, such as by intramuscular administration as polymer-based formulations of DNA followed by electroporation of the injected muscle and the use of aerosols.

In an embodiment of the invention, the vaccine composition can further comprise a vector encoding the antigenic polypeptide or an immunologically active epitope thereof. In particular, the vector comprising the antigen of interest or an immunogenic epitope thereof is selected from the group consisting of modified vaccinia virus Ankara vector, an adenoviral vector and a measles virus vector.

The DNA based vaccine could be in the form of a recombinant viral vaccine. In this case the DNA with the expression cassette for the recombinant protein is integrated into a viral genome, the recombinant viral vector. A single round of a self-limited infection with such a recombinant virus can be sufficient to elicit a broad immunity. For the generation of recombinant viral vaccines, several viruses are particularly useful including, without limitation, modified vaccinia virus Ankara (MVA), canary-pox virus, adenovirus, Sindbis virus, dengue virus, yellow fever virus, measles virus, sendai virus, polio virus, papilloma virus, rota virus, Venezuelan encephalitis virus, Semliki forest virus and hepatitis A virus. Preferred are vectors of the pox group, papilloma, Venezuelan encephalitis virus, Semliki forest virus, adeno virus and hepatitis A virus. In all embodiments relating to a DNA based vaccine of the invention, it is preferred that in the DNA-based vaccine the polynucleotide encodes 5 to 30 copies, preferably 8 to 25, more preferably 10 to 20 copies of the peptide of the invention, such as defined in I) or II above, or 2 to 10 copies, more preferably 4-8 copies, of the polypeptide of the invention as defined above, for example a polypeptide comprising a peptide of the invention as defined above, such as defined in I) or II above. Preferably the multiple copies of said peptide or said polypeptide are encoded in the context of a single fusion polypeptide. In a preferred embodiment, the DNA-based vaccine is selected from a modified vaccinia virus Ankara (MVA), an adenoviral vector or a measles virus vector.

Compositions comprising the vaccine of the present invention and one or more DNA-based vaccines encoding the same antigen as the vaccine of the invention can be used in medicine or for inducing an immune response against said antigen in a patient in need thereof. The different components of the compositions of the present invention may be administered together. However, the most effective immune responses with BCG-based vaccines combined with DNA-based vaccines can be obtained at present by a combination of two different applications. Accordingly, in one embodiment, the vaccine of the invention and the vector comprising the polynucleotide encoding the antigen of interest or an immunogenic epitope thereof are administered in a separate or sequential manner using a prime-boost strategy.

In a still more preferred embodiment, the administration involves a priming step with the host cell of the invention followed by one or more boosting step(s) with the DNA-based vaccine encoding the antigen of interest or an immunogenic epitope thereof.

The methods of the present invention can comprise administering one or more (a plurality) doses of the priming composition, followed by one or more doses of the first boosting composition to induce an immune response. In a particular embodiment, both the priming composition and boosting composition are administered in multiple doses.

The methods of the present invention also can comprise administering one or more (a plurality) doses of the priming composition to induce an immune response. In one embodiment, the priming composition is administered in multiple doses. In a particular embodiment, the priming composition is administered twice.

The timing of the individual doses will depend on the individual. For example, the timing of the priming and boosting doses can be in the region of from about one week to three weeks, about 6 weeks to 9 weeks, about 9 weeks to 12 weeks, about 12 weeks to 15 weeks, about 15 to about 18 weeks and about 18 weeks to about 21 weeks apart. In particular embodiments, the timing of the priming and boosting doses can be about 2 weeks, 3 weeks, 4 weeks, 5 weeks, 6 weeks, 7 weeks, 8 weeks, 9 weeks, 10 week, 11 weeks, 12, weeks, 13 weeks, 14, weeks, 15 weeks, 16 weeks, 17 weeks, 18 weeks, 19 weeks, 20 weeks, 21 weeks, 22 weeks, 23 weeks, 24 weeks or 25 weeks apart.

### 7. In Vitro Expression Methods of the Invention

The present invention reveals also that the concomitant use of a weak mycobacterium promoter, an auxotrophy/complementing gene and an auxotrophic mycobacterial host strain favors the stabilization of a genetic construct containing an heterologous gene in the mycobacterial strain, thus resulting in long-term expression in the cells of the protein encoded by said heterologous gene.

Thus, in another aspect, the invention relates to a method for the expression of a polypeptide of interest in a mycobacterium host cell which comprises:
(i) growing a mycobacterium host cell comprising a polynucleotide according to the invention and, optionally
(ii) recovering the polypeptide of interest from the culture,
   wherein the host cell carries an auxotrophy which can be complemented by said auxotrophic gene.

In a first step, the method for the expression of a polypeptide of interest in a mycobacterium host cell comprises growing a mycobacterium host cell comprising a sequence encoding the polypeptide of interest under the operative control of a weak mycobacterium promoter under conditions adequate for expression of the polypeptide of interest.

In a preferred embodiment, the weak mycobacterium promoter is the Mycobacteria spp α-antigen promoter or a functionally equivalent variant thereof.

In another embodiment, the sequence encoding the polypeptide of interest is fused in frame to a sequence encoding a signal sequence active in mycobacteria. In a stimm lore preferred embodiment, the signal sequence corresponds to the signal sequence of Mycobacterium tuberculosis 19 KDa lipoprotein or a functionally equivalent variant thereof.

In another embodiment, the polypeptide of interest comprises an endosomalytic polypeptide. In a still more preferred embodiment, the endosomalytic polypeptide comprises Listeria monocitogenes listeriolysin, Clostridium perfringens perfingolysin, Mycobacterium tuberculosis phospholipase C or a variant thereof.

In another embodiment, the polynucleotide of interest is an immunogenic polypeptide. In a more preferred embodiment, the immunogenic polypeptide comprises a HIV polypeptide, a Mycobacteria spp polypeptide, a Plasmodium falciparum epitope, or an immunologically active epitope thereof. In a still more preferred embodiment, the HIV polypeptide is gp120 or HIVA.

Typically, the strains are obtained by transforming the mycobacterial host strain of choice with the E. coli/mycobacterial expression vector. Such transformation is carried out by methods known in the art including electroporation, facilitated delivery, transformation using cationic lipid complexes or transformation using particle-mediated or pressure-mediated delivery.

Typically, the vector contains a selectable marker, such as an auxotrophy marker, an antibiotic resistance gene (e.g. tetracycline, rifampicin, ampicillin, kanamycin) or a mercury resistance, which allows the selection of those recombinant cells which have incorporated the vector.

Alternatively, the vector comprising the sequence encoding the polypeptide of interest under the operative control of a weak mycobacterium promoter further comprises
i) a first auxotrophy complementing gene which confers an auxotrophic host strain carrying said gene the capability of growing in a first medium that lacks a first auxotrophic factor or
ii) a second auxotrophy complementing gene which confers an auxotrophic host strain carrying said gene the capability of growing in a second medium that lacks a second auxotrophic factor.

The skilled person will appreciate that, if the strain has been obtained by transformation with an episomal plasmid containing the gene encoding the polypeptide of interest and the first and second auxotrophic genes, then the first and second auxotrophy genes will form part of the same vector which contains the polynucleotide encoding the gene of interest. However, when the strains have been obtained by transformation with an integrative plasmid containing the gene encoding the polypeptide of interest and the first and second auxotrophic genes, the auxotrophic genes will be typically also integrated in the genome of the host cell.

The strains carrying said first and second auxotrophic complementing genes, either as extrachromosomal genetic elements or integrated in the genome can be selected by growing the cells in a medium lacking said first or said second auxotrophic factor. In a preferred embodiment, the vector comprises said first and second auxotrophic genes in which case the cells can be selected from the presence of the vector encoding the gene of interest by growing them in the absence of said first or second auxotrophic factors or in the absence of both auxotrophic factors.

In a preferred embodiment, the first auxotrophy gene is an E. coli gene capable of complementing an E. coli glycine auxotrophy. In a still more preferred embodiment, the gene capable of complementing an E. coli glycine auxotrophy is the glyA gene. In another preferred embodiment, the second auxotrophy gene is a gene capable of complementing a lysine auxotrophy. In a still more preferred embodiment, the gene capable of complementing a mycobacterium lysine auxotrophy is the mycobacterium lysA gene. Preferably, the mycobacterium lysA gene is the mycobacterium lysA5 gene.

The terms “adequate conditions” refer to those conditions of time, temperature and culture media which allows continued growth of the mycobacterial strains. Typically, expression of the protein may be achieved by incubating the mycobacterial cells, preferably, BCG or Mycobacterium smegmatis, in Middlebrook 7H9 medium (Difco™, BD Biosciences, Inc., Franklin Lakes, N.J., US) or in Sauton medium at 37° C. See Yamada T, et al., J. Bacteriol. 1987; 169:839-843. In addition, the growth rate of BCG can be enhanced by the addition of oleic acid (0.06 percent v/v), or the addition of 10% of Middlebrook ADC supplement (5 g/L albumin, 2 g/L dextrose, 0.003 g/L catalase final concentration, BD Biosciences, Inc., Franklin Lakes, N.J., US), and detergents (e.g. Tyloxapol (0.05 percent v/v)) or 0.05% Tween 80 which are used to prevent cell clumping. The purity of BCG cultures can be evaluated by evenly spreading 100 μL of the BCG culture serially diluted (e.g. 10-fold steps from Neat to 10⁻⁸) in phosphate buffered saline (herein referred to PBS) onto 10 cm plates containing 25-30 mL of solid media (e.g. supplemented Middlebrook 7H10). In addition, the purity of the culture can be further assessed using commercially available kits such as thioglycolate medium and soybean-casein medium (catalog no. 211768, BD Biosciences, Inc., Franklin Lakes, N.J., US).

Wherein the strains comprises one or more auxotrophic genes, the culture media is preferably minimal medium lacking the auxotrophic factor which deficiency can be complemented with the expression of the auxotrophic complementing gene. Suitable minimal medium for growing mycobacteria include, without limitation, the minimal media described by Youmans and Karlson comprising L-asparagine, potassium dihydrogen phosphate, potassium sulphate, citric acid, magnesium carbonate, glycerol, at pH of 7.2; the minimal medium described by Ratledge and Hall; Middlebrook 7H10 medium for solid medium, 7H9 for liquid broth, optionally supplemented with 10% ADC or 10% OADC (ADC plus oleic acid), or the Mahenthiralingam minimal medium. ee Youmans G, et al., Am. Rev. Tuberc. Pulm. Dis. 1947; 55:529-535, Ratledge C, et al., FEBS Letters 1970; 10:309-312, and Mahenthiralingam E, et al., J. Gen. Microbiol. 1993; 139:575-583. These media, used without lysine complementation, could be considered as lys defective, and are suitable to be used as selective media. In a preferred embodiment, the method for the expression of a polypeptide of interest according to the invention involves the use of a mycobacteria strain auxotrophic for lysine, in particular a Mycobacterium bovis BCG strain and, even more preferably, a mycobacterium strain deficient in the lysA gene and carrying the lysA5 gene which are then cultured in minimal media lacking lysine.

If secretion of the expressed polypeptide from the mycobacteria strain is desired, the sequence encoding the antigenic polypeptide is fused in frame to a sequence coding a signal sequence active in mycobacteria. In a preferred embodiment, the signal sequence corresponds to the signal sequence of Mycobacterium tuberculosis 19 KDa lipoprotein or a functionally equivalent variant thereof, wherein said term has been described in detail herein above in the context of the polynucleotide of the invention.

In order to detect whether the strains are producing the polypeptide, it is possible to analyze the reactivity between the protein collected from the supernatant of the culture and the antibody recognizing the intended foreign antigen by western blotting method. See Borremans M, et al., J. Bacteriol. 1988; 170:3847-3854. As the antibody, usable is a rabbit serum (polyclonal antibody) to be obtained by immunizing a rabbit with the intended foreign antigen peptide or an antigen protein containing said peptide along with an adjuvant. Alternatively, also usable is a monoclonal antibody to be obtained by selecting a cell line that produces an antibody reactive with the intended foreign antigen peptide from the hybridoma obtained by fusing the spleen cells of the immunized mouse and myeloma cells by an ordinary method, followed by incubating the thus-selected cell line. See Koehler G, et al., Eur. J. Immunol. 1976; 6:511-519. In the antigen-antibody reaction, if the reactivity between the fusion protein and the antibody is higher than that between the original carrier protein not fused with the intended foreign antigen peptide and the antibody, it is judged that said fusion protein has the antigenicity derived from the intended foreign antigen peptide. The protein content of the culture can be analyzed by polyacrylamide gel electrophoresis (PAGE), where the proteins present in the culture are separated according to its molecular weight. The peptide of interest can be detected by means of a western blot, where the proteins from the electrophoresis gel are transferred to a more stable membrane, and submitted to an antigen-antibody staining procedure. It is also possible to confirm the antigen-producing capacity of BCG of the present invention using an animal model, the intended foreign antigen peptide or an antigen protein containing said peptide is supported on a suitable solid phase, and the reaction between this and the anti-serum of an animal immunized with BCG could be assessed by various methods known in the art (e.g. enzyme-linked immunosorbent assays (ELISA), western blot, dot-blot or indirect immunofluorescence).

Once sufficient expression of the polypeptide of interest has been achieved, step (ii) of the method for the expression of a polypeptide of interest in a mycobacterium host cell comprises the recovery of the polypeptide of interest from the culture. The skilled person will appreciate that the recovery step will vary depending on whether the polypeptide is produced intracellularly or whether the polypeptide is secreted by means of a signal sequence.

In the case that the polypeptide of interest is expressed intracellularly, step (ii) of the method of the invention requires separating the cells from the culture supernatant, usually by centrifugation or filtration, followed by lysis of the cells. Host cells can be lysed using any standard method including chemical/enzymatic cell lysis, mechanical lysis, thermal cycling lysis, boiling lysis, electrochemical lysis, electroporation lysis, and ultrasonic lysis. Once the cells are lysed, the sample is treated in order to remove cell debris and unbroken cells, typically by centrifugation until a cell-free lysate containing the polypeptide of interest is obtained.

The cell-free lysate obtained as defined in the previous paragraph (if the polypeptide of interest is expressed intracellularly) or the cell culture supernatant (if the polypeptide of interest is secreted) is then subjected to one or more protein purification steps in order to isolate the polypeptide of interest. Suitable protein purification methods include, without limitation, size fractionation using molecular sieve chromatography; ion-exchange chromatography; affinity chromatography using, for instance, monoclonal antibodies directed to the polypeptide of interest, adsorption chromatography using nonspecific supports, such as hydroxyapatite, silica, alumina, selective precipitation, and the like. The fractions obtained during the above purification procedures are then assayed for the presence of the polypeptide of interest. The identification of the polypeptide of interest after the fractionation can be established using a number of methods known in the art, including but not limited to SDS-PAGE, western blotting, and mass spectrometry assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, fluorescence polarization immunoassay (FPIA), nephelometric inhibition immunoassay (NIA), agglutination assays, complement fixation assays, immunofluorescence assays, protein A assays, and immunoelectrophoresis assays. See Stites D, et al., “Medical Immunology”, 9^{th} Ed. (Appleton & Lange, Norwalk, Conn., US, 1997), Asai D, Ed., “Methods in Cell Biology: Antibodies in Cell Biology”, Vol. 37 (Academic Press, Waltham, Mass., US 1993), and Harlow E, et al., Eds. “Antibodies-A Laboratory Manual” (Cold Spring Harbor Laboratory, Cold Spring Harbor, N.Y., US, 1988).

This invention is further illustrated by the following examples which should not be construed as limiting. The contents of all references, patents and published patent applications cited throughout this application are incorporated herein in their entirety by reference.

### Example 1

### Construction of Double Auxotrophy Complementing Polynucleotides

1. Bacterial Strains and Culture Methods

The Mycobacterium bovis BCG Pasteur lys auxotrophic strain was developed according protocols previously described. See Pavelka, 1999, supra. The E. coli M15Δgly A strain was constructed according to Vidal, 2008, supra. The deletion of chromosomal glyA locus was performed by using the one-step chromosomal gene inactivation technique. See Datsenko K, et al., Proc. Natl. Acad. Sci. USA 2000; 97:6640-6645. This method is based on a λ red recombinase that facilitates the recombination of linear PCR products into the chromosome of E. coli.

Escherichia coli DH5α cultures were grown in Luria-Bertani (LB) broth or on LB agar plates at 37° C., using ampicillin resistance as a selectable marker. E. coli M15 Δgly cultures were grown in M9 minimal medium supplemented with glucose 0.4%, MgSO₄ 2 mM, CaCl₂ 0.1 mM (broth or agar) at 37° C. Mycobacterial cultures were grown in Middlebrook 7H9 broth (Difco™, BD Biosciences, Inc., Franklin Lakes, N.J., US) or on Middlebrook agar 7H10 medium (Difco™, BD Biosciences, Inc., Franklin Lakes, N.J., US) supplemented with glycerol, albumin-dextrose-catalase (ADC, Difco™, BD Biosciences, Inc., Franklin Lakes, N.J., US) and containing 0.05% Tween 80. The L-lysine monohydrochloride (Sigma-Aldrich Co., Saint Louis, Mo., US) was dissolved in distilled water and used at a concentration of 40 μg/mL. The L-glycine (Sigma-Aldrich Co., Saint Louis, Mo., US) was dissolved in distilled water and used at a concentration of 40 μg/mL. See Table 2.

TABLE 2
Bacterial strains

| | Relevant | |
|---|---|---|
| Bacterial strains | characteristics | Reference |
| Mycobacterium bovis | Pasteur | Pavelka, 1999, supra |
| BCG mc² 1604 | ΔlysA5::res | |
| Escherichia coli DH5α | | Catalogue number 11319-019 |
| | | Invitrogen ®, Life |
| | | Technologies Corp., |
| | | Carslbad, CA, US |
| E.coli M15Δgly | Glycine | Vidal, 2008, supra |
| | auxotrophy | |

2. E. coli-Mycobacterial Shuttle Plasmid pJH222.HIVA Kanamycin Resistance+(pHIVACAT1)

The parental plasmid pJH222 has been described previously. See Cayabyab C, et al., J. Virol. 2006; 2:1645-1652. The plasmid DNA pJH222.HIVA expressing the HIVA immunogen was constructed according to protocols known in the art. See Joseph J, et al., J. Biomed. Biotechnol. 2010; 2010:357370 and Im, 2007, and Saubi, 2011, supra. The E. coli/mycobacterial shuttle vector pJH222.HIVA (8060 bp) was used as parental plasmid for plasmid DNA constructs. The pJH222.HIVA shuttle is a replicative vector (multicopy extrachromosomal) containing a DNA cassette encoding kanamycin resistance, an E. coli origin of replication (oriE), and an expression cassette containing a mycobacterial promoter, a multiple cloning site and a transcriptional terminator. In addition, the pJH222 vector includes a mycobacterial plasmid DNA origin of replication (oriM) and the lysA complementing gene under the regulatory control of BCG hsp60 promoter. The entire DNA coding sequence of HIVA was synthesized by PCR, using oligonucleotide primers specific for HIVA gene and cloned into pJH222 Escherichia coli/mycobacterial shuttle vector under the regulatory control of Mycobacteria spp α-antigen promoter. The HIVA gene is fused to the 5′ region encoding the 19 KDa lipoprotein signal sequence from Mycobacterium tuberculosis. The HIVA immunogen is a derived from consensus HIV-1 clade A Gag protein, derived from an HIV-1 strain prevalent in central and eastern Africa, and a string of CD8+-T cell epitopes. See Hanke, 2000, supra. The cloning of the HIVA gene into the plasmid DNA is confirmed by DNA sequencing, PCR and enzyme restriction analysis performed following standardized protocols known in the art. See Brown T, “Gene Cloning” (Chapman & Hall, London, GB, 1995), Watson R, et al., “Recombinant DNA”, 2nd Ed. (Scientific American Books, New York, N.Y., US, 1992), Alberts B, et al., “Molecular Biology of the Cell” (Garland Publishing Inc., New York, N.Y., US, 2008), Innis M, et al., Eds., “PCR Protocols. A Guide to Methods and Applications” (Academic Press Inc., San Diego, Calif., US, 1990), Erlich H, Ed., “PCR Technology. Principles and Applications for DNA Amplification” (Stockton Press, New York, N.Y., US, 1989), Sambrook J, et al., “Molecular Cloning. A Laboratory Manual” (Cold Spring Harbor Laboratory Press, Cold Spring Harbor, N.Y., US, 1989), Bishop T, et al., “Nucleic Acid and Protein Sequence. A Practical Approach” (IRL Press, Oxford, GB, 1987), Reznikoff W, Ed., “Maximizing Gene Expression” (Butterworths Publishers, Stoneham, Mass., US, 1987), Davis L, et al., “Basic Methods in Molecular Biology” (Elsevier Science Publishing Co., New York, N.Y., US, 1986), and Schleef M, Ed., “Plasmid for Therapy and Vaccination” (Wiley-VCH Verlag GmbH, Weinheim, D E, 2001).

3. pQEαβFucA Plasmid

pQEαβFucA is a derivative of pQEαβ-rham plasmid DNA. The pQEαβ-rham plasmid was developed as previously described. See Vidal L, et al., Microbial Cell Factories 2006; 5(Suppl 1):P85. This plasmid derived from the pQE40 commercial vector (Qiagen NV, Venlo, NL). The glyA gene (hereinafter referred to as “β fragment”) from E. coli K-12 was fused to the weak constitutive promoter P3 (hereinafter referred to as “α fragment”) in this vector. The fusion αβ product was cloned into pQErham, a pQE40-derived vector for ramnulose 1-phosphate aldolase (RhuA) overexpression. The resulting complementation vector, pQEαβ-rham, was used to transform the E. coli M15ΔglyA host strain. The glyA gene encodes for serine hydroxymethyl transferase (SHMT), an enzyme involved in the main glycine biosynthesis pathway. This gene was deleted from the E. coli M15 strain to obtain the auxotrophic strain.

### Example 2

### Construction of E. coli-Mycobacterial Expression Vector (p2auxo.HIVA) Containing the E. coli Glycine Complementing Gene and Mycobacterial Lysine Complementing Gene

1. Release of the Kanamycin Resistance Gene from pJH222HIVA Vector (pHIVACAT1)

The plasmid pJH222.HIVA was digested with SpeI restriction enzyme. The SpeI targets flank the kanamycin resistance gene. The resulting product after SpeI digestion was treated with calf intestinal alkaline phosphatase (CIAP), to prevent auto-annealing of the larger fragment. Then, the digestion+CIAP treated product was subjected to an agarose gel electrophoresis. The large band obtained, of approximately 6,431 bp, was cut and extracted from the agarose gel, using standard procedures. A linearized plasmid with sticky ends containing all plasmid components but the kanamycin resistance gene was thus obtained.

2. Amplification of the E. coli glyA Gene by PCR

The pQEαβFucA contains the glyA gene and its promoter sequence, also known as αβ (MroI-XbaI/1662 bp DNA fragment). See Vidal, 2008, supra. PCR primers were designed to amplify the αβ fragment incorporating additional transcriptional terminator sequences (αβT1). Primers were designed to include SpeI and SmaI restriction enzyme targets at both ends of the αβT1 PCR fragment with the following structure:
SmaISpeIαβT1SpeISmaI

The primers utilized for incorporation of the SmaI and SpeI enzyme sites were:

Forward: SpeISmaIFwdglyA:
SEQ ID NO: 029
5′-TCACCCGGGACTAGTTGCTCATCCGGAGTGAAGAC-3′
Reverse: SpeISmaIRevglyA:
SEQ ID NO: 030
5′-ACGCCCGGGACTAGTTCTAGAGGGCGGATTTGTCCTAC-3′

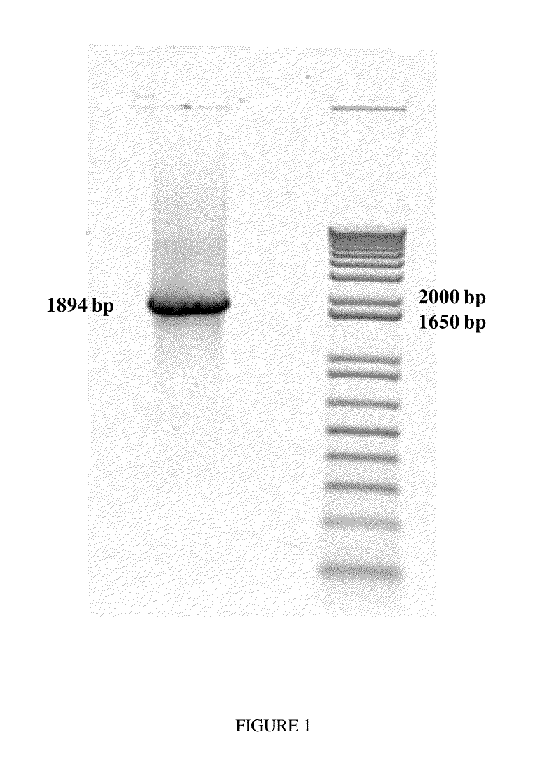

The PCR performed using the aforementioned primers rendered the product SmaISpeIαβT1SpeISmaI. See FIG. 1.

3. Ligation of SmaISpeIαβT1SpeISmaI PCR Product into pNEB193 Vector

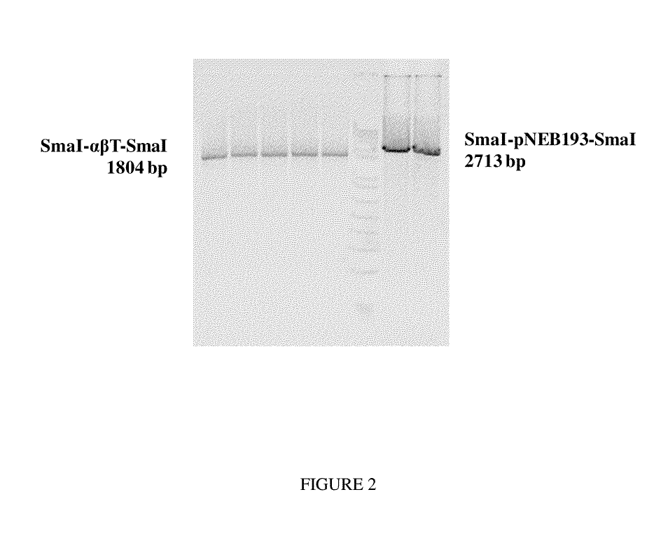

The SmaISpeIαβT1SpeISmaI PCR product was digested with SmaI restriction enzyme. The pNEB193 plasmid was also treated with the same SmaI restriction enzyme. The resulting digestion products were gel purified and ligated ON at 16° C. See FIG. 2. The ligations were transformed in E. coli DH5α cells and plated out on LB agar plates, containing ampicillin 50 μg/mL. 20 colonies were screened and grown in LB broth. Plasmid DNAs were purified and analyzed in an electrophoresis gel. Only one E. coli colony (L2-4) showed the presence of the plasmid DNA.

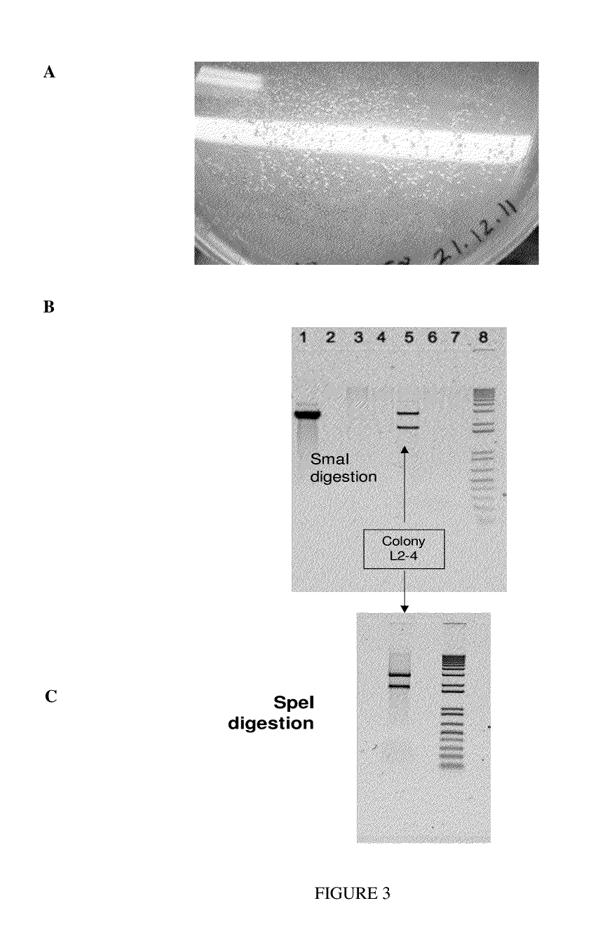

The plasmid DNA (pNEB193-αβT1) from E. coli (colony L2-4) was analyzed by SpeI and SmaI digestion and the expected bands and digestion profile were observed. See FIG. 3.

4. Ligation of the SpeIαβT1SpeI Insert into pHIVACAT1

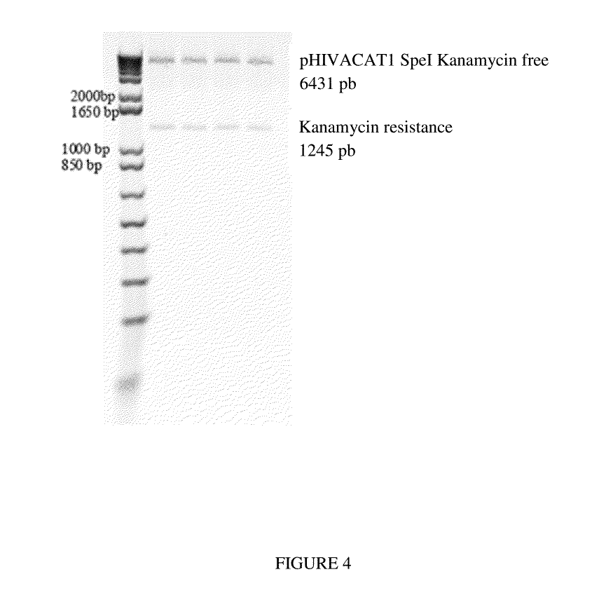

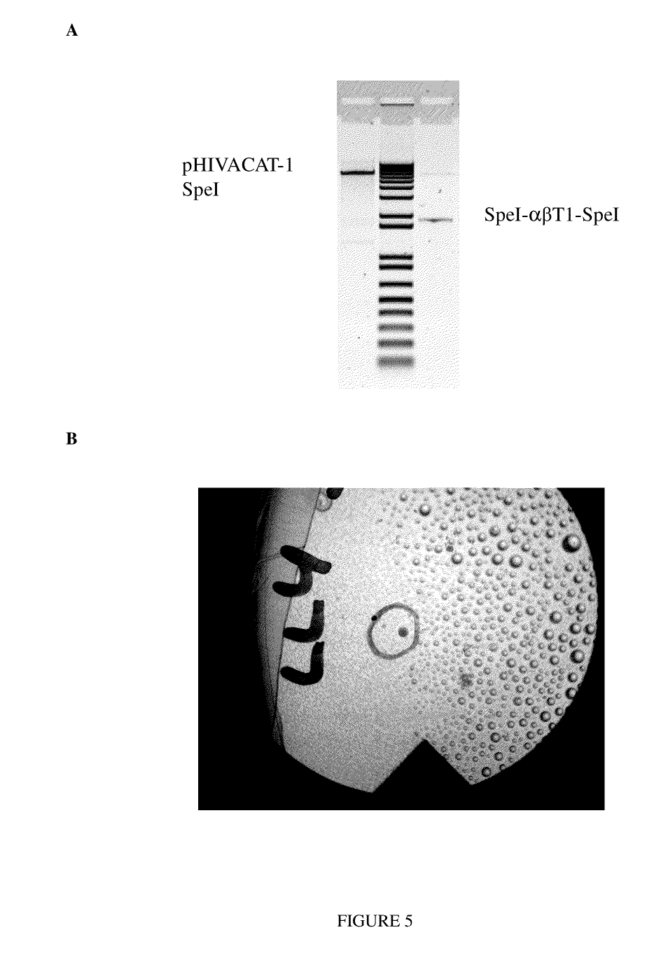

The SpeI-αβT1-SpeI fragment obtained from the pNEB193-αβT1 plasmid DNA was used for cloning into the pHIVACAT1 plasmid DNA. The band corresponding to the insert (which had SpeI flanking sites) was purified and ligated to the SpeI digested large fragment of pHIVACAT1 vector. See FIGS. 3C and 4. The SpeI-pJH222HIVA-SpeI band and the SpeI-αβT1-SpeI were ligated using the T4 DNA ligase, at 4° C. O/N. E. coli M15 Δgly cells were transformed by electroporation, and plated out onto M9 agar plates (glycine defective) at 37° C. for 48 h. See FIGS. 5A and 5B.

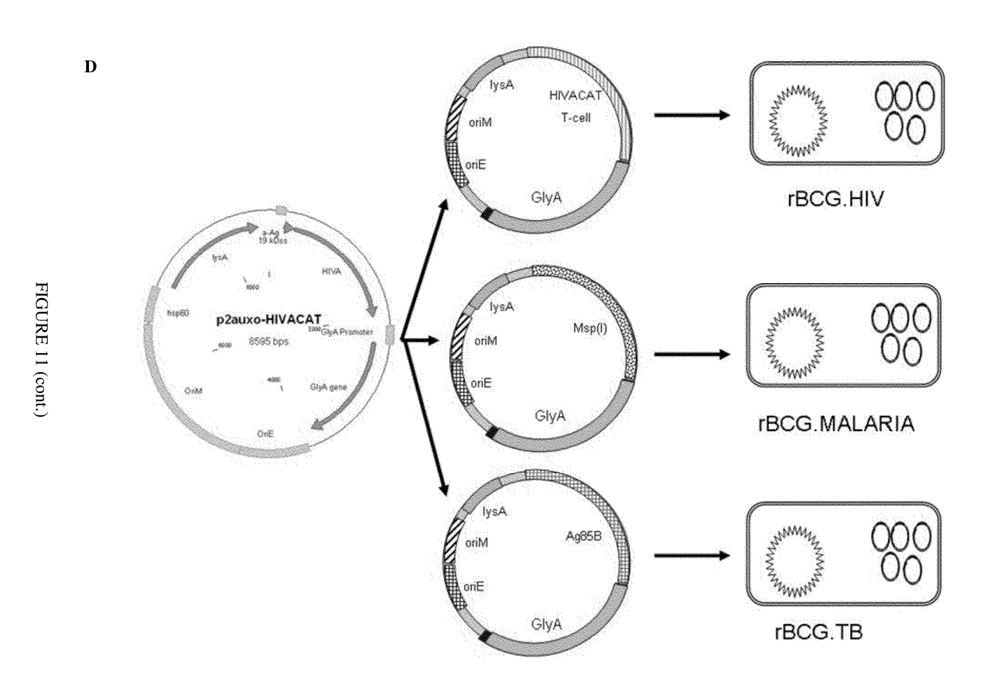

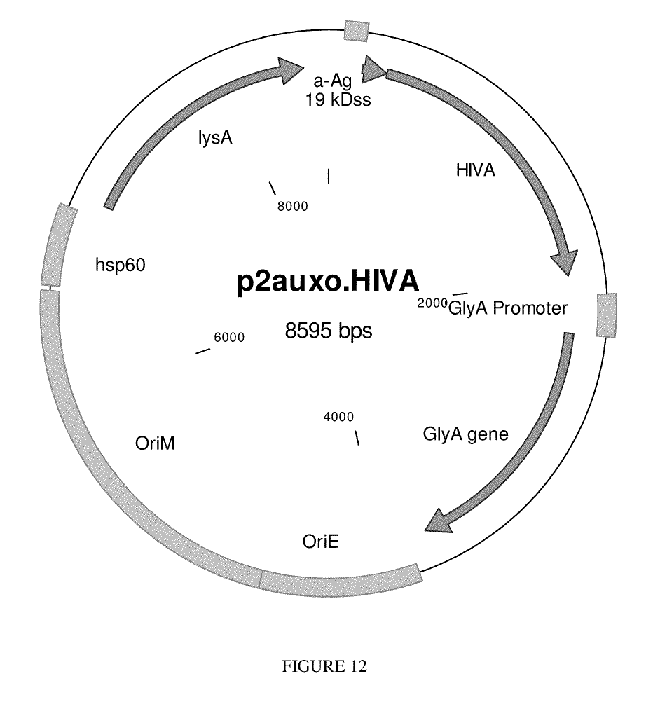

One E. coli colony was observed. The confirmation of positive recombinant E. coli M15Δgly colony harboring the ligation product, from now on p2auxo.HIVA plasmid, was performed by SpeI digestion, restriction enzyme profile and PCR of the HIVA DNA fragment. See FIGS. 8 and 12.

### Example 3

### Construction of Recombinant BCG Harbouring the p2auxo.HIVA Plasmid DNA: BCG.HIVA^{2auxo}

1. Mycobacteria transformation with p2auxo.HIVA plasmid DNA by electroporation

The BCG lysine auxotroph was transformed by electroporation. See Pavelka, 1999, supra. The BCG lysine auxotroph was transformed with p2auxo.HIVA (kanamycin minus, glycine plus, lysine plus). BCG lys-cultures were grown (in 7H9 medium supplemented with glycerol. ADC, Tween 80 and lysine 40μ/mL) to an OD of 0.9 (600 nm) and pelleted at 3,000 rpm. The pellets were washed twice by resuspension and centrifugation (3,000 rpm) in 10% glycerol at 4° C. and finally resuspended to 1/20 of the original culture volume in cold 10% glycerol. Then, 100 μL of the cold BCG suspension were mixed with plasmid DNA (50-500 ng) in a pre-chilled 0.2 cm electroporation cuvette and transformed using the Biorad Gene Pulser electroporator at 2.5 kV, 25 mF, and 1,000Ω. After electroporation, 1 mL of 7H9 medium (Difco™, BD Biosciences, Inc., Franklin Lakes, N.J., US), supplemented with glycerol and albumin-dextrose-catalase (ADC, Difco™, BD Biosciences, Inc., Franklin Lakes, N.J., US) and containing 0.05% Tween 80 (Sigma-Aldrich Co., Saint Louis, Mo., US), was added and incubated at 37° C. for 12 hours before plating on Middlebrook agar 7H10 medium (Difco™, BD Biosciences, Inc., Franklin Lakes, N.J., US) supplemented with glycerol, ADC (Difco™, BD Biosciences, Inc., Franklin Lakes, N.J., US) and containing 0.05% Tween 80 (Sigma-Aldrich Co., Saint Louis, Mo., US). The plates were incubated at 37° C. for three weeks.

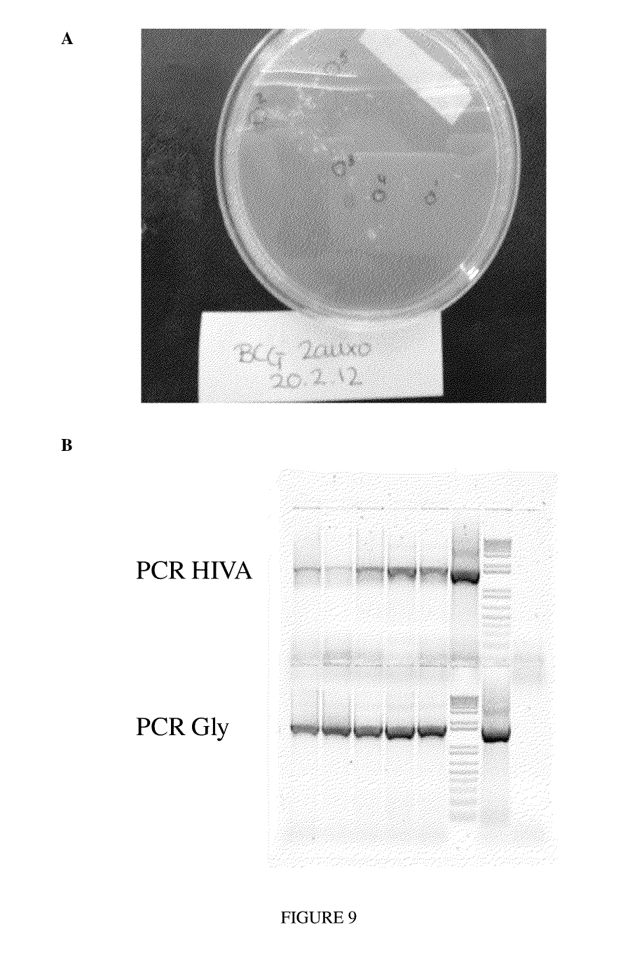

The BCG.HIVA² colonies obtained were grown and analyzed for plasmid DNA integrity, by detecting the presence of the HIVA and glyA DNA coding sequence by BCG colonies PCR. See FIG. 9.

Plasmid DNA p2auxo.HIVA is a replicative (multicopy, extrachromosomal) vector that contains a DNA cassette encoding, an E. coli origin of replication (oriE) and a mycobacterial plasmid DNA origin of replication (oriM). It also contains the wild-type glycine A-complementing gene (glyA) and lysine A-complementing gene (lysA5) for the vector selection and maintenance in the E. coli and BCG auxotroph strain respectively. p2auxo.HIVA is an antibiotic resistance-free plasmid.

2. Immunodot Analysis

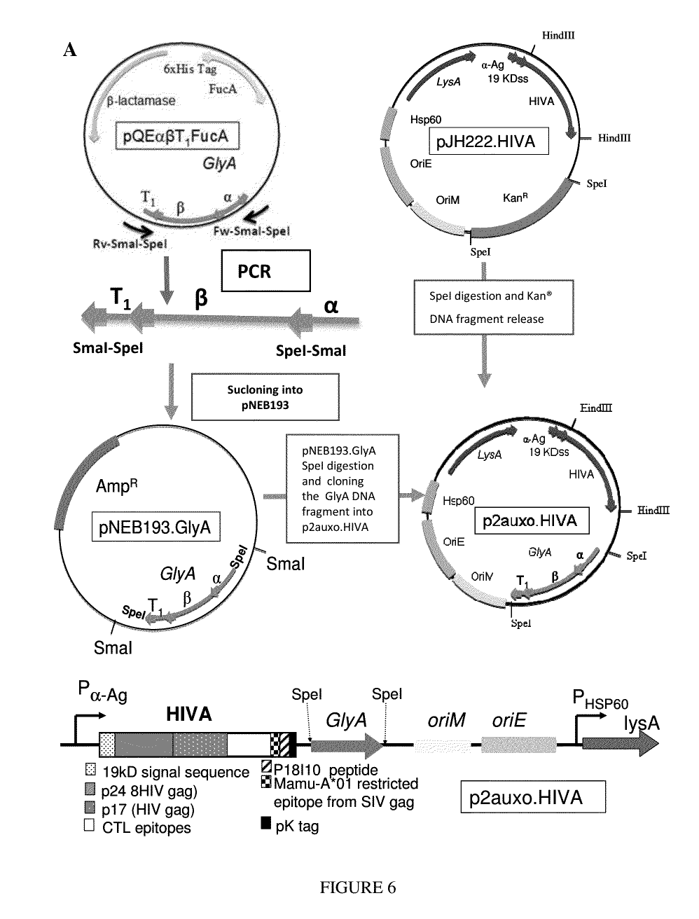

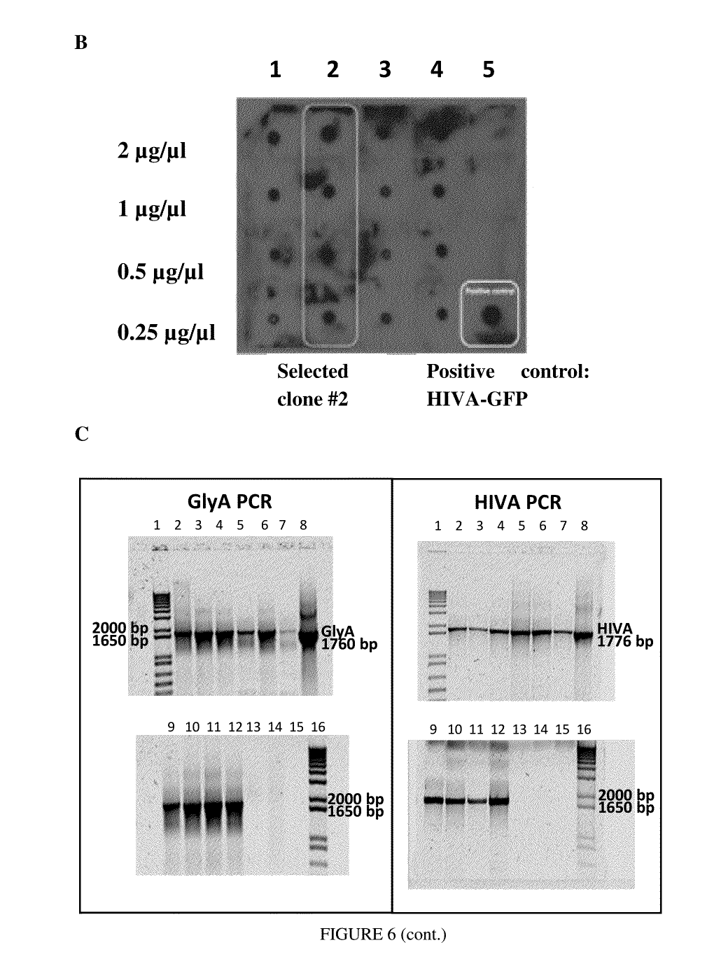

BCG transformants were grown to mid-logarithmic phase in liquid 7H9 (Difco™, BD Biosciences, Inc., Franklin Lakes, N.J., US) medium supplemented with glycerol and albumin-dextrose-catalase (ADC, Difco™, BD Biosciences, Inc., Franklin Lakes, N.J., US) and containing 0.05% Tween 80. Alternatively, an OADC supplement could be used. rBCG cultures were centrifuged at 3,000 rpm for 10 minutes at 4° C. Pellets were washed twice in PBS plus 0.02% Tween-80 and resuspended in 1 mL of extraction buffer (50 mM Tris-HCl pH 7.5, 5 mM EDTA, 0.6% sodium dodecyl sulfate). 5 μL of 100× protease inhibitor cocktail (1 mg/mL aprotinin, 1 mg/mL E-64, 1 mg/mL leupeptin, 1 mg/mL pepstatin A, 50 mg/mL pefabloc SC, and 10 mL DMSO) was then added. Cells were sonicated for 4 cycles of 1 minute on ice with a Branson sonicator at output control four (max output for the micro tip used), duty cycle 50%. Extracts were centrifuged at 13,000 rpm for 10 minutes at 4° C. and supernatants were collected. The cell lysates supernatants were loaded to a suitable membrane (i.e. nitrocellulose, PVDF). The membranes were probed using an anti-Pk antibody, a murine monoclonal antibody, MCA 1360, directed against a paramyxovirus derived sequence present in the HIVA immunogen that was utilized as an in vitro marker. See Hanke, 1992, supra. The MCA 1360 antibody was detected using a suitable secondary antibody (i.e. goat anti mouse, ProteinG) labeled with, horse raddish peroxidase (HRP). The bound HRP was visualized with an ECL kit a luminol based chemoluminiscence detection method (Pierce, USA). To visualize the dots, the Typhoon 8600 gel imaging system was used (GE Healthcare, General Electric Co., USA). See FIG. 6b.

The BCG.HIVA^{2auxo} clone that showed higher HIVA protein expression by immunodot analysis (clone #2) was selected for seed lots preparation. The resulting mycobacteria stock, with an antibiotic-free plasmid selection system, was prepared as “Master Seed” and “Working Vaccine Stock” for conducting preclinical assays. The final product was labeled as BCG.HIVA^{2auxo}.

3. Genetic Characterization of the BCG.HIVA^{2auxo}

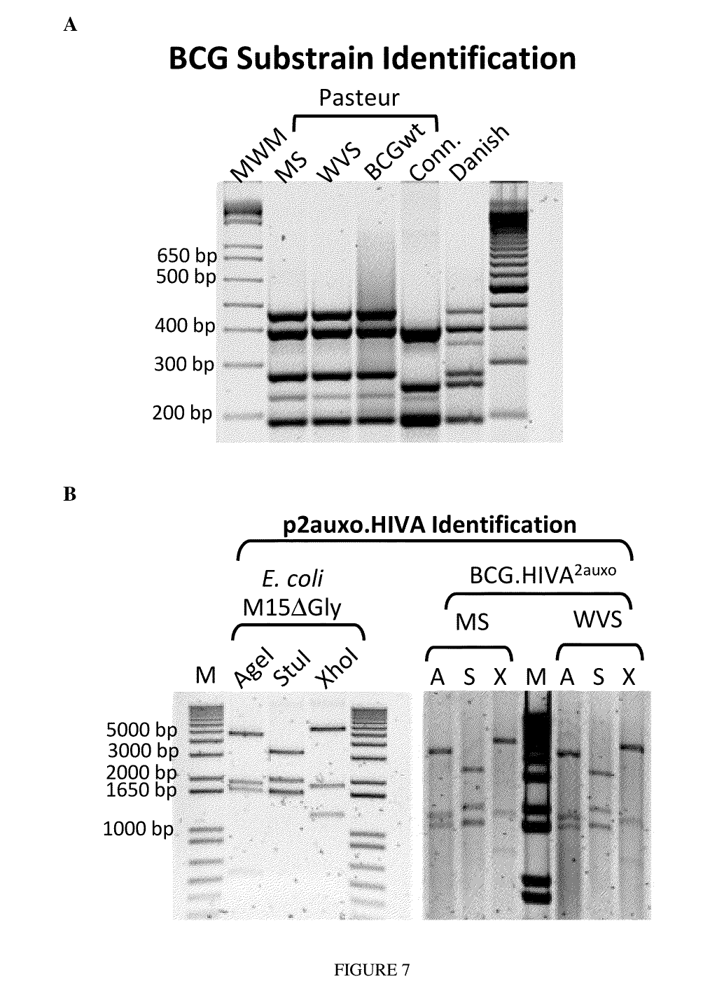

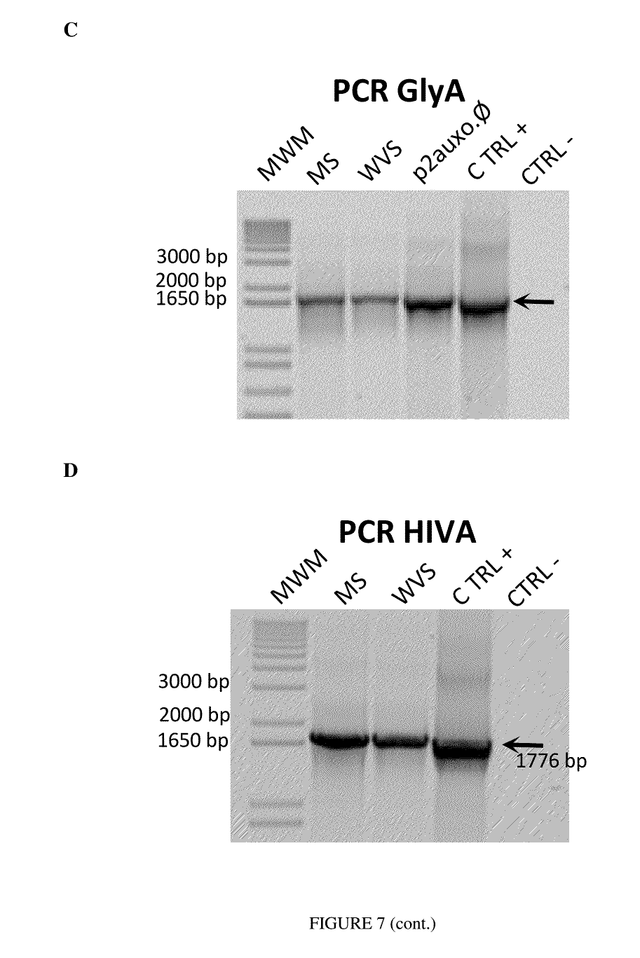

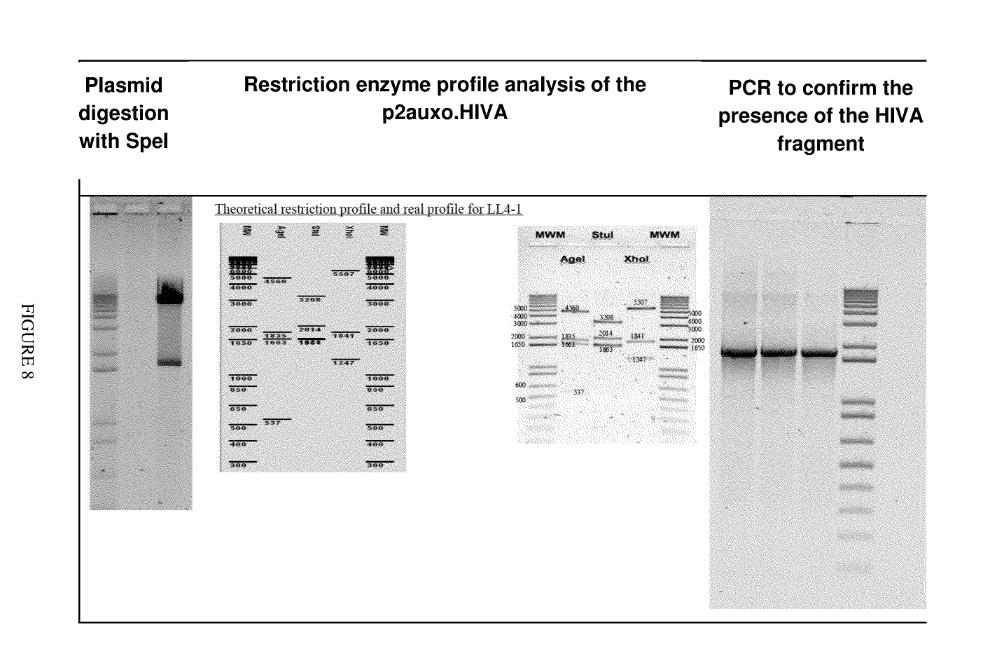

In order to confirm that the BCG.HIVA^{2auxo} vaccine strain correspond to BCG Pasteur substrain, the method described by Bedwell et al. (Bedwell J et al. 20010 Vaccine 19(15-26): 2146-2151). based on multiplex PCR system targeting SenX3-RegX3 system and the BCG deletion regions including RD1, 2, 8, 14 and 16. The following samples were tested: BCG.HIVA^{2auxo} strain (clone 2) Pasteur substrain, BCG wild type Pasteur, commercial BCG Connaught and BCG Danish 1331 strain. For the PCR analysis, 5 μl of the mycobacterial DNA isolated from BCG.HIVA^{2auxo} Pasteur, BCG Danish 1331 and BCG Connaught strains was as used in a final volume of 50 μl. The PCR fingerprints of BCG Pasteur, BCG Danish and BCG Connaught substrains were consistent with previously published results on genetic information of BCG substrains. All BCG substrains evaluated gave a 196 bp product with primers ET1-3, indicating deletion of the RD1 region. In addition, the RD8 and RD16 regions were present in BCG Pasteur (BCG.HIVA^{2auxo}) and gave a product of 472 and 401 bp, respectively. The primers for the SenX3-RegX3 region gave a product of 276 bp in BCG Pasteur. The PCR fingerprints of BCG Pasteur, BCG Connaught and BCG Danish substrains (FIG. 7A) were consistent with previously published results on genetic information of BCG substrains.

For the molecular characterization of p2auxo.HIVA plasmid DNA, enzymatic restriction and PCR analysis were performed. The plasmid DNA was isolated from the Master seed and Working stock of BCG.HIVA^{2auxo} strain and was characterized. The enzymatic restriction pattern obtained did not show any difference with the predicted enzymatic pattern of the plasmid DNA sequence isolated from E. coli (pre-BCG transformation) (FIG. 7B). On the other hand, the PCR analysis using specific primers for the HIVA and E. coli glyA DNA coding sequences was performed using the BCG liquid culture from BCG.HIVA^{2auxo} Master seed and Working stock as template. A band of 1760 bp and 1776 bp corresponding to E. coli glyA and HIVA DNA fragment, respectively, were detected (FIGS. 7C and 7D).

4. Phenotypic Characterization of the BCG.HIVA^{2auxo}

In this study, a glycine auxotroph of the E. coli M15 strain and a lysine auxotroph of BCG strain complemented with a glycine and lysine gene were used, as well as an antibiotic-free plasmid selection system. The phenotype stability of glycine and lysine auxotrophy, glycine and lysine complementation and kanamycin sensitivity of E. coli and BCG.HIVA^{2auxo} strains were assessed. On the one hand, the E. coli glycine auxotrophic strain failed to grow on non glycine supplemented agar plates (FIG. 10A), while growing on agar plates supplemented with glycine (FIG. 10B). As expected, complementation of E. coli M15ΔglyA strain with glyA gene provided on the multicopy plasmid p2auxo.HIVA abolished the requirement for exogenous glycine (FIG. 10C). Also, when E. coli M15ΔglyA strain was plated out on agar plates containing kanamycin, no colonies were observed (FIG. 10D), confirming the lack of kanamycin resistance in the construct of the invention. On the other hand, BCG lysine-auxotrophic strain failed to grow on non lysine supplemented agar plates (FIG. 10E), while growing on agar plates supplemented with lysine (FIG. 10F). As expected, complementation of BCG.HIVA^{2auxo} strain with lysine gene provided on the multicopy plasmid p2auxo.HIVA abolished the requirement for exogenous lysine (FIG. 10G). In addition, when BCG.HIVA^{2auxo} strain was plated out on agar plates containing kanamycin, no colonies were observed (FIG. 10H), confirming the lack of kanamycin resistance in the construct according to the invention.

### Example 4

### BCG.HIVA^{2auxo} Activity

The recombinant Mycobacterium bovis BCG expressing the HIVA immunogen by means of the p2auxo.HIVA DNA shuttle vector was inoculated to BALB/c mice. BALB/c is a suitable model for immunogenicity testings of HIVA related vaccine models. As part of the HIVA DNA coding sequence, there is an epitope, P18I10 which is immunodominant for H2^{d} murine HLA (BALB/c).

1. BCG.HIVA^{2auxo} Prime and MVA.HIVA Boost Regimen Elicited HIV-1-Specific CD8+ and PPD-Specific T-Cell Responses in Mice

Adult (7-weeks-old) female BALB/c mice were either left unimmunized or immunized with BCG.HIVA^{2auxo} or BCG wild type and were boosted with MVA.HIVA at doses, routes and schedules outlined in the corresponding figure legends. On the day of sacrifice, individual spleens were collected and splenocytes were isolated by pressing spleens through a cell strainer (Falcon) using a 5-ml syringe rubber plunger. Following the removal of red blood cells with ACK lysing buffer (Lonza), the splenocytes were washed and resuspended in complete medium (R10 [RPMI 1640 supplemented with 10% fetal calf serum and penicillin-streptomycin], 20 mM HEPES, and 15 mM 2-mercaptoethanol).

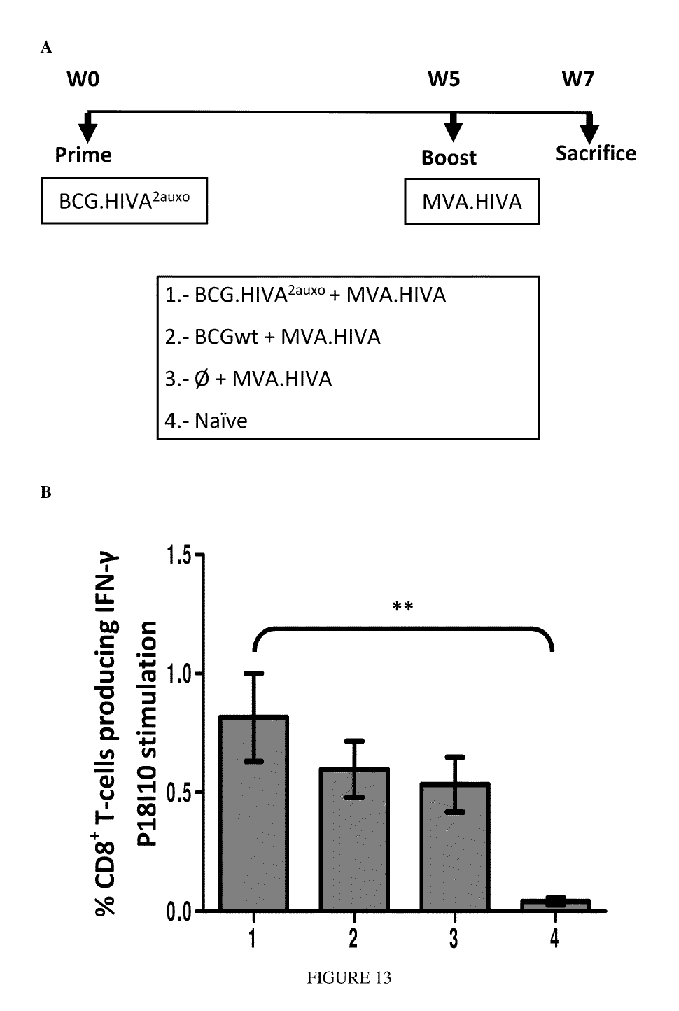

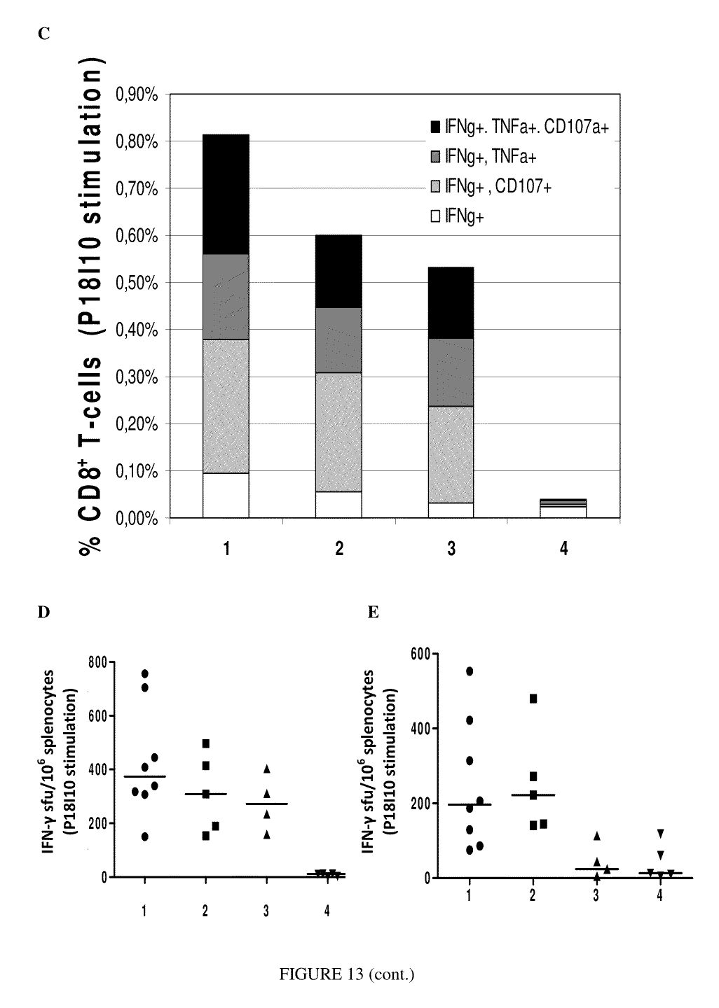

The specific HIV-1 T-cell immune responses in BALB/c mice after immunization with BCG.HIVA^{2auxo} prime and MVA.HIVA boost were evaluated. The immunogenicity readout was focused on the P18-I10 epitope, an immunodominant CTL epitope derived from HIV-1 Env and H-2Dd murine restricted, which was fused to HIVA immunogen to evaluate the immunogenity in mice (FIG. 13A). The functional specific T cells in response of peptide stimulation were measured by intracellular cytokine staining (ICS) and ELISPOT assays. It was observed that BCG.HIVA^{2auxo} prime and MVA.HIVA boost elicited the highest proportion of P18-I10 epitope specific CD8⁺ T-cells producing IFN-γ, compared with the BCG wild type priming and MVA.HIVA boost and with MVA.HIVA alone (FIG. 13B). On the other hand, the quality of the elicited CD8+ T-cells in terms of their ability to produce IFN-γ, TNF-α, and to degranulate (surface expression of CD107a) in response to P18-I10 peptide stimulation was also investigated. It was found that BCG.HIVA^{2auxo} prime and MVA.HIVA boost induced higher frequencies of trifunctional specific CD8+ T-cells compared with the BCG wild type priming and MVA.HIVA boost and with MVA.HIVA alone (FIG. 13C). The capacity of splenocytes from vaccinated mice to secrete IFN-γ was tested also by ELISPOT assays. The highest frequency of specific cells secreting IFN-γ was observed in mice primed with BCG.HIVA^{2auxo} and boosted with MVA.HIVA (FIG. 13D).

BCG.HIVA^{2auxo} elicited PPD-specific responses in mice. The BCG-specific immune responses were assessed following the vaccine regimen consisting of BCG.HIVA^{2auxo} prime and MVA.HIVA boost as described in FIG. 13A. The capacity of splenocytes from vaccinated mice to secrete IFN-γ was tested by ELISPOT assays. The splenocytes secreted IFN-γ after overnight stimulation with the PPD antigen. The median spot-forming units (SFU) per 10⁶ spenocytes were similar in mice primed with BCG.HIVA^{2auxo} or BCG wild type (196 and 222 sfu/million splenocytes respectively) (FIG. 13E).

2. BCG.HIVA^{2auxo} Prime and MVA.HIVA Boost was Well Tolerated in Mice

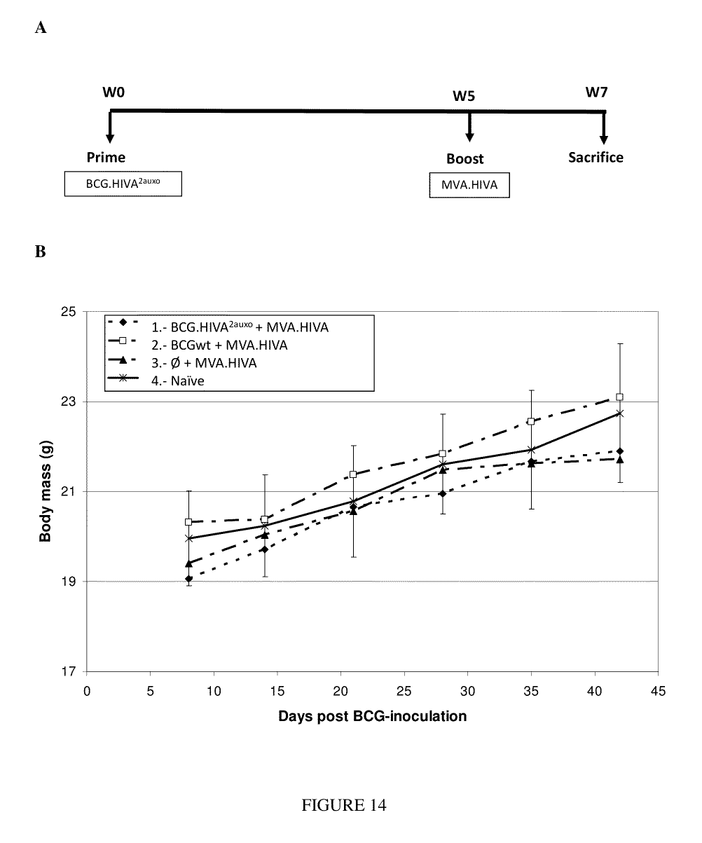

As shown in FIG. 14B, the body mass was weekly monitored and recorded. All vaccine combinations were analyzed, to depict any possible adverse events due to vaccination and monitored by body mass loss. Importantly, no statistically significant difference was observed between the vaccinated mice groups and the naïve mice group. Furthermore, between week 0 and week 7, the body mass monitored in all vaccinated mice groups was found between the mean±1 standard deviation (SD) body mass curve in naïve mice (FIG. 14B). It is also important to mention that no mice died during the trial, no local adverse events, and no associated systemic reactions were observed.

### Example 5

### Construction of Additional Double Auxotrophy Complementing Polynucleotides: p2auxo. CSP, p2auxo.Ag85B and p2auxo.HIVc(G+C)

The CSP immunogen DNA coding sequence was provided by Professor Adrian Hill from Jenner Institute. First, the plasmid DNA p2auxo.HIVA was digested by HindIII restriction enzyme, and the HIVA DNA coding sequence was released. Second, the CSP immunogen DNA coding sequence, was amplified by PCR using specific primers and HindIII extension sites and was inserted into p2auxo vector.

The Ag85 DNA coding sequence was provided by Professor Kazuhiro Matsuo from Japan BCG laboratory. First, the plasmid DNA p2auxo.HIVA was digested by HindIII restriction enzyme, and the HIVA DNA coding sequence was released. Second, the Ag85B immunogen DNA coding sequence, was amplified by PCR using specific primers and HindIII extension sites and was inserted into p2auxo vector.

The HIVc DNA sequence was provided by Professor Tomas Hanke from Jenner Institute and was BCG codon optimized, synthesized in vitro and cloned into pGH plasmid DNA (Biomatik, USA). First, the plasmid DNA p2auxo.HIVA was digested by HindIII restriction enzyme, and the HIVA DNA coding sequence was released. Second, the HIVc immunogen DNA coding sequence was released by HindIII digestion and inserted into p2auxo vector.

HIVc DNA sequence corresponds to SEQ ID NO:031.

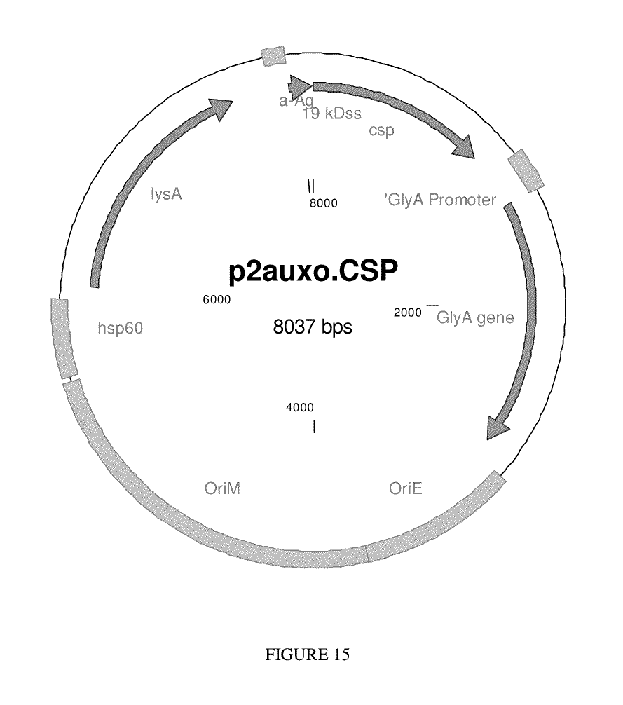

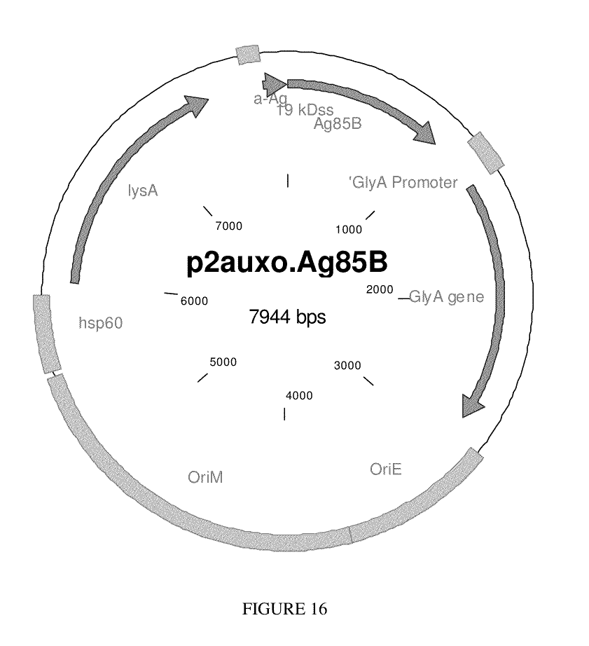

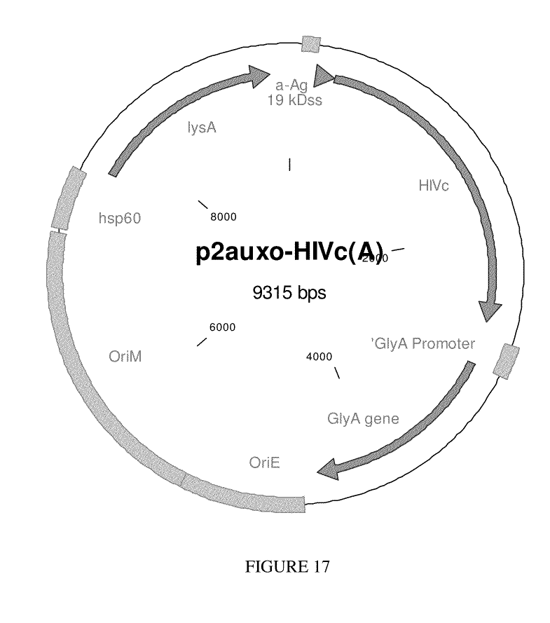

p2auxo.CSP, p2auxo.Ag85B and p2auxo.HIVc(G+C) are shown, respectively, in FIGS. 15, 16 and 17.

1-47. (canceled)

48. A polynucleotide comprising:
(i) a sequence encoding a polypeptide of interest,
(ii) a first auxotrophy complementing gene which confers an auxotrophic host strain carrying the gene the capability of growing in a medium that lacks a first auxotrophic factor, and
(iii) a second auxotrophy complementing gene which confers an auxotrophic host strain carrying the gene the capability of growing in a medium that lacks a second auxotrophic factor,
wherein the polynucleotide does not comprise any nucleotide sequence conferring antibiotic sensitivity or antibiotic resistance.

49. The polynucleotide of claim 48 further comprising a mycobacterial origin of replication.

50. The polynucleotide of claim 49, wherein the mycobacterial origin of replication is oriM.

51. The polynucleotide of claim 49 further comprising a second origin of replication.

52. The polynucleotide of claim 51, wherein the second origin of replication is oriE.

53. The polynucleotide of claim 48, wherein the sequence encoding the polypeptide of interest is under the operative control of a heterologous weak mycobacterium promoter.

54. The polynucleotide of claim 52, wherein the sequence encoding the polypeptide of interest is under the operative control of a heterologous weak mycobacterium promoter.

55. The polynucleotide of claim 53, wherein the weak mycobacterium promoter is the Mycobacteria spp α-antigen promoter or a functionally equivalent variant thereof.

56. The polynucleotide claim 48, wherein the sequence encoding the polypeptide of interest is fused in frame to a sequence encoding a signal sequence active in mycobacteria and/or wherein the polypeptide of interest comprises an immunogenic polypeptide and an endosomalytic polypeptide.

57. The polynucleotide of claim 56, wherein the signal sequence corresponds to the signal sequence of Mycobacterium tuberculosis 19 kDa lipoprotein or a functionally equivalent variant thereof, wherein the immunogenic polypeptide comprises a HIV polypeptide, a Mycobacteria spp polypeptide, a Plasmodium falciparum epitope, a Plasmodium berghei epitope or an immunologically active epitope thereof or wherein the endosomalytic polypeptide comprises Listeria monocitogenes listeriolysin, Clostridium perfringens perfingolysin, Mycobacterium tuberculosis phospholipase C or a variant thereof.

58. The polynucleotide of claim 57, wherein the HIV polypeptide is gp120 or HIVA or HIV-c, wherein the Mycobacteria spp polypeptide is Ag85B from Mycobacterium bovis BCG or wherein the Plasmodium berghei epitope is circumsporozoite protein (CSP) from Plasmodium berghei.

59. The polynucleotide of claim 48, wherein the first auxotrophy gene is a gene capable of complementing a mycobacterium lysine auxotrophy or the second auxotrophy gene is an E. coli gene capable of complementing E. coli glycine auxotrophy.

60. The polynucleotide of claim 59, wherein the gene capable of complementing a mycobacterium lysine auxotrophy is the lysA gene or wherein the gene capable of complementing an E. coli glycine auxotrophy is the glyA gene.

61. A vector comprising the polynucleotide of claim 48.

62. A bacterium comprising the polynucleotide of claim 48.

63. The bacterium of claim 62, wherein the bacterium does not comprise any endogenous or exogenous nucleotide sequence conferring antibiotic sensitivity or antibiotic resistance to the bacterium.

64. The bacterium of claim 62, wherein the polynucleotide encodes a polypeptide of interest, wherein the polypeptide comprises an antigenic polypeptide.

65. A method for the treatment of disease selected from the group consisting of a disease caused by a HIV infection, malaria, and a disease caused by a mycobacteria infection, comprising administering to a subject in need thereof, a bacterium comprising a polynucleotide, the polynucleotide comprising:
(i) a sequence encoding a polypeptide of interest, wherein the polypeptide of interest comprises an antigenic polypeptide,
(ii) a first auxotrophy complementing gene which confers an auxotrophic host strain carrying the gene the capability of growing in a medium that lacks a first auxotrophic factor, and
(iii) a second auxotrophy complementing gene which confers an auxotrophic host strain carrying the gene the capability of growing in a medium that lacks a second auxotrophic factor,
wherein the polynucleotide does not comprise any nucleotide sequence conferring antibiotic sensitivity or antibiotic resistance, wherein the bacterium does not comprise any endogenous or exogenous nucleotide sequence conferring antibiotic sensitivity or antibiotic resistance to the bacterium, and wherein if the method is for the treatment of a disease caused by a HIV infection, the polypeptide of interest comprises an HIV immunogen, if the method is for the treatment of malaria, the polypeptide of interest comprises a plasmodium immunogen or if the method is for the treatment of disease caused by a mycobacteria infection, the polypeptide of interest comprises an immunogen from a mycobacteria.

66. A vaccine composition comprising
(i) a bacterium comprising a polynucleotide, the polynucleotide comprising:
(a) a sequence encoding a polypeptide of interest, wherein the polypeptide of interest comprises an antigenic polypeptide,
(b) a first auxotrophy complementing gene which confers an auxotrophic host strain carrying the gene the capability of growing in a medium that lacks a first auxotrophic factor, and
(c) a second auxotrophy complementing gene which confers an auxotrophic host strain carrying the gene the capability of growing in a medium that lacks a second auxotrophic factor,
wherein the polynucleotide does not comprise any nucleotide sequence conferring antibiotic sensitivity or antibiotic resistance and
wherein the bacterium does not comprise any endogenous or exogenous nucleotide sequence conferring antibiotic sensitivity or antibiotic resistance to the bacterium,
and
(ii) a pharmaceutically acceptable carrier.

67. A method for the expression of a polypeptide of interest in a mycobacterium host cell which comprises:
(i) growing a mycobacterium host cell comprising a polynucleotide according to claim 48 and, optionally,
(ii) recovering the polypeptide of interest from the culture,
wherein the host cell carries an auxotrophy which can be complemented by at least one of the auxotrophic genes forming part of the polynucleotide.


SEQUENCE LISTING (33 sequences)

SEQ ID NO: 1 - protein, 9 aa, HIV-1
ALKHRAYEL

SEQ ID NO: 2 - protein, 9 aa, HIV-1
PPIPVGEIY

SEQ ID NO: 3 - protein, 9 aa, HIV-1
GEIYKRWII

SEQ ID NO: 4 - protein, 10 aa, HIV-1
KRWIILGLNK

SEQ ID NO: 5 - protein, 10 aa, HIV-1
FRDYVDRFYK

SEQ ID NO: 6 - protein, 11 aa, HIV-1
RDYVDRFYKTL

SEQ ID NO: 7 - protein, 9 aa, HIV-1
DRFYKTLRA

SEQ ID NO: 8 - protein, 9 aa, HIV-1
ALFQSSMTK

SEQ ID NO: 9 - protein, 9 aa, HIV-1
ITLWQRPLV

SEQ ID NO: 10 - protein, 9 aa, HIV-1
ERYLKDQQL

SEQ ID NO: 11 - protein, 8 aa, HIV-1
YLKDQQLL

SEQ ID NO: 12 - protein, 10 aa, HIV-1
TVYYGVPVWK

SEQ ID NO: 13 - protein, 11 aa, HIV-1
RPQVPLRPMTY

SEQ ID NO: 14 - protein, 10 aa, HIV-1
QVPLRPMTYK

SEQ ID NO: 15 - protein, 8 aa, HIV-1
VPLRPMTY

SEQ ID NO: 16 - protein, 9 aa, HIV-1
AVDLSHFLK

SEQ ID NO: 17 - protein, 9 aa, HIV-1
DLSHFLKEK

SEQ ID NO: 18 - protein, 8 aa, HIV-1
FLKEKGGL

SEQ ID NO: 19 - protein, 9 aa, HIV-1
ILKEPVHGV

SEQ ID NO: 20 - protein, 10 aa, HIV-1
ILKEPVHGVY

SEQ ID NO: 21 - protein, 9 aa, HIV-1
HPDIVIYQY

SEQ ID NO: 22 - protein, 9 aa, HIV-1
VIYQYMDDL

SEQ ID NO: 23 - protein, 10 aa, HIV-1
TPGPGVRYPL

SEQ ID NO: 24 - protein, 11 aa, HIV-1
ACTPYDINQML

SEQ ID NO: 25 - protein, 10 aa, HIV-1
RGPGRAFVTI

SEQ ID NO: 26 - protein, 21 aa, Mycobacterium tuberculosis
MKRGLTVAVAGAAILVAGLSG

SEQ ID NO: 27 - DNA, 50 bp, Mycobacterium tuberculosis
ccgaggtaaggactgagcatgggcccgataaagtgactattatggatttc

SEQ ID NO: 28 - DNA, 119 bp, Mycobacterium tuberculosis
tctagaatacggaaatgagacgactttgcgcccgaatcgacatttggcctccacacacggtatgttctggcccgagcacacgacgacatacaggacaaaggggcacaggtatgcatatg

SEQ ID NO: 29 - DNA, 35 bp, Artificial
  SpeISmaIFwdGlyA primer
tcacccgggactagttgctcatccggagtgaagac

SEQ ID NO: 30 - DNA, 38 bp, Artificial
  SpeISmaIRevGlyA primer
acgcccgggactagttctagagggcggatttgtcctac

SEQ ID NO: 31 - DNA, 2373 bp, HIV
ggaggcaagaagcttgaggagaaggccttcagccctgaggtgatccccatgttcaccgccctgtccgagggcgccaccccccaggacctgaacaccatgctgaacaccgtgggcggccaccaggccgccatgcagatgctgaaggacaccatcaacgaggaggccgccgagtgggaccgcatctacaagcgctggatcatcctgggcctgaacaagatcgtgcgcatgtactcccccgtgtccatcctggacatccgccagggccccaaggagcccttccgcgactacgtggaccgcttcgcccgcaactgccgcgcccctcgcaagaagggctgctggaagtgcggcaaggagggccaccagatgaaggactgcaccgagcgccaggccaacttcctgggcaagatctggccctcccgctggaagcccaagatgattggcgggatcggcggcttcatcaaggtgcgccagtacgaccagatcctgatcgagatctgcggccacaaggccatcggcaccgtgctcgtgggccccacccccgtgaacatcatcggccgcaacctgctgacccagatcggctgcaccctgaacttccccatctcccccatcgagaccgtgcccgtgaagctgaagcccggcatggacggccccaaggtgaagcagtggcccctgaccgaggagaagatcaaggccctggtggagatctgcaccgagatggagaaggagggcaagatctccaagatcggccccgagaacccctacaacacccccgtgttcgccatcaagaagaaggactccaccaagtggcgcaaactggtggacttccgcgagctgaacaagcgcacccaggacttctgggaggtgcagctgggcatcccccaccctgccggcctgaagaagaagaagtccgtgaccgtgctggacgtgggcgacgcctacttctccgtgcccctggacgagggcttccgcaagtacaccgccttcaccatcccctccatcaacaacgagacccccggcatccgctaccagtacaacgtgctgccccagggctggaagggctcccccgccatcttccagtcctccatgaccaagatcctggagcccttccgcgcccagaaccccgagatcgtgatctaccagtacatggacgacctgtacgtgggctccgacctggagatcggccagcaccgcatggagaaccgctggcaggtgatgatcgtgtggcaggtggaccgcatgcgcatccgcacctggaagtccctggtgaagcaccacctgaccgaggaggccgagctggagctggccgagaaccgcgagatcctgaaggaccccgtgcacggcgtgtactacgacccctccaaggacctgatcgccgagatccagtactggcaggccacctggatccccgagtgggagttcgtgaacaccccacccctggtgaagctgtggtaccagctggagaagaacgtgaccgagaacttcaacatgtggaagaacgacatggtggaccagatgcacgaggacatcatctccctgtgggaccagtccctgaagccctgcgtgaagctgaccccctgggtgcccgcccacaagggcatcggcggcaacgagcaggtggacaagctggtgtcccagggcatccgcaaggtgctgttcctggacggcatcgacaaggcccaggccaaggagatcgtggcctcctgcgacaagtgccagctgaagggcgaggccatgcacggccaggtggactgctcccccggcatctggcagctggactgcacccacctggagggcaaggtgatcctggtggccgtgcacgtggcctccggctacatcgaggccgaagtgattcccgccgagaccggccaggagaccgcctacttcctgctgaagctggccatgaacaaggagctgaagaagatcatcggccaggtgcgcgaccaggccgagcacctgaagaccgccgtgcagatggccgtgttcatccacaacttcaagcgcaagggcggaatcggcggctactccgccggcgagcgcatctggaagggccccgccaagctgctgtggaagggcgagggcgccgtggtgatccaggacaactccgacatcaaggtggtgccccgccgcaaggccaagatcatccgcgactacggcaagcagatggccggtgccgactgcgtgttcctgggcgctgccggctccaccatgggcgccgcctccatgaccctgaccgtgcaggcccgccagctgctgtccggcatcgtgcagcagcagaacaacctgctgcgcgccatcgaggcccagcagcacctgctgcagctgaccgtgtggggcatcaagcagtaatgataacccgggaagcttggaggcaag

SEQ ID NO: 32 - DNA, 975 bp, Mycobacterium bovis
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccctcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatgggcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaagcccgcgggcgggcacaacgccgtgttcaacttcccgcccaacggcacgcacagctgggagtactggggcgctcagctcaacgccatgaagggtgacctgcagagttcgttaggcgccggctgaaag

SEQ ID NO: 33 - DNA, 1059 bp, Plasmodium berghei
taccgccaccatgaagaaatgcaccatcctggtggtggccagcctgctgctggtcaactccctgctgcccggctacggccagaacaagagcatccaggcccagagaaacctgaacgagctgtgctacaacgagggcaacgacaacaagctgtaccacgtgctgaacagcaagaacggcaaaatctacatccggaacaccgtgaacagactgctggccgacgcccccgagggcaagaagaacgagaagaagaacaagatcgagcggaacaacaagctcaagcagccccctcccccccctaaccccaacgaccctcctcctcccaaccctaacgatccccccccacccaacccaaatgaccccccacccccaaatcctaacgacccaccccctcctaatcccaatgatcccccacctcctaacgcaaacgatcctccccctccaaatgccaacgaccccgctccacctaacgccaatgaccctgcccccccaaacgctaatgatccagccccacccaatgctaacgatccacctcccccgaacgccaacgatccgccaccgcctaatccgaatgacccagcacccccgaatgcaaatgacccacctccaccaaacccgaatgatcctgcacctccccagggcaacaacaacccccagccccagcccagaccccagcctcagccacagccacagccccagccacagcctcagccgcagccgcagccaaggccgcagcctcagccccagcctggcggcaacaacaacaacaagaacaacaacaatgacgacagctacatccccagcgccgagaagatcctggaatttgtgaagcagatccgggacagcatcaccgaggaatggtcccagtgcaacgtgacctgcggcagcggcatcagagtgcggaagagaaagggcagcaacaagaaggccgaggacctgaccctggaagatatcgacaccgaaatctgcaagatggacaagtgcagcagcatcttcaacatcgtgtccaacagcctgggcttcgtgatcctgctggtgctggtgttcttcaactgatgagc